SEQUENCE LISTING (32 sequences)

SEQ ID NO: 1 - DNA, 3509 bp, Artificial Sequence
  pM1 plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagagaattcacattaccctgttatccctagatgacattaccctgttatcccagatgacattaccctgttatccctagatgacattaccctgttatccctagatgacatttaccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagatgacattaccctgttatccctagatacattaccctgttatcccagatgacataccctgttatccctagatgacattaccctgttatcccagataaactcaatgatgatgatgatgatggctcttcaaagcaatgagtcgagactcagcggccgcggtgccagggcgtgcccttgggctccccgggcgcgactatatctagatatcggatcccgggcccgtcgactgcagaggcctgcatgcaagcttggggcccgccccaactggggtaacctttgagttctctcagttgggggtaatcagcatcatgatgtggtaccacatcatgatgctgattataagaatgcggccgccacactctagtggatctcgagttaataattcgctcttcaaagcaatgaaagcttggtgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaagcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgca

SEQ ID NO: 2 - DNA, 20 bp, Artificial Sequence
  Junction sequence
gggtaacctttgggctcccc

SEQ ID NO: 3 - DNA, 15 bp, Artificial Sequence
  RCA primer
gtaacctttgggctc

SEQ ID NO: 4 - DNA, 35 bp, Artificial Sequence
  attP attachment site
ggtgccagggcgtgcccttgggctccccgggcgcg

SEQ ID NO: 5 - DNA, 35 bp, Artificial Sequence; identical to SEQ ID NO: 4
  attB attachment site

SEQ ID NO: 6 - DNA, 1177 bp, Artificial Sequence
  ActEVEN pseudogene
ggtctcacattgcataatcttcatccgtgggaaccacggatgaagaaccgtcccaagcgttgcgccacatctgctggaaggtggacagtgagaggacacctacgaatcgcaacgggtatcctcatccgtgggaaccacggatgaggaaccgtcccaagcgttgcgcctgggtacatggtggtaccaccagacaggacacctacgaatcgcaacgggtatcctcatccgtgggaaccacggatgaggaaccgtcccaagcgttgcggagagcatagccctcgtagatgggcaaggacacctacgaatcgcaacgggtatcctcatccgtgggaaccacggatgaggaaccgtcccaagcgttgcggtcccagttggtaacaatgccatgttcaatgaggacacctacgaatcgcaacgggtatcctcatccgtgggaaccacggatgaggaaccgtcccaagcgttgcgcggactcatcgtactcctgcttgctgaggacacctacgaatcgcaacgggtatcctcatccgtgggaaccacggatgaggaaccgtcccaagcgttgcgttctctttgatgtcacgcacgatttcccaggacacctacgaatcgcaacgggtatcctcatccgtgggaaccacggatgaggaaccgtcccaagcgttgcgctcggtcaggatcttcatgaggtagtctgtaggacacctacgaatcgcaacgggtatcctcatccgtgggaaccacggatgaggaaccgtcccaagcgttgcgtttcacggttggccttagggttcaggggaggacacctacgaatcgcaacgggtatcctcatccgtgggaaccacggatgaggaaccgtcccaagcgttgcggtacttcagggtcaggatacctctcttgaggacacctacgaatcgcaacgggtatcctcatccgtgggaaccacggatgaggaaccgtcccaagcgttgcgctgctcgaagtctagagcaacatagcacaaggacacctacgaatcgcaacgggtatcctcatccgtgggaaccacggatgaggaaccgtcccaagcgttgcgcctcgtcacccacataggagtccttcaggacacctacgaatcgcaacgggtatcctcatccgtgggaaccacggatgaggagctcttcatgcattggagacc

SEQ ID NO: 7 - DNA, 1406 bp, Artificial Sequence
  CRC1 pseudogene
ggtctcacattgcataaaattcatccgtgggaaccacggatgaatttttctaatgtgccctatacaccccagccaagccaagatagcatctgaccttacccatcttacattattttgcttaacctctctccaacaaagaagttaaaactttatagaatgcattattcctgagcaatagactttttaataaaatacctacgtcttacattctttgttaggtcacctatgaccgtgtaaagtccaacatgcctgttacaagaaaaaaaaaaaatccagcacaacctagagttataatccaatctttatttaaaaatctaatctgccagtttacttgtacagctcgtccatgcccagggtgatgccagcggcggtcacaaattccagcagcaccatgtggtcccgcttctcgttggggtccttgctcagcttgctctgggtgctcaggtagtggttgtcgggcagcagcacggggccatctccgatgggggtgttctgctggtagtggtcggccagctgcacggagccgtcctcgatgttgtgccgggtcttgaagttcactttgatgccgtttttctgcttgtcggcggtgatgtacaccttgtggctgttgtagttgtactccagcttgtggcccaggatattgccgtcctctttgaagtcgatgcccttcagctcgatccggttcacgagggtgtcgccctcgaacttcacttcggccctggtcttgtagttgccgtcgtccttaaagaagatggtccgttcctgcacgtagccctcgggcatggcgctcttgaagaagtcgtgctgcttcatatggtcggggtatctggcgaagcactgcacgccgtaggtaaaggtggtcacgagggtaggccaaggcacgggcagcttgccggtggtgcagatgaacttcagggtcagcttgccatatgtggcgtcgccttcgccctcgccggacacgctgaacttgtggccgttcacgtcgccgtcgagttccaccagaatgggcaccacgccggtgaacagttcctcgcccttgctcacgcttcctccgcttccacctccgcttccaccgcgttttccaccaactcggggagctgaaactttcacaggcttcacaatcttttgcttaggtgctgcctttgtaggtgcctgtaaaaagataacacaatattactccacaaaacttttgttaccctattattacccatctctcacgtccagattgtttatgtagcttagatcactttcttagtaatttaactgcattcctaatgccaatcttttaatgtgactacccttaaatatttaaagcccatctttacaatgaaagacaaagactaagataccctcacagactcatccgtgggaaccacggatgagtcgctcttcatgcattggagacc

SEQ ID NO: 8 - DNA, 2059 bp, Artificial Sequence
  CRC2 pseudogene
ggtctcacattgcataatattcatccgtgggaaccacggatgaataaccaaaaagttaaggcaattaagtttttctaatgtgccctatacaccccagccaagccaagatagcatctgaccttacccatcttacattattttgcttaacctctctccaacaaagaagttaaaactttatagaatgcattattcctgagcaatagactttttaataaaatacctacgtcttacattctttgttaggtcacctatgaccgtgtaaagtccaacatgcctgttacaagaaaaaaaaaaaatccagcacaacctagagttataatccaatctttatttaaaaatctaatctgccagtttaggcgccaggctttctggtcatgcaccaggttctagggccctcaggcacttccacgtcggcggtcacggtgaagcccagtctctcgtagaagggcaggtttctgggggcgcttgtttccaggaaggcgggcacgccagccctttcagcagcttccacgccaggcagcaccacagcagatcccagtcccttgccctggtggtcaggtgacacgcccacggtggccagaaaccaggcaggctctttaggtctgtggggggccagcaggccttccatctgctgctgggcagccagtctagagccgctcagctcggccattctaggtccgatctcggcgaacacagcgccggcttccacagactcaggggttgtccacacagccacagcggcgccatcatcggccacccacactttgccgatgtccaggcccactctggtcagaaacagttcctgcagctcggtcactctctcgatgtgtctgtcggggtccacggtgtgtctggtggcagggtaatcggcgaaggcagcggccagtgtccgcacagctctaggcacatcgtccctggtggccagccgcactgtgggcttgtactcggtactaccaccgctccctccaccactgcctcccttgtacagctcgtccatgcccagggtgatgccagcggcggtcacaaattccagcagcaccatgtggtcccgcttctcgttggggtccttgctcagcttgctctgggtgctcaggtagtggttgtcgggcagcagcacggggccatctccgatgggggtgttctgctggtagtggtcggccagctgcacggagccgtcctcgatgttgtgccgggtcttgaagttcactttgatgccgtttttctgcttgtcggcggtgatgtacaccttgtggctgttgtagttgtactccagcttgtggcccaggatattgccgtcctctttgaagtcgatgcccttcagctcgatccggttcacgagggtgtcgccctcgaacttcacttcggccctggtcttgtagttgccgtcgtccttaaagaagatggtccgttcctgcacgtagccctcgggcatggcgctcttgaagaagtcgtgctgcttcatatggtcggggtatctggcgaagcactgcacgccgtaggtaaaggtggtcacgagggtaggccaaggcacgggcagcttgccggtggtgcagatgaacttcagggtcagcttgccatatgtggcgtcgccttcgccctcgccggacacgctgaacttgtggccgttcacgtcgccgtcgagttccaccagaatgggcaccacgccggtgaacagttcctcgcccttgctcacgcttcctccgcttccacctccgcttccaccgcgttttccaccaactcggggagctgaaactttcacaggcttcacaatcttttgcttaggtgctgcctttgtaggtgcctgtaaaaagataacacaatattactccacaaaacttttgttaccctattattacccatctctcacgtccagattgtttatgtagcttagatcactttcttagtaatttaactgcattcctaatgccaatcttttaatgtgactacccttaaatatttaaagcccatctttacaatgaaagacaaagactaagataccctcacagactggatcatccgtgggaaccacggatgatccgctcttcatgcattggagacc

SEQ ID NO: 9 - DNA, 378 bp, Artificial Sequence
  Single stranded oligonucleotide
ccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtg

SEQ ID NO: 10 - DNA, 420 bp, Artificial Sequence
  Single stranded oligonucleotide
attgaagcatgcggcgtgcataattctcttactgtcatgccatgcgtaagataccaccacacccgcattcgccattcaggcggccgccaccgcggtggagctccagctgctgtttcctgtgtagagttggtagctcttgatccggtcatatttgttccctttagatccgcctccatctacagggcgcgtccccgcgcttaatgcgcggcctaactacggctacactagaaggacttaccttcggaaaagaaattgttatccgctcacaaaagccagagtatttaagctccctcgtgcgctctcctgttccgggttattgtctcatcggcgaccgagttgctcttgcttatcagaccctgccgcttacaagtggtcgccagtctattaacagcactcaatacgggataatttttcaatatt

SEQ ID NO: 11 - DNA, 78 bp, Artificial Sequence
  Single stranded oligonucleotide
gaaccgtcccaagcgttgcgccacatctgctggaaggtggacagtgagaggacacctacgaatcgcaacgggtatcct

SEQ ID NO: 12 - DNA, 77 bp, Artificial Sequence
  Single stranded oligonucleotide
gaaccgtcccaagcgttgcgcctgggtacatggtggtaccaccagacaggacacctacgaatcgcaacgggtatcct

SEQ ID NO: 13 - DNA, 76 bp, Artificial Sequence
  Single stranded oligonucleotide
gaaccgtcccaagcgttgcggagagcatagccctcgtagatgggcaaggacacctacgaatcgcaacgggtatcct

SEQ ID NO: 14 - DNA, 81 bp, Artificial Sequence
  Single stranded oligonucleotide
gaaccgtcccaagcgttgcggtcccagttggtaacaatgccatgttcaatgaggacacctacgaatcgcaacgggtatcct

SEQ ID NO: 15 - DNA, 76 bp, Artificial Sequence
  Single stranded oligonucleotide
gaaccgtcccaagcgttgcgcggactcatcgtactcctgcttgctgaggacacctacgaatcgcaacgggtatcct

SEQ ID NO: 16 - DNA, 78 bp, Artificial Sequence
  Single stranded oligonucleotide
gaaccgtcccaagcgttgcgttctctttgatgtcacgcacgatttcccaggacacctacgaatcgcaacgggtatcct

SEQ ID NO: 17 - DNA, 80 bp, Artificial Sequence
  Single stranded oligonucleotide
gaaccgtcccaagcgttgcgctcggtcaggatcttcatgaggtagtctgtaggacacctacgaatcgcaacgggtatcct

SEQ ID NO: 18 - DNA, 78 bp, Artificial Sequence
  Single stranded oligonucleotide
gaaccgtcccaagcgttgcgtttcacggttggccttagggttcaggggaggacacctacgaatcgcaacgggtatcct

SEQ ID NO: 19 - DNA, 78 bp, Artificial Sequence
  Single stranded oligonucleotide
gaaccgtcccaagcgttgcggtacttcagggtcaggatacctctcttgaggacacctacgaatcgcaacgggtatcct

SEQ ID NO: 20 - DNA, 79 bp, Artificial Sequence
  Single stranded oligonucleotide
gaaccgtcccaagcgttgcgctgctcgaagtctagagcaacatagcacaaggacacctacgaatcgcaacgggtatcct

SEQ ID NO: 21 - DNA, 76 bp, Artificial Sequence
  Single stranded oligonucleotide
gaaccgtcccaagcgttgcgcctcgtcacccacataggagtccttcaggacacctacgaatcgcaacgggtatcct

SEQ ID NO: 22 - DNA, 461 bp, Artificial Sequence
  Pseudogene
ggtctcacattgcataattaacatccgcggaacgcggatgttccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgcatccgcggaacgcggatgcagctcttctgcattggagacc

SEQ ID NO: 23 - DNA, 510 bp, Artificial Sequence
  Pseudogene
ggtctcacattgcataaatttcatccgtgggaaccacggatgaaattgaagcatgcggcgtgcataattctcttactgtcatgccatgcgtaagataccaccacacccgcattcgccattcaggcggccgccaccgcggtggagctccagctgctgtttcctgtgtagagttggtagctcttgatccggtcatatttgttccctttagatccgcctccatctacagggcgcgtccccgcgcttaatgcgcggcctaactacggctacactagaaggacttaccttcggaaaagaaattgttatccgctcacaaaagccagagtatttaagctccctcgtgcgctctcctgttccgggttattgtctcatcggcgaccgagttgctcttgcttatcagaccctgccgcttacaagtggtcgccagtctattaacagcactcaatacgggataatttttcaatattcatccgtgggaaccacggatgaatagctcttcatgcattggagacc

SEQ ID NO: 24 - DNA, 1177 bp, Artificial Sequence; identical to SEQ ID NO: 6
  Pseudogene

SEQ ID NO: 25 - DNA, 7 bp, Artificial Sequence
  Nt.BspQI cleavage domain
gctcttc

SEQ ID NO: 26 - DNA, 6 bp, Artificial Sequence
  Nb.BsrDI cleavage domain
gcaatg

SEQ ID NO: 27 - DNA, 5455 bp, Artificial sequence
  pM2
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggggggttctcatcatcatcatcatcatggtatggctagcccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttcgatattgtcaataaatcactccagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaataaacaggaataccaattattaaaagataacttagtcagatcgtacaataaagccttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataacgcaaaaaaccccgcttcggcggggttttttcgctaactataacggtcctaaggtagcgagaagttcctatactttttagagaataggaacttcctcgaggtaccagaattatcggatcccgggcccgtcgagaagttcctatactttttagagaataggaacttcttaattaaggcgcgcccaattgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 28 - DNA, 5041 bp, Artificial sequence
  pM3
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggggggttctcatcatcatcatcatcatggtatggctagcgcgacccgcgaacagcagccggaaggccgccgcctgaaagtggcgcgcatttatctgcgcgcgagcaccgatgaacagaacctggaacgccaggaaagcctggtggcggcgacccgcgcggcgggctattatgtggcgggcatttatcgcgaaaaagcgagcggcgcgcgcgcggatcgcccggaactgctgcgcatgattgcggatctgcagccgggcgaagtggtggtggcggaaaaaattgatcgcattagccgcctgccgctggcggaagcggaacgcctggtggcgagcattcgcgcgaaaggcgcgaaactggcggtgccgggcgtggtggatctgagcgaactggcggcggaagcgaacggcgtggcgaaaattgtgctggaaagcgtgcaggatatgctgctgaaactggcgctgcagatggcgcgcgatgattatgaagatcgccgcgaacgccagcgccagggcgtgcagctggcgaaagcggcgggccgctataccggccgcaaacgcgatgcgggcatgcatgatcgcattattaccctgcgcagcggcggcagcagcattgcgaaaaccgcgaaactggtgggctgcagcccgagccaggtgaaacgcgtgtgggcggcgtggaacgcgcagcagcagaaataacgcaaaaaaccccgcttcggcggggttttttcgctaactataacggtcctaaggtagcgaggtcaaattgggtatacccatttgggcctagtctagccggcatggcgcattacagcaatacgcaatttaaatgcgcctagcgcattttcccgaccttaatgcgcctcgcgctgtagcctcacgcccacatatgctcgaggtaccagaattatcggatcccgggcccgtcgaggtcaaattgggtatacccatttgggcctagtctagccggcatggcgcattacagcaatacgcaatttaaatgcgcctagcgcattttcccgaccttaatgcgcctcgcgctgtagcctcacgcccacatatgttaattaaggcgcgcccaattgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 29 - DNA, 133 bp, Artificial sequence
  msr_l (ParA resolvase attachment site)
ggtcaaattgggtatacccatttgggcctagtctagccggcatggcgcattacagcaatacgcaatttaaatgcgcctagcgcattttcccgaccttaatgcgcctcgcgctgtagcctcacgcccacatatg

SEQ ID NO: 30 - DNA, 133 bp, Artificial sequence; identical to SEQ ID NO: 29
  msr_r (ParA resolvase attachment site)

SEQ ID NO: 31 - DNA, 34 bp, Artificial sequence
  FLPr (FLP recombinase attachment site)
gaagttcctatactttttagagaataggaacttc

SEQ ID NO: 32 - DNA, 34 bp, Artificial sequence; identical to SEQ ID NO: 31
  FLPl (FLP recombinase attachment site)

(19) United States
(12) Patent Application Publication
(10) Pub. No.: US 2023/0079822 A1
(43) Pub. Date: Mar. 16, 2023

(54) METHOD AND PRODUCTS FOR PRODUCING SINGLE STRANDED DNA POLYNUCLEOTIDES

(71) Applicant: MOLIGO TECHNOLOGIES AB, Solna (SE)

(72) Inventors: Cosimo DUCANI, Solna (SE); Giulio BERNARDINELLI, Solna (SE); Björn HÖGBERG, Solna (SE)

(21) Appl. No.: 17/801,794

(22) PCT Filed: Feb. 24, 2021
(86) PCT No.: PCT/EP2021/054557

§ 371 Date: Aug. 24, 2022

(30) Foreign Application Priority Data
Feb. 24, 2020 (GB) 2002547.4

(51) Int. Cl. C12N 15/64; C12N 15/10; C12N 15/70; C12N 15/79

(52) U.S. Cl. CPC C12N 15/64; C12N 15/10; C12N 15/70; C12N 15/79

(57) ABSTRACT

The present invention provides a method for producing single stranded DNA polynucleotides. In particular, the invention provides a method that utilises a DNA minicircle obtained from a parental minicircle plasmid as a template in an enzyme-mediated rolling circle amplification (RCA) reaction to generate a product that can be cleaved to provide the plurality of single stranded DNA polynucleotides.

The present invention relates to a method for producing single stranded DNA polynucleotides. In particular, the invention provides a method that utilises a DNA minicircle obtained from a parental minicircle plasmid as a template in an enzyme-mediated rolling circle amplification (RCA) reaction to generate a plurality of single stranded DNA polynucleotides (e.g. a library comprising a plurality of different single stranded DNA polynucleotides). The invention also provides the use of a parental minicircle plasmid in the production of a plurality of single stranded DNA polynucleotides. A parental minicircle plasmid, a kit for use in the method (e.g. comprising the parental minicircle plasmid), and single stranded DNA polynucleotides obtained by the method are also provided. Furthermore, the invention provides a library comprising a mixture of single stranded DNA polynucleotides (e.g. functionalised single stranded DNA polynucleotides) obtained by the method.

Single stranded DNA polynucleotides are useful in a wide range of applications due to their ability to hybridize, via Watson-Crick base pairing, to complementary sequences, e.g. intermolecularly. In addition, single stranded polynucleotides can hybridize to themselves, (i.e. intramolecular complementarity) so as to form complex topological geometries and molecular assemblies, including secondary structures known as aptamers. These aptamers can bind with high affinity to biological targets via non-covalent interactions to effect a range of different functions.

At present, single stranded DNA polynucleotides may be produced using solid-phase synthesis, wherein nucleotides are added in a stepwise manner to a growing chain bound to a solid support. However, this method can be severely limited in terms of the length and accuracy of the polynucleotides produced. The error rate in the production of polynucleotides by solid-phase synthesis is significantly higher than the error rate of polymerase enzymes observed in nature, and increases dramatically with the length of the polynucleotide, such that purities of only 70% are common in commercial polynucleotides of around 50 nucleotides in length. This error rate makes solid-phase synthesis methods unsuitable for the production of longer polynucleotides.

As an alternative to such solid-phase synthesis methods, the present inventors have previously described a method for the enzymatic production of ‘monoclonal stoichiometric’ (MOSIC) single stranded DNA oligonucleotides from sequence-verified templates (Ducani et al., 2013, Nature Methods, 647-652). In a representative example, this MOSIC method involves the design and preparation of a linear sequence comprising one or more oligonucleotide sequences to be produced, each bordered by hairpin sequences containing a restriction enzyme site. The linear sequence is then circularised into a double stranded rolling circle amplification (RCA) template, which is nicked and amplified by RCA to produce a partially single stranded linear concatemer comprising a plurality of copies of the single stranded oligonucleotides to be produced. Finally, the RCA product is treated with a restriction enzyme which recognises the restriction site in the aforementioned hairpin regions, and cleaves the concatemer to release a plurality of single stranded oligonucleotides. If the linear sequence contains more than one oligonucleotide sequence, the cleavage reaction results in a mixture of the desired oligonucleotides. The ratio of oligonucleotides in the mixture is determined by the number of each oligonucleotide sequence in the original linear sequence.

The step in the MOSIC reaction of circularising the linear sequence to produce a circular RCA template is typically done by a conventional ligation reaction using a ligase enzyme. However, in work leading to the present invention, the inventors determined that conventional ligation reactions become less efficient at producing circularised RCA templates as the linear molecule to be circularised is increased in length. For example, intramolecular circularization of linear molecules with a length of about 1 kilobase (kb) or longer using conventional ligation reactions is very inefficient. With these longer linear templates, the ligation reaction primarily results in intermolecular, rather than intramolecular, ligations and thus predominantly produces linear concatemers, rather than circularised DNA molecules. As a result of this, where the linear sequence used to produce the RCA template is longer (around 1 kb or more in length), e.g. where it is desired to produce longer polynucleotides and/or a large plurality of oligonucleotides with different sequences, the method must include further steps following the ligation reaction of isolating the circular DNA molecules from the linear concatemers, and concentrating the circular DNA molecules such that they can be used as RCA templates. These additional steps make the process more time-consuming and therefore more expensive. In some instances, it may not be feasible to produce enough circular DNA molecules to template an RCA reaction.

In view of these issues, there is a desire for alternative methods of producing single stranded polynucleotides, particularly methods which are effective generating longer polynucleotides (e.g. of about 1 kb or longer) and/or a large plurality of oligonucleotides with different sequences.

In the context of gene therapy, it has been demonstrated that superior transfection yield and a prolonged period of expression of a transgene in a host cell can be achieved by the removal of bacterial gene cassettes (such as the origin of replication, antibiotic resistance genes, etc., which are predominantly required for the propagation of the plasmid itself) from a recombinant plasmid comprising the transgene of interest (Mayrhofer et al., 2009, Gene Therapy of Cancer, 87-104).

Accordingly, so-called minicircle systems have been designed to take advantage of bacterial machinery to both propagate a plasmid and to excise the aforementioned undesired DNA fragments from the plasmid, so as to produce a smaller plasmid, known as a DNA minicircle, comprising the transgene expression cassette (Kay et al., 2010, Nature Biotechnology, 1287-1289 which is incorporated herein by reference). Thus, the initial plasmid may be viewed as a “parental minicircle plasmid”, giving rise to a DNA minicircle.

These minicircle systems typically involve the use of a recombinase enzyme (sometimes called an “integrase”) which mediates a recombination reaction between two recombinase attachment sites present in the parental minicircle plasmid. The parental minicircle plasmid is designed such that it comprises a transgene expression cassette, flanked (bordered) by the recombinase attachment sites. A recombination reaction between the two recombinase attachment sites generates a DNA minicircle comprising the intervening transgene. The DNA minicircle is thus effectively excised from the larger parental minicircle plasmid, leaving behind the now unnecessary bacterial gene cassettes. The DNA minicircle can be used to transfect cells and therefore improve the efficiency of the subsequent gene therapy.

The inventors have surprisingly determined that minicircle systems can be adapted to produce circularised RCA templates suitable for the efficient production of single stranded DNA polynucleotides, particularly longer polynucleotides (e.g. of about 1 kb or longer). In this regard, the inventors have developed parental minicircle plasmids, known as pM1, pM2 and pM3 (SEQ ID NOs: 1, 27 and 28 respectively), which find particular utility in the generation of RCA templates for use in the MOSIC method for producing single stranded DNA polynucleotides.

The generation of RCA templates for the production of single stranded DNA polynucleotides using a minicircle system has a number of advantages over the original MOSIC method. The MOSIC method typically involves the production of a linear sequence comprising the oligonucleotide sequences to be produced, which is inserted into a plasmid so that its sequence can be checked (e.g. by sequencing) and so that it can be amplified via propagation in bacterial cells. The plasmid is then purified from the bacteria, and the sequence comprising the oligonucleotide sequences to be produced is excised from the plasmid, purified and then re-circularised (this re-circularisation process typically includes further steps of purification, as discussed above) to form the RCA template.

By adapting the minicircle system for use in the present method, the inventors have been able to dramatically reduce the number of steps in this process. The DNA minicircle which acts as the RCA template in the MOSIC method can be isolated directly from a bacterial culture comprising the parental minicircle plasmid, without the need for excision, purification and re-circularisation. Notably, the DNA minicircle can also be produced from a parental minicircle plasmid in vitro. In some embodiments, the parental minicircle plasmid may be arranged or configured to enable the degradation of any intact parental minicircle plasmid (i.e. plasmid that has not recombined) and the backbone plasmid (i.e. the region of the parental minicircle plasmid outside of the recombinase attachment sites), following the excision of the DNA minicircle. This ensures that only the excised DNA minicircle is able to function as a template for RCA. In some embodiments, the RCA reaction may utilise a primer that is complementary only to a region of the excised DNA minicircle. Thus, in some embodiments, it is not necessary to separate the DNA minicircle following its excision from the parental minicircle plasmid. Accordingly, the method reduces the need for additional purification steps and allows the RCA templates to be produced at a higher yield.

Thus, the inventors have determined that the minicircle approach enables the efficient production of larger circularised RCA templates, e.g. templates that are about 1 kb or more in length. This is particularly advantageous for the production of longer single stranded DNA polynucleotides (e.g. that are about 1 kb or more in length) and/or for the production of a large plurality of oligonucleotides with different sequences in a single reaction.

Accordingly, at its broadest, the invention can be seen to provide the use of a DNA minicircle obtained from a parental minicircle plasmid in the production of a plurality of single stranded DNA polynucleotides, wherein the DNA minicircle comprises the polynucleotide sequence to be produced, bordered by cleavage domains. The DNA minicircle is used in an RCA reaction to generate an RCA product that can be cleaved to produce the plurality of single stranded DNA polynucleotides.

More particularly, the invention can be seen to provide the use of a plurality of DNA minicircles obtained from a plurality of parental minicircle plasmids in the production of a plurality of single stranded DNA polynucleotides, wherein each DNA minicircle comprises the polynucleotide sequence to be produced, bordered by cleavage domains. The DNA minicircles are used in an RCA reaction to generate RCA products that can be cleaved to produce the plurality of single stranded DNA polynucleotides.

Alternatively viewed, the invention can be seen to provide the use of a parental minicircle plasmid in the production of a plurality of single stranded DNA polynucleotides, wherein a DNA minicircle obtained from the parental minicircle plasmid comprises the polynucleotide sequence to be produced, bordered by cleavage domains.

More particularly, the invention provides the use of a plurality of parental minicircle plasmids in the production of a plurality of single stranded DNA polynucleotides, wherein a plurality of DNA minicircles obtained from the parental minicircle plasmids each comprise the polynucleotide sequence to be produced, bordered by cleavage domains.

Alternatively viewed the present invention provides a method for producing a plurality of single stranded DNA polynucleotides, said method comprising:

a) providing a DNA minicircle obtained from a parental minicircle plasmid, wherein the DNA minicircle comprises the polynucleotide sequence to be produced bordered by cleavage domains;

b) performing a rolling circle amplification (RCA) reaction with the DNA minicircle of (a) as a template to produce an RCA product comprising a plurality of copies of the polynucleotide sequence to be produced, bordered by cleavage domains; and

c) cleaving the RCA product at the cleavage domains to release the plurality of single stranded DNA polynucleotides.

Thus, more particularly the present invention provides a method for producing a plurality of single stranded DNA polynucleotides, said method comprising:

a) providing a plurality of DNA minicircles obtained from a plurality of parental minicircle plasmids, wherein each DNA minicircle comprises the polynucleotide sequence to be produced bordered by cleavage domains;

b) performing a rolling circle amplification (RCA) reaction with the DNA minicircles of (a) as templates to produce a plurality of RCA products each comprising a plurality of copies of the polynucleotide sequence to be produced, bordered by cleavage domains; and

c) cleaving the RCA products at the cleavage domains to release the plurality of single stranded DNA polynucleotides.

The term “plasmid” refers to an extrachromosomal circular DNA molecule capable of replicating autonomously in a host cell, e.g. a prokaryotic or bacterial cell.

The term “parental minicircle plasmid” refers to a plasmid that can recombine in the presence of a site-specific recombinase enzyme (and under suitable conditions) to generate two circular DNA molecules, both of which are smaller than the original plasmid. Typically, a parental minicircle plasmid contains a first domain comprising a polynucleotide of interest bordered by recombinase attachment sites upon which the recombinase enzyme acts, and a second domain comprising elements required for replication, propagation and retention of the plasmid within the host cell, e.g. origin of replication, selective marker (e.g. antibiotic resistance gene) etc. As discussed below, a parental minicircle plasmid may contain additional elements, particularly in the second domain. Thus, recombination of the parental minicircle plasmid results in a first circular DNA molecule (circle) comprising the polynucleotide of interest, a so-called “minicircle” and a second circular DNA molecule (circle) comprising elements required for replication, propagation and retention within the host cell, i.e. the plasmid backbone. Thus, a parental minicircle plasmid may also be viewed as a “minicircle producer plasmid” or a “minicircle plasmid” and these terms are used interchangeably herein.

In the present invention, the polynucleotide of interest in the first domain of the parental minicircle plasmid comprises the polynucleotide to be produced bordered by cleavage domains. This sequence, comprising one or more desired polynucleotide sequences bordered by cleavage domains, is referred to as a “pseudogene”.

Thus, the DNA minicircle provided in step (a) of the present method is produced from a parental minicircle plasmid through a recombination reaction. This recombination reaction is mediated by a recombinase enzyme, which recognises recombinase attachment sites in the parental minicircle plasmid, between which is a sequence comprising the polynucleotide to be produced, bordered by cleavage domains.

Accordingly, in the method of the present invention the step of providing a DNA minicircle may comprise several stages. For instance, in some embodiments, the invention may comprise a step of obtaining the DNA minicircle from a parental minicircle plasmid.

The step of obtaining the DNA minicircle from the parental minicircle plasmid may be achieved by any suitable means. As noted above, a DNA minicircle is produced by contacting the parental minicircle plasmid with a recombinase enzyme capable of recombining the parental minicircle plasmid via the attachment sites under conditions suitable for the recombination reaction. Thus, the recombination reaction may be performed in vitro. However, in preferred embodiments, the DNA minicircle is produced in vivo, e.g. by propagating the parental minicircle plasmid in a host cell (e.g. a bacterial cell) capable of expressing a site-specific recombinase enzyme that acts on the recombinase attachment sites in the parental minicircle plasmid. Thus, in some embodiments, step (a) may comprise providing a host cell comprising the parental minicircle plasmid, wherein the host cell is capable of expressing a site-specific recombinase enzyme that acts on recombinase attachment sites in the parental minicircle plasmid. More particularly, step (a) may comprise providing a plurality of host cells each comprising a plurality of copies of the parental minicircle plasmid, wherein the host cells are capable of expressing a site-specific recombinase enzyme that acts on recombinase attachment sites in the parental minicircle plasmids.

In some embodiments, the method may contain a step of amplifying the parental minicircle plasmid, e.g. propagating a host cell (e.g. a bacterial cell) comprising the parental minicircle plasmid. In some embodiments, the method may contain a step of inducing expression of a recombinase enzyme in the host cell (e.g. a bacterial cell) which functions to recombine the parental minicircle plasmid to form the DNA minicircle. Alternatively viewed, expression of the site-specific recombinase in the host cell promotes the formation of the DNA minicircle in the cell. The skilled person will be capable of selecting suitable conditions under which the bacteria may be propagated depending on the particular bacterial host that is used, as such conditions are well-known in the art.

The selection of a suitable host cell (e.g. bacterial cell) will depend on the structure of the parental minicircle plasmid. As noted above, the second domain of the parental minicircle plasmid may comprise a number of additional elements with various functionalities.

For example, in some embodiments, the parental minicircle plasmid may comprise a sequence encoding the recombinase enzyme that is to be used for the recombination reaction to generate the DNA minicircle (e.g. pM2 and pM3). Alternatively, in some embodiments, the parental minicircle plasmid may not comprise a sequence encoding a recombinase enzyme. Thus, in some embodiments, the parental minicircle plasmid is propagated in a host cell (e.g. a bacterial cell) which comprises, in its genome or on a separate plasmid, a sequence encoding the recombinase enzyme that recognises the recombinase attachment sites present in the parental minicircle plasmid.

In some embodiments, sequence encoding the recombinase enzyme is operably linked to an inducible promoter. The use of an inducible promoter ensures that the recombinase is not expressed (or is only minimally expressed) until enough cells comprising the parental minicircle plasmid have been grown to yield a sufficient amount of DNA minicircles to be used in the method of the invention. Thus, the step of inducing expression of a recombinase enzyme in the host cell(s) may comprise contacting the cell(s) with a substance that directly or indirectly promotes (e.g. increases) the expression of the recombinase enzyme.

In some embodiments, the step of obtaining the DNA minicircle from the parental minicircle plasmid comprises contacting the parental minicircle plasmid with a recombinase enzyme in vitro (e.g. in a solution in a reaction vessel), wherein the recombinase enzyme is capable of recombining the parental minicircle plasmid via the attachment sites. The step of contacting the parental minicircle plasmid with a recombinase enzyme is performed under conditions suitable for the recombination reaction.

When the step of obtaining the DNA minicircle from the parental minicircle plasmid is performed in vitro it may be preferred that the parental minicircle plasmid does not encode a recombinase enzyme. Thus, in some embodiments the parental minicircle plasmid does not encode a recombinase enzyme.

The skilled person readily could determine what in vitro conditions (e.g. buffer, temperature, reactant concentrations) are suitable for the recombination reaction and these conditions will differ depending on the recombinase enzyme used in the reaction. In some embodiments, suitable conditions include conditions that result in at least about a 30% yield of DNA minicircles, e.g. at least about a 40%, 50%, 60% or 70% yield of DNA minicircles, from the parental minicircle plasmid. Alternatively viewed, contacting the parental minicircle plasmid with a recombinase enzyme in vitro under suitable conditions results in the production of DNA minicircles from at least about 30%, e.g. at least about a 40%, 50%, 60% or 70%, of the parental minicircle plasmids in the in vitro reaction.

The term “recombinase” or “recombinase enzyme” refers to an enzyme that catalyzes a site-specific DNA exchange reaction between target site sequences (often termed “attachment sites”) that are specific to each recombinase. Any suitable recombinase enzyme that can participate in a recombination reaction using attachment sites on a plasmid to yield a DNA minicircle may be used in the methods of the present invention. In some embodiments, the recombinase is a serine integrase, such as PhiC31 integrase or ParA resolvase. In some embodiments, the recombinase is a tyrosine integrase, such as Cre recombinase, bacteriophage λ integrase or FLP recombinase. Suitable recombinase enzymes therefore include PhiC31 integrase, ParA resolvase, Cre recombinase, bacteriophage λ integrase, Hin recombinase, Tre recombinase and FLP recombinase.

As used herein, the term “recombinase” includes not only naturally occurring enzymes but also all such modified derivatives, including also derivatives of naturally occurring recombinase enzymes.

Particularly preferred recombinase enzymes for use in the invention include PhiC31 integrase, ParA resolvase, FLP recombinase and derivatives, e.g. sequence-modified derivatives, or mutants thereof.

Sequence-modified derivatives or mutants of recombinase enzymes include mutants that retain at least some of the functional activity of the wild-type sequence. Mutations may affect the activity profile of the enzymes, e.g. enhance or reduce the rate of recombination, under different reaction conditions, e.g. temperature, substrate concentration, pH etc. Mutations or sequence-modifications may also affect the thermostability of the enzyme.

In some embodiments, the recombinase enzyme may be PhiC31 integrase (e.g. UniProtKB accession No. Q9T221). This enzyme is a site-specific recombinase from the phiC31 bacteriophage, which recognises the recombinase attachment sites attB and attP (SEQ ID NOs: 4 and 5). Accordingly, in some embodiments, the parental minicircle plasmid comprises a sequence encoding PhiC31 integrase. In some embodiments, the host cell genome comprises a sequence encoding PhiC31 integrase or the host cell comprises a plasmid comprising a sequence encoding PhiC31 integrase. In some embodiments, the sequence encoding PhiC31 integrase is operably linked to an inducible promoter.

In some embodiments, the recombinase enzyme is ParA resolvase (e.g. UniProtKB accession No. P22996). This enzyme is a site-specific recombinase encoded on the IncP-alpha RP4 plasmid of E. coli, which recognises the recombinase attachment sites mrs_l and mrs_r (SEQ ID NOs: 29 and 30). Accordingly, in some embodiments, the parental minicircle plasmid comprises a sequence encoding ParA resolvase (e.g. pM3, SEQ ID NO: 28). In some embodiments, the host cell genome comprises a sequence encoding ParA resolvase or the host cell comprises a plasmid comprising a sequence encoding ParA resolvase. In some embodiments, the sequence encoding ParA resolvase is operably linked to an inducible promoter.

In some embodiments, the recombinase enzyme is FLP recombinase (e.g. UniProtKB accession No. P03870). This enzyme is a site-specific recombinase encoded by S. cerevisiae, which recognises the recombinase attachment sites FLPr and FLPl (SEQ ID NOs: 31 and 32). Accordingly, in some embodiments, the parental minicircle plasmid comprises a sequence encoding FLP recombinase (e.g. pM2, SEQ ID NO: 27). In some embodiments, the host cell genome comprises a sequence encoding FLP recombinase or the host cell comprises a plasmid comprising a sequence encoding FLP recombinase. In some embodiments, the sequence encoding FLP recombinase is operably linked to an inducible promoter.

As noted above, the parental minicircle plasmid may be propagated in any suitable host cell. The host cell may be a prokaryotic (e.g. bacterial) or eukaryotic (e.g. yeast) cell capable of supporting the replication of the plasmid, preferably at high copy numbers. In preferred embodiments, the cell is a prokaryotic cell, e.g. a bacterial cell, such as E. coli.

In some embodiments, a suitable host cell (e.g. bacterial cell) may have characteristics that facilitate the production of the DNA minicircle from the parental minicircle plasmid. For example, in some embodiments, the host cell may be capable of expressing a suitable recombinase, i.e. a recombinase capable of recognising the recombinase attachment sites in the parental minicircle plasmid. In some embodiments, the nucleic acid encoding the recombinase is operably linked to an inducible promoter, as described above. In preferred embodiments, the gene encoding the recombinase is integrated into the host cell genome. However, the gene encoding the recombinase may be provided on a plasmid within the host cell.

In some embodiments, the host cell may be capable of expressing an endonuclease (e.g. a homing endonuclease and/or nickase) that finds utility in the method, e.g. an endonuclease capable of recognising and cleaving the one or more cleavage domains in the parental minicircle plasmid. In some embodiments, the nucleic acid encoding the endonuclease is operably linked to an inducible promoter, as described above, e.g. the same inducible promoter system used to control the expression of the recombinase. In preferred embodiments, the gene encoding the endonuclease is integrated into the host cell genome.

The host cell may comprise other characteristics that facilitate the use of the parental minicircle plasmid system in the method of the invention. For instance, the host cell may be capable of expressing a polymerase enzyme that finds utility in the method, i.e. a DNA polymerase, as described in more detail below. The expression of the DNA polymerase may be under the control of an inducible promoter system. The inducible promoter system may be the same or different to the system used to control the expression of other genes in the host cell, e.g. recombinase and/or endonuclease.

It will be evident that a host cell capable of expressing enzymes that find utility in the method of the invention must be capable of expressing those enzymes in sufficient quantities to achieve the desired function. This may be achieved by any suitable means. For instance, the encoding sequences may be operably linked to strong promoters and/or the host cell may contain multiple copies of the encoding sequences, e.g. 2, 3, 4, 5 or more copies.

As noted above, in some embodiments, the expression of enzymes in the host cell, e.g. encoded by parental minicircle plasmid, encoded by a separate plasmid and/or in the host cell genome, may be controlled by an inducible expression system such that the expression of the enzymes can be readily controlled. Any suitable inducible expression system known in the art may be used and the selection of a suitable expression system is within the purview of the skilled person. It will be understood that the inducible expression system must be compatible with the host cell and parental minicircle plasmid. For example, in some embodiments, the inducible expression system may be an arabinose induction system (e.g. the L-arabinose-inducible araCBAD system). Accordingly, the host cell comprises an arabinose transporter, such that the inducer can be detected and expression can be triggered. In some embodiments, the host cell is an E. coli strain, such as an E. coli strain comprising the L-arabinose-inducible araCBAD system. In a preferred embodiment, the E. coli strain is ZYCY10P3S3T (see Kay et al., 2010, Nature Biotechnology 28(12), pp. 1287-1289 which is incorporated herein by reference). In some embodiments, the E. coli strain is DH10B, Top10 or LMG194.

It will be evident that once a parental minicircle plasmid containing the pseudogene has been produced, a stock of plasmid may be produced such that the plasmid may be used repeatedly to produce the DNA minicircles for use in the claimed method. In other words, it is not necessary to generate the parental minicircle plasmid containing the pseudogene de novo each time it is required to produce a plurality of single stranded polynucleotide(s) encoded by the pseudogene.

Nevertheless, in some embodiments, the step of providing a DNA minicircle may comprise steps of preparing a parental minicircle plasmid. Thus, in some embodiments, the method may therefore comprise a step of designing a pseudogene sequence, e.g. in silico. Thus, the method may utilise a computer-implemented method of designing the pseudogene. Such methods are described in the art, e.g. Ducani et al., 2013, supra. The term “pseudogene” as used herein refers to a nucleotide sequence comprising one or more desired polynucleotide sequences, bordered by cleavage domains. Alternatively, this sequence may be referred to herein as the “nucleic acid construct”.

The pseudogene sequence that is designed in silico can be produced (e.g. synthesised) using commercially available gene synthesis methods, or any other suitable means known in the art, e.g. assembly PCR. Thus, the present method may comprise a step of producing the pseudogene.

The pseudogene is then introduced into a parental minicircle plasmid, such that it can be amplified and subsequently converted into a DNA minicircle. Thus, the method may comprise a step of inserting the pseudogene into a parental minicircle plasmid. The insertion of the pseudogene into the parental minicircle plasmid may be done using any suitable means known in the art. For instance, the pseudogene may be ligated into a parental minicircle plasmid using a ligase enzyme, as described below. It will be understood in this regard that at least one of the 5′ ends of the pseudogene is phosphorylated to enable ligation to take place. In embodiments where the step of producing the pseudogene results in a DNA molecule in which the 5′ ends are not phosphorylated, the method may comprise a further step of phosphorylating the 5′ end(s) of the pseudogene, e.g. using a kinase enzyme, such as T4 polynucleotide kinase. It will be understood that in order for the pseudogene sequence to be retained in the DNA minicircle following the recombination reaction, it must be introduced into the parental minicircle plasmid such that it is located between the recombinase attachment sites. Accordingly, the pseudogene is introduced into the parental minicircle plasmid such that it is bordered by the attachment sites and the recombination reaction results in a DNA minicircle comprising the pseudogene. In this context, “bordered” means that the recombinase attachment sites are directly or indirectly adjacent to the pseudogene sequence.

The parental minicircle plasmid comprising the pseudogene sequence is then transformed into a suitable host cell (e.g. bacterial cell) as described above. This allows the sequence of the pseudogene to be checked (e.g. by sequencing) and for any errors in the sequence to be corrected, e.g. via iterations of sequencing and mutagenesis using any suitable methods. In addition, the process of growing bacteria comprising the parental minicircle plasmid is a useful amplification step, which facilitates the generation of a significant number of copies of the parental minicircle plasmid, and therefore ultimately of the DNA minicircle to act as a template for the RCA reaction.

In addition to amplification, the process of transfecting the parental minicircle plasmid comprising the pseudogene into bacteria also allows a bacterial glycerol stock to be produced. Bacteria comprising the desired pseudogene plasmid can be prepared in glycerol, frozen and stored stably for long periods of time.

Accordingly, in some embodiments step (a) of the present method comprises:

(i) cloning into a parental minicircle plasmid a linear DNA molecule comprising the polynucleotide sequence bordered by cleavage domains;

(ii) transfecting the parental minicircle plasmid obtained in step (i) into a host cell (e.g. bacterial cell);

(iii) amplifying said parental minicircle plasmid (e.g. propagating the host cell comprising the parental minicircle plasmid);

(iv) performing a recombination reaction to produce a DNA minicircle comprising the polynucleotide sequence bordered by cleavage domains (e.g. inducing expression of a recombinase enzyme in the host cell or contacting isolated parental minicircle plasmid with a recombinase enzyme in vitro); and, optionally,

(v) isolating the DNA minicircle obtained in step (iv).

As noted above, where a parental minicircle plasmid containing a pseudogene has been produced previously, step (i) is not required to obtain the DNA minicircle. Similarly, if the parental minicircle plasmid has been transfected into a host cell previously, e.g. to produce a glycerol stock, step (ii) is not required obtain the DNA minicircle, e.g. the host cell may be propagated directly from the glycerol stock to amplify the parental minicircle plasmid.

As step (iv) may be performed in vitro, the method may further include a step of isolating parental minicircle plasmid, e.g. from the host cell used to amplify the plasmid. Any means of isolating a plasmid from a host cell may be used in the isolation step and suitable means are well-known in the art.

The step of isolating the DNA minicircle may be achieved using any suitable means, which will depend on the level of purity required to perform subsequent steps of the methods of the invention, i.e. the RCA reaction, and on how the DNA minicircle was produced. For instance, in embodiments where the DNA minicircle is produced in a host cell, isolating the DNA minicircle may comprise a step of lysing the host cell to release the DNA minicircle. In some embodiments, the host cell lysate may be used directly in the RCA reaction. In some embodiments, the host cell lysate may be subjected to purification steps to obtain an enriched or purified preparation comprising the DNA minicircle.

In some embodiments, it may be necessary or advantageous to separate the DNA minicircle from the other products of the recombination reaction, i.e. the plasmid backbone, and any unreacted (intact) parental minicircle plasmid. In some embodiments, it may be necessary or advantageous to separate the DNA minicircle from components (e.g. cleavage enzymes, recombinase enzymes) that may interfere with subsequent steps of the method of the invention. Separation of the DNA minicircle from other components may be achieved using any suitable means.

In some embodiments, separation may involve physical separation, e.g. using electrophoresis or chromatography methods, and subsequent isolation of the DNA minicircle.

In some embodiments, separation may involve degradation and/or denaturation of components. For instance, as discussed further below, the parental minicircle plasmid may contain cleavage domains (e.g. nickase cleavage domains) that enable the plasmid backbone and unreacted (unrecombined) parental minicircle plasmid to be degraded or cleaved (e.g. by a cleavage enzyme such as a nickase) following the recombination reaction, such that they cannot function as RCA templates. Thus, in some embodiments, separation of the DNA minicircle from other components (e.g. plasmid backbone and unreacted (unrecombined) parental minicircle plasmid) may involve a step of contacting the components with a cleavage enzyme under conditions that result in cleavage of the components. It will be evident that this contacting step may be in vivo (e.g. in the host cell by inducing the expression of a cleavage enzyme) or in vitro (e.g. by contacting the components, e.g. host cell lysate or products of the in vitro recombination reaction, with a cleavage enzyme). The product of the degradation (e.g. cleavage) step may be further purified to isolate the DNA minicircle.

Thus, in some embodiments, the step of isolating the DNA minicircle comprises separating or purifying the DNA minicircle from other components, particularly other nucleic acid components or molecules (e.g. plasmid backbone and unreacted (unrecombined) parental minicircle plasmid). This isolation, separation or purification may be done by any suitable methods known in the art.

In some embodiments, following the isolation step (e.g. separation and purification step), the DNA minicircle may be substantially free of any contaminating components derived from the materials or components used in the step of obtaining or preparing the DNA minicircle (e.g. nucleic acid components and/or degradation products). In some embodiments, the DNA minicircles are purified to a degree of purity of more than about 50 or 60%, e.g. more than about 70, 80 or 90%, such as more than about 95 or 99% purity as assessed w/w (dry weight). Such purity levels may include degradation products of the DNA minicircles.

It is not essential to separate the DNA minicircles from other components (e.g. plasmid backbone and unreacted (unrecombined) parental minicircle plasmid) before performing other steps of the invention. As discussed below, providing a RCA primer that hybridises only to the DNA minicircle may ensure that only the DNA minicircle functions as a template in the RCA reaction. Alternatively, once the plasmid backbone and unreacted (unrecombined) parental minicircle plasmid has been degraded (e.g. cleaved such that they cannot function as a template for RCA), it is not necessary to separate the DNA minicircle from the degradation products (although separation may be preferred in some embodiments).

Thus, in some embodiments, it may be useful to prepare enriched preparations of the DNA minicircles which have lower purity, e.g. contain less than about 50% of the DNA minicircles, e.g. less than about 40%, 30%, 20%, 10%, 5% or less as assessed w/w (dry weight).

In a further aspect, the present invention provides a parental minicircle plasmid comprising a polynucleotide sequence bordered by cleavage domains (i.e. a pseudogene as defined herein), bordered by recombinase attachment sites. In some embodiments, the cleavage domains comprise or consist of a sequence capable of forming a hairpin structure as defined below. In some embodiments, the parental minicircle encodes a recombinase enzyme as defined above, e.g. PhiC31 integrase, ParA resolvase or FLP recombinase.

Thus, in some embodiments the invention provides a parental minicircle plasmid comprising:

(a) a first domain comprising: (i) a polynucleotide sequence bordered by cleavage domains (i.e. a pseudogene as defined herein, e.g. comprising hairpin cleavage domains) which is bordered by recombinase attachment sites; or (ii) an insertion site for a polynucleotide sequence bordered by cleavage domains, which is bordered by recombinase attachment sites; and

(b) a second domain encoding a recombinase enzyme (i.e. a recombinase enzyme that recognises the recombinase attachment sites of the first domain), optionally under the control of an inducible promoter, e.g. an arabinose inducible promoter.

In some embodiments, the parental minicircle plasmid of the invention encodes a PhiC31 integrase and contains recombinase attachment sites for PhiC31 integrase. Thus, in some embodiments, the recombinase attachment sites comprise nucleotide sequences as set forth in SEQ ID NO: 4 and SEQ ID NO: 5.

In some embodiments, the parental minicircle plasmid of the invention encodes ParA resolvase and contains recombinase attachment sites for ParA resolvase. Thus, in some embodiments, the recombinase attachment sites comprise nucleotide sequences as set forth in SEQ ID NO: 29 and SEQ ID NO: 30.

Thus, in some embodiments, the parental minicircle plasmid of the invention comprises a nucleotide sequence as set forth in SEQ ID NO: 28 or a nucleotide sequence with at least 80% sequence identity to a sequence as set forth in SEQ ID NO: 28, wherein the plasmid contains the functional domains described above, e.g. insertion site, attachment sites, recombinase encoding sequence, and one or more of the functional domains described in detail below, e.g. origin of replication, selection sequence, nickase cleavage domains etc.

In some embodiments, the parental minicircle plasmid of the invention encodes FLP recombinase and contains recombinase attachment sites for FLP recombinase. Thus, in some embodiments, the recombinase attachment sites comprise nucleotide sequences as set forth in SEQ ID NO: 31 and SEQ ID NO: 32.

Thus, in some embodiments, the parental minicircle plasmid of the invention comprises a nucleotide sequence as set forth in SEQ ID NO: 27 or a nucleotide sequence with at least 80% sequence identity to a sequence as set forth in SEQ ID NO: 27, wherein the plasmid contains the functional domains described above, e.g. insertion site, attachment sites, recombinase encoding sequence, and one or more of the functional domains described in detail below, e.g. origin of replication, selection sequence, nickase cleavage domains etc.

In a further embodiment the invention provides a parental minicircle plasmid comprising:

(a) a first domain comprising: (i) a polynucleotide sequence bordered by cleavage domains (i.e. a pseudogene as defined herein, e.g. comprising hairpin cleavage domains) which is bordered by recombinase attachment sites; or (ii) an insertion site for a polynucleotide sequence bordered by cleavage domains, which is bordered by recombinase attachment sites; and

(b) a second domain comprising two or more nickase cleavage domains, wherein each strand of the plasmid DNA comprises at least one nickase cleavage domain (i.e. such that contacting the plasmid with a nickase capable of cleaving the cleavage domains results in cleavage of both strands of the plasmid).

In some embodiments, the second domain comprises 3, 4, 5, 6, 7, 8, 9, 10 or more, such as 15, 20 or 25 or more nickase cleavage domains, wherein each strand of the plasmid DNA comprises at least one nickase cleavage domain. In some embodiments, the second domain comprises 4-8, e.g. 6 nickase cleavage domains, wherein each strand of the plasmid DNA comprises at least one nickase cleavage domain. In some embodiments, the nickase cleavage domains are configured such that each strand is cleaved multiple times, e.g. where the plasmid contains 6 nickase cleavage domains, they are configured such that each strand is cleaved 3 times.

In some embodiments, at least one (e.g. 2, 3 or 4) of the nickase cleavage domains (the cleavage domain recognition sequence) is in close proximity to one of the attachment sites, e.g. within 150 nucleotides of one of the attachment sites, such as within 120, 110, 100, 90, 80, 70, 60, 50, 40 or 30 nucleotides of one of the attachment sites. For instance, where the sequence of the plasmid is depicted as a linear molecule (e.g. SEQ ID NO: 1), close proximity refers to a sequence within 150 nucleotides (as defined above) of the start (5′ end) of the first attachment sequence (e.g. attB) or end (3′ end) of the second attachment site sequence (e.g. attP). In some embodiments, the plasmid contains two nickase cleavage domains in close proximity to each attachment site, i.e. two upstream of (5′ to) the first attachment site and two downstream of (3′ to) the second attachment site. In some embodiments, two of the nickase cleavage domains are within about 50 (e.g. about 40) nucleotides of an attachment site (e.g. upstream), preferably wherein the domains are configured to enable cleavage of both strands. Additionally or alternatively, in some embodiments, two of the nickase cleavage domains are within about 120 (e.g. about 110) nucleotides of an attachment site (e.g. downstream), preferably wherein the domains are configured to enable cleavage of both strands.

Cleavage domains for any suitable nickase may be included in the plasmid. In some embodiments, the cleavage domains are for nickase enzymes Nt.BspQI or Nb.BsrDI, or a combination thereof. In some embodiments, the cleavage domains for nickase Nt.BspQI may have the sequence GCTCTTC (SEQ ID NO: 25). In some embodiments, the cleavage domains for nickase Nb.BsrDI may have the sequence GCAATG (SEQ ID NO: 26).

In preferred embodiments, the plasmid may contain 2, 3 or more different nickase cleavage domains. For instance, in some embodiments a different nickase is used to cleave each strand, i.e. the two or more nickase cleavage domains contain sequences recognised by different nickase enzymes, e.g. the combination of enzymes mentioned above. Thus, in some embodiments, cleavage may utilise a mixture of nickase enzymes. However, in some embodiments, all of the nickase cleavage domains are the same, i.e. the substrate for the same nickase enzyme. This enables cleavage (i.e. degradation or destruction) of intact parental minicircle plasmid and backbone plasmid with a single nickase enzyme.

In some embodiments, the plasmid may comprise at least one pair of nickase cleavage domains in close proximity to one another, such as within 50 nucleotides or fewer, such as 40, 30, 20, 15, 10 or 5 nucleotides or fewer, from one another. In some embodiments, the plasmid may comprise 2, 3, 4, 5, 6, 7, 8, 9, 10 or more such nickase cleavage domain pairs. In some embodiments, the nickase cleavage domains within each pair may be different from each other. In some embodiments, the at least one pair of nickase cleavage domains (e.g. 2 pairs) satisfy the requirements above relating to proximity to the attachment sites, i.e. both cleavage domains within the pair are in close proximity to an attachment site.

In some embodiments, the first domain of the parental minicircle plasmid comprises a nickase cleavage domain, e.g. located between a cleavage domain and a recombinase attachment site, i.e. such that a recombinase attachment site indirectly borders the polynucleotide sequence bordered by cleavage domains. In some embodiments, the nickase cleavage domain in the first domain is the same as at least one of the nickase cleavage domains in the second domain. In some embodiments, all of the nickase cleavage domains in the parental minicircle plasmid are the same.

Cleavage domains in the first domain of the minicircle plasmid may be termed “first domain cleavage domains” and cleavage domains in the second domain of the minicircle plasmid may be termed “second domain cleavage domains”. Thus, the first domain of the minicircle plasmid may contain additional cleavage domains to the cleavage domains that border the polynucleotide sequence, i.e. the sequence encoding polynucleotide to be produced by the method, e.g. a nickase cleavage domain.

In some embodiments, the second domain of the parental minicircle plasmid may comprise one or more endonuclease (e.g. homing endonuclease) cleavage domains as defined above, e.g. 2, 3, 4, 5, 6, 7, 8, 9, 10 or more. The one or more endonuclease (e.g. homing endonuclease) cleavage domains may be in addition or as an alternative to the nickase cleavage domains. In some embodiments, the endonuclease (e.g. homing endonuclease) cleavage domains are for I-Scel.

In some embodiments, the second domain of the parental minicircle plasmid may comprise a sequence encoding a recombinase as defined above.

In some embodiments, the second domain of the parental minicircle plasmid may comprise a sequence encoding an endonuclease (e.g. a homing endonuclease) as defined above.

The “insertion site” in the first domain of the parental minicircle plasmid refers to a site into which a polynucleotide sequence bordered by cleavage domains may be introduced, e.g. a multiple cloning site or polylinker that contains a plurality (e.g. 2-20, 2-15 or 2-10) multiple unique restriction enzyme cleavage sites. In some embodiments, the insertion site may comprise a nickase cleavage domain as defined above. In some embodiments, the nickase cleavage site, when present, may be provided as part of the pseudogene to be inserted into the parental minicircle plasmid.

The term “recombinase attachment sites” refers to short DNA sequences (e.g. about 30-150 bp, such as about 40-60 bp) endogenously located in genomes, e.g. in phage (phage attachment site, attP) and bacterial (bacterial attachment site, attB) genomes that are recognised by recombinase enzymes. Site-specific recombinases (SSRs) perform rearrangements of DNA segments by recognizing and binding to the attachment sites, at which they cleave the DNA backbone, exchange the two DNA helices involved, and rejoin the DNA strands. The parental minicircle plasmid of the invention may contain any suitable recombinase attachment sites. In some preferred embodiments, the parental minicircle plasmid of the invention contains recombinase attachment sites for PhiC31 integrase. Thus, in some embodiments, the recombinase attachment sites comprise nucleotide sequences as set forth in SEQ ID NO: 4 and SEQ ID NO: 5. In some embodiments, the parental minicircle plasmid of the invention contains recombinase attachment sites for ParA resolvase. Thus, in some embodiments, the recombinase attachment sites comprise nucleotide sequences as set forth in SEQ ID NO: 29 and SEQ ID NO: 30. In some embodiments, the parental minicircle plasmid of the invention contains recombinase attachment sites for FLP recombinase. Thus, in some embodiments, the recombinase attachment sites comprise nucleotide sequences as set forth in SEQ ID NO: 31 and SEQ ID NO: 32.

It will be understood that the second domain of the parental minicircle plasmid also contains the sequences that are required for the propagation of the parental minicircle plasmid itself, such as an origin of replication. Such sequences are well-known in the art and any suitable sequence may be selected for use in the plasmid (e.g. a ColE1 sequence). In some embodiments, the second domain of the parental minicircle plasmid may comprise a sequence which allows for the selection of host cells comprising the plasmid. Sequences suitable for this purpose are well-known in the art, and any such appropriate sequence may be used. In some embodiments, the second domain of the parental minicircle plasmid may comprise an antibiotic resistance gene. In some embodiments, the antibiotic resistance gene may be a kanamycin or ampicillin resistance gene.

Thus, in some embodiments, the parental minicircle plasmid of the invention comprises a nucleotide sequence as set forth in SEQ ID NO: 1 or a nucleotide sequence with at least 80% sequence identity to a sequence as set forth in SEQ ID NO: 1, wherein the plasmid contains the functional domains described above, e.g. insertion site, attachment sites, nickase cleavage domains, origin of replication, selection sequence etc.

Nucleic acid sequence identity may be determined by, e.g. FASTA Search using GCG packages, with default values and a variable pamfactor, and gap creation penalty set at 12.0 and gap extension penalty set at 4.0 with a window of 6 nucleotides. Preferably said comparison is made over the full length of the sequence, but may be made over a smaller window of comparison, e.g. less than 600, 500, 400, 300, 200, 100 or 50 contiguous nucleotides.

The term “operably linked” refers to a functional linkage between two or more elements. For example, an operable linkage between a polynucleotide encoding a protein (e.g. an enzyme, such as a recombinase or endonuclease) and a regulatory sequence (i.e. a promoter) is functional link that allows for the expression of the polynucleotide encoding the protein. Operably linked elements may be contiguous or non-contiguous.

The present invention advantageously may be used to produce polynucleotides comprising any sequence. Thus, any suitable sequence may be used as the polynucleotide sequence in the DNA minicircle of the invention. By a suitable sequence, it is meant that the polynucleotide sequence domain should not interfere with (i.e. inhibit or distort) the production or cleavage of the RCA product. For instance, in some embodiments the polynucleotide sequence may be designed to avoid the generation of secondary structures that may inhibit the progression of, or result in the displacement of, the polymerase performing the RCA reaction. Nevertheless, in some embodiments, the polynucleotide sequence may be, or may encode, an aptamer.

Moreover, the polynucleotide sequence may be designed such that it does not hybridize specifically to the cleavage domains in the RCA product. Alternatively viewed, the cleavage domains that border the polynucleotide sequence may be designed such that they do not hybridize specifically to the polynucleotide sequence(s) in the RCA product.

In embodiments where the DNA minicircle comprises a plurality of polynucleotide sequences, each sequence may be designed such that it does not hybridize specifically to the other polynucleotide sequences in the RCA product. However, in some embodiments, it may be desirable to produce polynucleotides containing regions of complementarity, e.g. to enable said polynucleotides to interact, particularly following their release from the RCA product. Thus, in some embodiments, the polynucleotide sequences may be designed to facilitate the interaction (e.g. hybridisation) of polynucleotides produced by the method, insofar as such interactions do not interfere with the production or cleavage of the RCA product, i.e. the production of the polynucleotides.

Thus, in some embodiments, the nucleic acid sequence of the polynucleotide sequence of the DNA minicircle has less than 80% sequence identity to the nucleic acid sequences in the cleavage domain and/or other polynucleotide sequences in the DNA minicircle. Preferably, the polynucleotide sequence of the DNA minicircle has less than 70%, 60%, 50% or less than 40% sequence identity to the nucleic acid sequences in the cleavage domain and/or other polynucleotide sequences in the DNA minicircle. Sequence identity may be determined by any appropriate method known in the art, e.g. the using BLAST alignment algorithm.

Thus, term “polynucleotide sequence” is not particularly limiting and refers to the template sequence used to produce the single stranded DNA polynucleotides of the invention, or a complement thereof. In this respect, the DNA minicircle obtained from the parental minicircle plasmid is double stranded and therefore contains both the repeated sequence in the RCA product obtained in step (b) and its reverse complement. Accordingly, the method comprises a step of processing the DNA minicircle to provide an RCA template. The processing step comprises a step of cleaving the strand of the DNA minicircle containing the sequence that will be repeated in the RCA product. In some embodiments, cleavage provides both the RCA template and the primer for the RCA reaction. In some embodiments, the cleaved strand may be separated from the uncleaved RCA template strand, e.g. by denaturation and/or degradation of the cleaved strand. Denaturation and/or degradation may be achieved by any suitable means known in the art, e.g. heat, alkali. It may not be necessary to fully denature or degraded the cleaved strand of the DNA minicircle in order to provide an RCA template, e.g. partial denaturation and/or degradation such that a primer can be hybridised may be sufficient.

The present invention may be used to produce single stranded DNA polynucleotides of any desired length. As discussed above, and as shown in the Examples, the present method advantageously may be used to produce single stranded DNA polynucleotides that are about 1 kb or more in length. Alternatively viewed, the present method is particularly advantageous when the pseudogene is about 1 kb or more in length. As the pseudogene may comprise more than one polynucleotide sequence bordered by cleavage domains, the method is equally applicable for the production of shorter polynucleotides. However, where the method is used for the production of shorter polynucleotides, i.e. polynucleotides of about 0.5 kb or less, it is preferred that pseudogene encodes more than one polynucleotide sequence to be produced.

Thus, in some embodiments, the DNA minicircle obtained from the parental minicircle plasmid is at least about 0.5 kb in length, such as at least about 0.6, 0.7, 0.8, 0.9, 1.0, 1.2, 1.5 kb in length. In some embodiments, the DNA minicircle obtained from the parental minicircle plasmid is at least about 2 kb in length, e.g. 3, 4, 5, 6, 7, 8, 9 or 10 kb in length. For instance, the DNA minicircle obtained from the parental minicircle plasmid may be about 1-100 kb, e.g. about 1-50 kb, 2-45 kb, 3-40 kb, 4-35 kb or 5-30 kb. It will be evident that the size of the DNA minicircle will be dependent on the size and number of different polynucleotides to be produced by the method as defined below.

Accordingly, a polynucleotide sequence produced by the method may be between about 6 to about 50000 nucleotides in length. Thus, in some embodiments, the method may be viewed as the production of oligonucleotides. In this respect, the boundary between the size of a “polynucleotide” and an “oligonucleotide” is not well-defined in the art. For instance, a sequence of fewer than 400 nucleotides may be termed an oligonucleotide. Accordingly, the terms polynucleotide and oligonucleotide are used interchangeably herein to refer to nucleotide sequences within the size range specified above. However, where the term “polynucleotide” is used, it will typically refer to sequences containing more than 400 nucleotides. Thus, in some embodiments, the method and use may be viewed as producing a plurality of single stranded DNA molecules.

In some embodiments, the polynucleotide sequence may be from about 50 to 50000 nucleotides in length, including from about 100 to about 25000 nucleotides in length, e.g. from about 100 to about 10000 nucleotides in length, from about 100 to about 8000 nucleotides in length, e.g., from about 200 to about 7500 nucleotides in length, such as from about 300 to about 6000 nucleotides in length, from about 400 to about 5000 nucleotides in length, from about 500 to about 4000 nucleotides in length, from about 500 to about 3000 nucleotides in length, from about 500 to about 2500 nucleotides in length, from about 600 to about 2000 nucleotides in length, from about 600 to about 1500 nucleotides in length, from about 750 to about 1250 nucleotides in length, from about 1000 to about 1250 nucleotides in length, and so on.

As noted above, the invention is particularly effective in the production of longer polynucleotides, e.g. comprising about 800 or more nucleotides, such as about 900, 1000, 1100, 1200, 1300, 1400, 1500, 1600, 1700, 1800, 1900, 2000 or more nucleotides. For instance, the polynucleotides produced by the invention may contain about 1000-50000, 1000-40000, 1000-30000, 1000-20000, 1000-10000, 1000-9000, 1000-8000, 1000-7000, 1000-6000, 1000-5000 or 1000-4000 nucleotides, e.g. comprising about 2500, 3000, 3500 or more nucleotides.

In some embodiments, the DNA minicircle comprises a plurality of polynucleotide sequences, wherein each polynucleotide sequence is bordered by cleavage domains. The polynucleotide sequences may be the same or different from each other, or a combination thereof. Thus, in some embodiments, the DNA minicircle may comprise more than one copy of the same polynucleotide sequence, e.g. 2, 3, 4, 5 etc. copies, as defined below. In some embodiments, the DNA minicircle may comprise one copy each of a plurality of different polynucleotide sequences, e.g. 2, 3, 4, 5 etc. different polynucleotide sequences, as defined below. In some embodiments, the DNA minicircle may comprise one or more copies of a plurality of different polynucleotide sequences. As discussed further below, the present invention may be used to generate a plurality of polynucleotides in controlled stoichiometry based on the number of copies of polynucleotide sequences in the DNA minicircle.

It will be evident that the plurality of polynucleotide sequences in the DNA minicircle may be present in any order. For instance, multiple copies of the same polynucleotide sequence may be directly adjacent to each other in the DNA minicircle (separated only by the cleavage domains that border the polynucleotide sequences). Alternatively, multiple copies of the same polynucleotide sequence may be interspersed with different polynucleotide sequences. The DNA minicircle (i.e. the pseudogene in the parental minicircle plasmid that results in the DNA minicircle) may be designed (e.g. the order of the plurality of polynucleotide sequences) to avoid or minimise interactions between the repeated sequences in the RCA product that may interfere with the production or cleavage of the RCA product as defined above.

As used herein, the term “plurality” means two or more, e.g. at least 2, 3, 4, 5, 6, 7, 8, 9, 10, 20 or 30 or more, such as 50, 100, 150, 200, 250 or more depending on the context of the invention. For instance, the DNA minicircle may contain at least 2, 3, 4, 5, 6, 7, 8, 9, 10, 20 or 30 or more polynucleotide sequences, such as 2-100, 3-90, 4-80, 5-70, 6-60, 7-50, 8-40, 9-30 or 10-20 polynucleotide sequences. In some embodiments, the method of the invention may produce at least 2, 3, 4, 5, 6, 7, 8, 9, 10, 20 or 30 or more single stranded DNA polynucleotides, such as 50, 100, 150, 200, 250 or more, i.e. polynucleotides with different sequences and/or structures. It can be seen that if the DNA minicircle contains a plurality of different polynucleotide sequences, each template will result in a plurality of different single stranded DNA polynucleotides. Furthermore, as an RCA reaction may utilise a plurality of DNA minicircles, the reaction will result in a plurality of each single stranded DNA polynucleotide, e.g. 10³, 10⁴, 10⁵, 10⁶, 10⁷, 10⁸, 10⁹, 10¹⁰ or more copies of each single stranded DNA polynucleotide.

The term “different” refers to polynucleotide sequences or single stranded DNA polynucleotides comprising one or more different nucleotides. Thus, different polynucleotide sequences may differ by one or more, e.g. 2, 3, 4, 5, 6, 7, 8, 9, 10 or more, nucleotides, e.g. 20, 30, 40, 50, 60, 70, 80, 90 or more nucleotides. The differences may be in the length and/or sequence of the polynucleotide sequence. Alternatively viewed, the different polynucleotide sequences have less than 100% sequence identity each other, such as less than 99%, 98%, 97%, 96%, 95%, 90%, 85%, 80%, 70%, 60%, 50% to each other.

The present invention may therefore be used to produce a number of different single stranded DNA polynucleotides simultaneously. In particular, by altering the sequence of the DNA minicircle, specifically, by controlling the number of copies of each different polynucleotide sequence that are present, it is possible to control the stoichiometry of the single stranded DNA polynucleotides that are ultimately produced by the present method.

The term “cleavage domain” as used herein typically refers to a domain within the parental minicircle plasmid and/or DNA minicircle that facilitates cleavage of the plasmid and/or minicircle (e.g. within or in proximity to the cleavage domain) or results in a domain within the RCA product that can be cleaved specifically to release the single stranded DNA polynucleotides. Thus, the cleavage domain in the parental minicircle plasmid and/or DNA minicircle may be capable of cleavage directly (e.g. an endonuclease recognition site that results in cleavage within or in proximity to the cleavage domain upon contact with a compatible endonuclease under suitable conditions) or it may simply encode a cleavage domain that is only functional in the RCA product or functional under specific conditions, e.g. upon contact with a co-factor.

In preferred embodiments, cleavage domains in the second domain of the parental minicircle plasmid are capable of cleavage directly, i.e. they direct endonuclease-mediated single or double stranded cleavage within or in proximity to the cleavage domain. Similarly, the nickase cleavage domain in the first domain of the parental minicircle plasmid (i.e. in the DNA minicircle) is capable of cleavage directly, i.e. it directs nicking endonuclease-mediated single stranded cleavage within or in proximity to the cleavage domain.

In some embodiments, cleavage domains that border the polynucleotide to be produced (i.e. cleavage domains in the pseudogene) encode a cleavage domain that is only functional in the RCA product or functional under specific conditions, e.g. upon contact with a co-factor.

“Cleavage” includes any means of breaking a covalent bond. Thus, in the context of the invention, cleavage involves cleavage of a covalent bond in a nucleotide chain (i.e. strand cleavage or strand scission), for example by cleavage of a phosphodiester bond.

Thus, in some embodiments, a cleavage domain may comprise a sequence that is recognised by one or more enzymes capable of cleaving a nucleic acid molecule, i.e. capable of breaking the phosphodiester linkage between two or more nucleotides. For instance, a cleavage domain may comprise a restriction endonuclease (restriction enzyme) recognition sequence. Restriction enzymes cut double-stranded or single stranded DNA at specific recognition nucleotide sequences known as restriction sites, and suitable enzymes are well-known in the art. For example, it may be particularly advantageous to use rare-cutting restriction enzymes, i.e. enzymes with a long recognition site (at least 8 base pairs in length), to facilitate the design of the polynucleotide sequence(s) in the DNA minicircle, e.g. to avoid the inclusion of a cleavage recognition site that occurs within the polynucleotide sequence(s).

In some embodiments, a cleavage domain (particularly a cleavage domain in the DNA minicircle, i.e. that borders the polynucleotide) may comprise a sequence that is recognised by a type II restriction endonuclease, more preferably a type IIs restriction endonuclease. While any suitable cleavage domain and cleavage enzyme may be used in the invention, in some embodiments the cleavage enzyme that recognises the cleavage domains bordering the polynucleotide sequences may be BseGI, BtsCI or an isoschizomer thereof, e.g. BstF5I or FokI. Other representative enzymes that may be used include BsrDI, BtsI, BtslMutI, MlyI or isoschizomers thereof.

As discussed above, the method finds particular utility in the production of longer single stranded polynucleotides, e.g. at least about 800 nucleotides in length. It will be understood that longer sequences are more likely to contain sequences recognised by endonucleases, particularly type IIs restriction endonucleases. Thus, in some embodiments, the cleavage domains that border the polynucleotide may be homing endonuclease cleavage domains as defined above.

Thus, in some embodiments, the cleavage enzyme used in the cleavage step may be homing endonucleases as defined above. In some embodiments, the cleavage domains that border the polynucleotide may be meganuclease cleavage domains. Thus, in some embodiments, the cleavage enzyme used in the cleavage step may be a meganuclease.

The term “meganuclease” refers to endonucleases characterized by a large recognition site, e.g. double-stranded DNA sequences of 12 to 40 base pairs. Thus, many homing endonucleases, such as I-Scel, may be viewed as meganucleases. Chimeric meganucleases may be produced by fusing a nucleic acid binding domain and an endonuclease cleavage domain from different proteins. For instance, any protein domain capable of site-specific recognition (binding) of a DNA sequence as described above may be fused to a cleavage domain from an endonuclease that cleaves outside of the sequence recognised by the endonuclease, e.g. at a specific distance from the recognition sequence. Any suitable meganuclease known in the art may be used in the methods described herein, e.g. in the step of cleaving the RCA product.

Thus, in some embodiments, the step of cleaving the product of the RCA reaction comprises contacting the RCA product with a cleavage enzyme under suitable conditions to selectively cleave the RCA product in the cleavage domains.

In some embodiments, a cleavage domain may be made functional (may be activated) in the RCA product by the addition of another component, i.e. the RCA product may be engineered to comprise a functional cleavage domain, e.g. an endonuclease recognition sequence. For example, this may be achieved by hybridising an oligonucleotide (termed herein a “restriction oligonucleotide” or “cleavage oligonucleotide”) to the cleavage domains of the RCA product to form a duplex. At least part of the formed duplex will comprise an endonuclease recognition site, which can be cleaved resulting in the release of the single stranded DNA polynucleotides. This may be particularly advantageous in embodiments where the nucleotides incorporated into the RCA product (e.g. functionalised nucleotides discussed below), particularly in the cleavage domains, may interfere with the activity (e.g. reduce the efficiency) of the cleavage enzyme.

Thus, in some embodiments, the step of cleaving the product of the RCA reaction may comprise contacting the RCA product with a cleavage oligonucleotide and a cleavage enzyme. The cleavage oligonucleotide and cleavage enzyme may be contacted with the RCA product simultaneously or sequentially.

In some embodiments, a cleavage domain may be cleaved by means other than a cleavage enzyme. For example, a cleavage domain may comprise a self-cleaving oligonucleotide sequence, such as a DNAzyme nuclease. Suitable self-cleaving sequences are known in the art. In embodiments that utilise a self-cleaving sequence, the cleavage domain may only be functional (active) in the RCA product. Alternatively, the self-cleaving sequence may be functional under specific conditions and thus the method may comprise a step of subjecting the RCA product to conditions that facilitate cleavage of the RCA product in the cleavage domains, i.e. conditions that activate the self-cleaving sequence, e.g. contacting the RCA product with a co-factor, such as metal ions, that are required for the self-cleaving activity. Alternatively viewed, the step of cleaving the product of the RCA reaction may comprise subjecting the RCA product to conditions that facilitate cleavage of the RCA product in the cleavage domains, i.e. conditions that activate the self-cleaving sequence, e.g. contacting the RCA product with a co-factor, such as metal ions, that are required for the self-cleaving activity.

In some embodiments, a cleavage domain comprises or consists of a sequence capable of forming a hairpin structure. A hairpin structure may also be known as a hairpin-loop or a stem-loop and these terms are used interchangeably herein. A hairpin is an intramolecular base-pairing pattern that can occur in a single-stranded DNA or RNA molecule. A hairpin occurs when two regions of the same strand, usually complementary in nucleotide sequence when read in opposite directions, base-pair (hybridise) to form a double stranded stem (a duplex) and an unpaired, i.e. single-stranded, loop. The resulting structure can be described as lollipop-shaped.

Thus, in some embodiments, where the cleavage domain comprises or consists of a sequence capable of forming a hairpin structure, the cleavage domain comprises sequences that are self-complementary. As the RCA product extends, the hybridization of these self-complementary regions results in a hairpin structures, wherein the double-stranded portion of the hairpin structure comprises a sequence that is recognised by a cleavage enzyme. Thus, cleavage of the double-stranded portion of the hairpin structures in the RCA product results in the release of the single stranded DNA polynucleotides and hairpin structures (i.e. oligonucleotides that form the hairpin structures).

The term “hybridisation” or “hybridises” as used herein refers to the formation of a duplex between nucleotide sequences which are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T(U), G and C of one sequence is then aligned with a T(U), A, C and G, respectively, of the other sequence. RNA sequences can also include complementary G=U or U=G base pairs. Thus, two sequences need not have perfect homology to be “complementary” under the invention. Usually two sequences are sufficiently complementary when at least about 90% (preferably at least about 95%) of the nucleotides share base pair organization over a defined length of the molecule.

Upon cleavage of the RCA product, the single stranded DNA polynucleotides are released. The polynucleotides that are released may consist only of the polynucleotide sequence (i.e. with no additional nucleotides), or they may comprise one or more additional nucleotides from the cleavage domains that border the polynucleotide sequences at one or both ends. Thus in some embodiments, the single stranded DNA polynucleotides may comprise 1, 2, 3, 4, 5, 6, 7, 8, 9 or 10 or more nucleotides from the cleavage domains at one or both ends. Preferably, the sequence of the DNA minicircle is designed such that when the RCA product is cleaved, the polynucleotides that are released do not contain any additional nucleotides from the cleavage domains. Alternatively viewed, the cleavage domains that border the polynucleotide sequence(s) may be arranged in the DNA minicircle such that their cleavage in the RCA product results in the release of polynucleotides that do not contain any additional nucleotides from the cleavage domains.

As cleavage enzymes may cleave a nucleic acid molecule at a position outside of the cleavage enzyme recognition sequence, there may be one or more nucleotides between a cleavage domain and a polynucleotide sequence.

Alternatively viewed, the cleavage domain may contain nucleotide sequences in addition to the cleavage enzyme recognition sequence to ensure that cleavage results in the release of the complete single stranded DNA polynucleotides, preferably without any additional nucleotides (e.g. nucleotides that form part of the cleavage domains).

Thus, the term “bordered”, in the context of a polynucleotide sequence bordered by cleavage domains, refers to cleavage domains that are directly or indirectly adjacent to the polynucleotide sequence. Alternatively viewed, the cleavage domains are positioned at either end of the polynucleotide sequence, i.e. the cleavage domains are upstream and downstream (at the 5′ and 3′ ends) of the polynucleotide sequence. In some embodiments, the cleavage site of the cleavage domains (e.g. the site at which a cleavage enzyme cleaves a cleavage domain) is directly adjacent to the end of the polynucleotide sequence it borders. In some embodiments, the polynucleotide sequence and cleavage domain sequence may overlap, i.e. the end of the polynucleotide sequence may form part of the cleavage domain, e.g. when the cleavage site is an internal site within the cleavage domain, i.e. the cleavage domains may form the ends or part of the ends of the polynucleotide sequence. Thus, in some embodiments, there may be one or more, e.g. 1, 2, 3, 4, 5, 6, 7, 8, 9 or 10 or more, nucleotides between the cleavage domain and the polynucleotide sequence (i.e. between the ends of the sequences). In some embodiments, the cleavage domain and the polynucleotide sequence may overlap one or more, e.g. 1, 2, 3, 4, 5, 6, 7, 8, 9 or 10 or more, nucleotides. The size of the cleavage domains in the DNA minicircle is not particularly limited and will depend on the type of cleavage domain, as described above. It is preferred that the relative lengths of the polynucleotide sequences and the cleavage domains are designed or selected such that they are different from each other, to allow for the single stranded DNA polynucleotide sequences to be easily purified once the RCA product has been cleaved at the cleavage domain. The cleavage domain(s) may thus be selected to be shorter than the polynucleotide sequence(s) in the DNA minicircle. If the DNA minicircle contains a plurality of polynucleotide sequences of different lengths, the cleavage domain(s) may be selected to be shorter than the shortest polynucleotide sequence in the DNA minicircle. Alternatively, the cleavage domain(s) may be selected to be longer than the polynucleotide sequence(s) in the DNA minicircle. If the DNA minicircle contains a plurality of polynucleotide sequences of different lengths, the cleavage domain(s) may be selected to be longer than the longest polynucleotide sequence in the DNA minicircle, although this is less preferred. In some embodiments, the length of the cleavage domain(s) and the polynucleotide sequences differ by at least 2 nucleotides, such as at least 3, 4, 5, 6, 7, 8, 9 or 10 nucleotides. When the method is used to produce longer polynucleotides, the length of the cleavage domains and the polynucleotide sequences will differ considerably, e.g. by at least about 100, 150, 200, 250, 300 nucleotides.

In some embodiments, cleavage domains may be about 4-50 nucleotides in length, such as about 5-45, 6-40, 7-35 or 8-30 nucleotides in length. In some embodiments they may range from about 10 to 25 nucleotides in length, including from about 12 to 22 or from about 14 to 20. However, it will be evident that any suitable length of cleavage domain may be used in the invention as long as it meets the functional requirements described above.

The cleavage domains that border the polynucleotide sequences may be the same or different from each other. Advantageously, the cleavage domains that border the polynucleotide sequences are the same such that a single cleavage step is sufficient to release all of the single stranded DNA polynucleotides. However, as mentioned above, some cleavage enzymes may cleave a nucleic acid molecule at a position outside of the cleavage enzyme recognition sequence or may recognise more than one sequence (e.g. where variation within the enzyme recognition sequence is allowed). Thus, it is not necessary for the whole sequence of the cleavage domains to be the same in order for them to be cleaved by the same enzyme. For instance, in some embodiments, the step of cleaving the RCA product comprises contacting the RCA product with a single cleavage enzyme under conditions suitable to cleave the cleavage domains in the RCA product.

Suitable conditions to cleave the cleavage domains in the RCA product will be dependent on the means used to achieve cleavage. For instance, where cleavage is achieved using a cleavage enzyme, such as a restriction endonuclease or homing endonuclease, conditions will differ depending on the enzyme selected, and suitable conditions are well-known in the art, e.g. the cleavage step may follow the manufacturer's instructions. Similarly, where cleavage is achieved using a self-cleaving sequence, such as a DNAzyme, conditions appropriate for the particular sequence may be used. An example of a range suitable conditions that may be used in the cleavage step is set out below.

For instance, a cleavage enzyme, e.g. a restriction or homing endonuclease, may specifically bind to its cleavage recognition site and selectively (e.g. specifically) cleave the nucleic acid in a variety of buffers, such as phosphate buffered saline (PBS), 4-(2-hydroxyethyl)-1-piperazineethanesulfonic acid (HEPES), HEPES buffered saline (HBS), and Tris buffered saline (TBS), both with and without EDTA. Cleavage may occur in a pH range of about 3.0-10.0, e.g. 4.0-9.0, 5.0-8.0, over a wide range of temperatures, e.g. 0-70° C. The skilled person would readily be able to determine other suitable conditions.

The method of the invention comprises a step of performing rolling circle amplification using the DNA minicircle as a template. Rolling-circle amplification (RCA) is well known in the art, being described in Dean et al., 2001 (Rapid Amplification of Plasmid and Phage DNA Using Phi29 DNA Polymerase and Multiply-Primed Rolling Circle Amplification, Genome Research, 11, pp. 1095-1099), the disclosures of which are herein incorporated by reference. In brief, RCA relates to the synthesis of nucleic acid molecules using a circular single stranded nucleic acid molecule, e.g. a circle or circular oligonucleotide, as rolling circle template (a RCA template) and a strand-displacing polymerase to extend a primer which is hybridised to the template. The addition of a polymerase and nucleotides starts the synthesis reaction, i.e. polymerization. As the rolling circle template is endless, the resultant product is a long single stranded nucleic acid molecule composed of tandem repeats that are complementary to the rolling circle template.

A typical RCA reaction mixture includes a DNA minicircle acting as a template, and one or more primers that are employed in the primer extension reaction, e.g. RCA may be templated by a single primer to generate a single concatemeric product or multiple primers, each annealing to a different region of the circular template to produce multiple concatemeric products per circle. The oligonucleotide primers with which the circular nucleic acid may be contacted will be of sufficient length to provide for hybridization to the DNA minicircle under annealing conditions. In some embodiments, the primer may be provided by cleaving (e.g. nicking) a single strand of the double stranded DNA minicircle provided in step (a).

In addition to the above components, the reaction mixture used in the invention typically includes a polymerase (defined further below, e.g. phi29 DNA polymerase), one or more nucleotides and other components required for a DNA polymerase reaction as described below. The desired polymerase activity may be provided by one or more distinct polymerase enzymes.

In some embodiments, the nucleotides present in the reaction mixture are conventional nucleotides. The term “conventional nucleotides” as used herein refers to deoxynucleotides comprising one of the four bases found in DNA; adenine, guanine, cytosine and thymine. The term “conventional nucleotides” thus encompasses, for example, dATP, dGTP, dCTP and dTTP. Whilst uracil is not typically found in DNA naturally, dUTP readily may be used instead of, or in addition to, dTTP. Thus, in the context of the present invention, dUTP may be viewed as a “conventional” nucleotide. When the method is used to produce standard (unmodified) single stranded DNA polynucleotides, the reaction mixture will typically include all four different types of dNTPs corresponding to the four naturally occurring bases present, i.e. dATP, dTTP, dCTP and dGTP. However, as noted above, dUTP may be used instead of, or in addition to, dTTP. Moreover, in some embodiments, the reaction mixture may include five dNTPs, i.e. dATP, dCTP, dGTP, dTTP and dUTP. It will be understood that reaction mixture need only include the nucleotides present in the polynucleotide(s) to be produced by the method, i.e. in some embodiments the reaction mixture may contain three or fewer types of dNTPs. In the subject methods, each dNTP will typically be present in an amount ranging from about 10 to 5000 μM, usually from about 20 to 1000 μM. Each dNTP may be present in the different amounts or an equal amount of each dNTP may be used.

In some embodiments, the methods may be used to produce functionalised single stranded DNA polynucleotides. Thus, in some embodiments, the RCA reaction may be performed in the presence of one or more functionalised nucleotides, i.e. the reaction mixture may comprise one or more functionalised nucleotides. The terms “functionalised nucleotides” or “functionalised dNTPs” refer to nucleotides that comprise a modification relative to unmodified conventional nucleotides, wherein said modification provides said functionalised nucleotides and/or polynucleotides comprising at least one functionalised nucleotide with additional or alternative properties or characteristics, i.e. relative to the corresponding conventional nucleotide. For instance, the modification may render the nucleotide detectable, e.g. by the incorporation of a label, or capable of interacting and/or reacting with another component, i.e. a component with which the corresponding conventional nucleotide does not interact or react. In some embodiments, the modification may render the polynucleotide containing the nucleotide resistant to degradation, e.g. chemical and/or enzymatic degradation (e.g. nuclease degradation), or may alter the metabolism of the nucleotide.

Modified (e.g. functionalised) nucleotides have previously been used in enzymatic reactions to produce oligonucleotides, but only in the context of producing functionalised DNA via polymerase chain reactions (PCR) or primer extension (PE) reactions. These reactions necessitate the use of specific primers, which must then later be removed, and require not only that the modified nucleotides are incorporated by the polymerase, but also, in the case of PCR, that the functionalised products are recognised as templates. This can be problematic in the case of some modifications, and thus limits the utility of these methods. Moreover, the primers are synthesized by solid-phase methods and are not sequence verified, leading to the amplification of errors in the newly synthesized DNA. In addition, PCR-based methods result overwhelmingly in double stranded DNA products, and thus additional steps of elution and purification are necessary to obtain functionalised single stranded oligonucleotides.

The present inventors have found that functionalised nucleotides may be efficiently incorporated into the RCA product by DNA polymerases with strand displacement activity, and do not prevent the formation of hairpin structures or interfere with their stability or effective cleavage. Moreover, the inventors have also found that functionalised nucleotides may be utilised whilst still maintaining the beneficial characteristics associated with the present method, as the functionalised nucleotides are not required be recognised as a template for further amplification, which means that greater numbers of functionalised residues can be incorporated. Accordingly, the single stranded DNA polynucleotides produced by the method of the present invention may be functionalised polynucleotides, i.e. may comprise functionalised nucleotides. It will thus be understood that the terms “polynucleotides”, “single stranded polynucleotides”, and “single stranded DNA polynucleotides” as used herein in reference to polynucleotides produced by the method of the present invention may refer to polynucleotides comprising solely conventional nucleotides, or to polynucleotides comprising functionalised nucleotides.

It will be evident that when the functionalised nucleotides are used in combination with the equivalent conventional nucleotides, the functionalised nucleotides may be incorporated randomly in the concatemer produced by the RCA reaction and thus cleavage of the concatemer results in a plurality (e.g. library) of single stranded functionalised DNA polynucleotides, i.e. where the functionalised nucleotides are incorporated in different positions in the polynucleotides. It will be further evident that the diversity of the functionalised polynucleotides produced (i.e. the diversity of the library) may be increased by using a DNA minicircle containing a plurality of polynucleotide sequences, each bordered by cleavage domains. The polynucleotide sequences may differ in their sequence and/or length. Additionally or alternatively, the diversity of the functionalised polynucleotides may be increased by using a combination of functionalised nucleotides in the RCA reaction. Still further diversity may be introduced into the library of functionalised polynucleotides by modifying the polynucleotides after their synthesis, e.g. by conjugating molecules or components to the functionalised nucleotides in the polynucleotides.

Thus, in some embodiments, one or more of the conventional nucleotides (or a proportion thereof) may be substituted with an equivalent functionalised nucleotide. For instance, dATP may be substituted with dATP conjugated to a fluorophore, as described in more detail below. Thus, in some embodiments, the reaction mixture may contain three types of conventional nucleotides and one type of functionalised nucleotide.

The RCA reaction mixture may further include an aqueous buffer medium that includes a source of monovalent ions, a source of divalent cations and a buffering agent. Any convenient source of monovalent ions, such as KCl, K-acetate, NH₄-acetate, K-glutamate, NH₄Cl, ammonium sulphate, and the like may be employed. The divalent cation may be magnesium, manganese, zinc and the like, where the cation will typically be magnesium. Any convenient source of magnesium cation may be employed, including MgCl₂, Mg-acetate, and the like. The amount of Mg²⁺ present in the buffer may range from 0.5 to 10 mM, but will preferably range from about 3 to 6 mM, and will ideally be at about 5 mM. Representative buffering agents or salts that may be present in the buffer include Tris, Tricine, HEPES, MOPS and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5. Other agents which may be present in the buffer medium include chelating agents, such as EDTA, EGTA and the like.

The DNA minicircle that is provided in step a) of the method is a double stranded DNA molecule. Accordingly, the DNA minicircle must be processed in order to function as an RCA template. Thus, in some embodiments, step (b) of the method comprises cleaving a single strand of the DNA minicircle to provide an RCA template.

In embodiments in which a single strand of the double stranded DNA minicircle is cleaved (e.g. nicked) to provide the RCA template (and the primer for the RCA reaction), it may be useful to include a single stranded binding protein in the RCA reaction mixture. For example, E. coli single stranded DNA binding protein has been used to increase the yield and specificity of primer extension reactions and PCR reactions. (U.S. Pat. Nos. 5,449,603 and 5,534,407). The gene 32 protein (single stranded DNA binding protein) of phage T4 apparently improves the ability to amplify larger DNA fragments (Schwartz, et al., Nucl. Acids Res. 18: 1079 (1990)), it enhances DNA polymerase fidelity (Huang, DNA Cell. Biol. 15: 589-594 (1996)) and, most importantly, it prevents DNA polymerase from switching templates, e.g. to the already produced single stranded DNA, and synthesizing double stranded DNA (Ducani et al. Nucl. Acids Res. 42: 10596 (2014)). When employed, such a protein will be used to achieve a concentration in the reaction mixture that ranges from about 0.01 ng/μL to about 1 μg/μL; such as from about 0.1 ng/μL to about 100 ng/μL; including from about 1 ng/μL to about 10 ng/μL.

The RCA reaction ultimately produces a polynucleotide product comprising adjacent (tandem) repeats of the complementary sequence of the DNA minicircle. This product may be known as a concatemer, an RCA product or “RCP”. The RCA product therefore comprises a linear sequence made up of polynucleotide sequences (or more particularly the reverse complement of the polynucleotide sequences of the DNA minicircle template) bordered by cleavage domains.

As an alternative to the use of a nickase to generate a primer for the RCA reaction, the RCA reaction mixture may comprise one or more oligonucleotide primers, which initiate the RCA polymerisation reaction. The primers will be of sufficient length to provide for hybridization to DNA minicircle under annealing conditions. The primers will generally be at least 10 nucleotides in length, usually at least 12 nucleotides in length and more usually at least 14 nucleotides in length and may be as long as 30 nucleotides in length or longer, where the length of the primers will generally range from 14 to 50 nucleotides in length, usually from about 15 to 35 nucleotides in length.

The primers may anneal to any region within the DNA minicircle. In some embodiments, the DNA minicircle may comprise a specific domain (a RCA primer binding site) to which the primer may hybridise. In a representative embodiment, the DNA minicircle may comprise a sequence between the cleavage domains that border the polynucleotide sequence, which is not the polynucleotide sequence and which may function as the RCA primer binding site. The RCA primer binding site may be designed to be of a different length to the polynucleotide sequence, akin to the cleavage domains discussed above, to ensure that it can be readily separated from the single stranded DNA polynucleotides upon cleavage of the RCA product. It will be evident that in a DNA minicircle comprising a plurality of polynucleotide sequences bordered by cleavage domains, the RCA primer binding site may be between any two cleavage domains.

In a further representative embodiment, the DNA minicircle may comprise a RCA primer binding site between a cleavage domain and an attachment site (i.e. a junction sequence as defined below). Accordingly, the first domain of the parental minicircle plasmid may comprise a RCA primer binding site. The RCA primer binding site may be located directly or indirectly adjacent to an attachment site, such that the RCA primer binding site is retained in the DNA minicircle upon recombination of the parental minicircle plasmid.

As noted above, the DNA minicircle is produced by a recombination between the two recombinase attachment sites present in the parental minicircle plasmid. This recombination reaction involves a fusion of the attachment sites and thus results in the production of a so-called “junction sequence” at the site of the recombination. This junction sequence is present only in the DNA minicircle once it has undergone a successful recombination reaction, and is not present in the original parental minicircle plasmid or the backbone plasmid. This is particularly advantageous as it enables the RCA reaction to be performed without the need to separate the DNA minicircle from the unrecombined (intact) parental minicircle plasmid or the backbone plasmid.

Accordingly, in some embodiments, the primer for the RCA reaction may be designed to hybridise to the DNA minicircle at the junction sequence, so that the primer is only able to initiate the RCA reaction upon formation of the DNA minicircle, i.e. the primer specifically and selectively binds (hybridises) to the DNA minicircle. Alternatively viewed, the RCA primer does not bind to the un-recombined (intact) parental minicircle plasmid or the backbone plasmid. Alternatively put, the junction sequence may comprise the RCA primer binding site, or may form part of the RCA primer binding site. A primer that is capable of hybridising to the junction sequence in this manner may be referred to as a “bridging primer”.

The junction section will depend on the sequences of the attachment sites in the parental minicircle plasmid and the corresponding recombinase used to produce the DNA minicircle. In some embodiments, the junction sequence comprises or consists of a nucleotide sequence as set forth in SEQ ID NO: 2.

Thus, in some embodiments, the RCA primer comprises a nucleotide sequence capable of specifically and selectively binding (hybridising) to a nucleotide sequence as set forth in SEQ ID NO: 2, e.g. with the length and sequence identity characteristics defined elsewhere herein. In some embodiments, the RCA primer comprises a nucleotide sequence as set forth in SEQ ID NO: 3.

Thus, the DNA minicircle obtained from the parental minicircle plasmid contains a junction sequence formed by recombination of the attachment sites. In some embodiments, the DNA minicircle contains a junction sequence that comprises or consists of a nucleotide sequence as set forth in SEQ ID NO: 2.

Thus, the invention may also be seen to provide a DNA minicircle comprising a polynucleotide sequence to be produced bordered by cleavage domains (i.e. a pseudogene as defined herein) and a junction sequence, particularly a junction sequence that comprises or consists of a nucleotide sequence as set forth in SEQ ID NO: 2.

The term “annealing conditions” refers to the conditions under which two nucleic acid molecules comprising complementary nucleotide sequences will specifically hybridise to each other. Various parameters affect hybridisation including temperature, salt concentration, nucleic acid concentration, composition and length, and buffer composition. The skilled person readily can determine suitable annealing conditions for a particular primer/template combination for an RCA reaction as a matter of routine.

As noted above, the DNA minicircle is double stranded and the method comprises a step of cleaving a single strand of the DNA minicircle to provide an RCA template, before the RCA reaction is performed. It will be evident that the step of cleaving a single strand of the DNA minicircle may replace the step of providing a primer to initiate the RCA reaction, i.e. the cleaved strand functions as the RCA primer. Thus, alternatively viewed, step (b) comprises cleaving a single strand of the DNA minicircle to provide an RCA template and primer, i.e. the introduction of a single strand break in the DNA minicircle creates a 3′ end which can act as a primer for the RCA reaction. However, in some embodiments, it may be advantageous to provide one or more RCA primers in the reaction mixture in addition to cleaving one strand of the double stranded DNA minicircle, e.g. to increase the number of RCA products obtained per circle.

In some embodiments, the step of cleaving a single strand of the DNA minicircle to provide an RCA template comprises cleaving a single strand of the DNA minicircle with a cleavage enzyme. This cleavage of a single strand of the DNA minicircle results in a single strand break (a nick).

In some embodiments, it may be advantageous to cleave a single strand of the DNA minicircle multiple times in close proximity, in order to facilitate the binding of the DNA polymerase to the 3′ end generated by the cleavage. Accordingly, a single strand of the DNA minicircle may be cleaved two or more times, i.e. two or more nicks may be generated, in close proximity to each other. Preferably, the nicks are generated within 20 nucleotides, more preferably within 10 nucleotides, more preferably within 5 nucleotides of each other, e.g. within 1, 2, 3, 4 or 5 nucleotides of each other.

In some embodiments, the cleavage enzyme used to cleave one strand of the double stranded DNA minicircle is a nickase. Nickases are endonucleases which cleave only a single strand of a DNA duplex. Some nickases introduce single-stranded nicks only at particular sites on a DNA molecule, by binding to and recognizing a particular nucleotide recognition sequence. A number of naturally-occurring nickases have been discovered. Nickases are described in U.S. Pat. No. 6,867,028, which is herein incorporated by reference in its entirety and any suitable nickase may be used in the methods of the invention. In some embodiments, the cleavage enzyme (nickase) may be Nb.BsrDI, Nt.BspQI or a combination thereof.

In some embodiments that utilise a nickase enzyme, the nickase enzyme may be removed from the assay or inactivated following cleavage of the DNA minicircle to prevent unwanted cleavage of the RCA products.

As noted above, in some embodiments, the parental minicircle plasmid from which the DNA minicircle is produced may be arranged such that the second domain of the plasmid, i.e. the plasmid backbone, comprises one or more nickase recognition sequences on each strand. Accordingly, in such embodiments, the addition of the nickase to the reaction mixture not only results in the cleavage of a single strand of the DNA minicircle in order to generate a primer for the RCA reaction, but also results in the cleavage of the backbone of the parental minicircle plasmid which remains following the recombination reaction, and of any un-recombined (intact) parental minicircle plasmid which may be present in the mixture, thus preventing unwanted contamination and reducing the need for subsequent additional purification steps. In some embodiments, the parental minicircle plasmid may comprise a plurality of nickase recognition sites on each strand.

The cleavage enzyme that cleaves a single strand of the DNA minicircle (e.g. nickase) may cleave at any site within the DNA minicircle. In some embodiments, the DNA minicircle may comprise a specific domain (a single strand cleavage site or domain, e.g. a nickase site) at which the cleavage enzyme may act. In a representative embodiment, the DNA minicircle may comprise a sequence between the cleavage domains or between a cleavage domain and the junction sequence, which is not the polynucleotide sequence and which may function as the single strand cleavage site or domain. The fact that the sequence recognised by the cleavage enzyme (the single strand cleavage site or domain) is not in the polynucleotide sequence ensures that the polynucleotide produced from the 5′ end of the RCA product is not truncated. The single strand cleavage site or domain may be designed to be of a different length to the polynucleotide sequence, akin to the cleavage domains and RCA primer binding site discussed above, to ensure that it can be readily separated from the single stranded DNA polynucleotides upon cleavage of the RCA product. Thus, in some embodiments, the RCA primer binding site also functions as the single strand cleavage site or domain or vice versa.

Any DNA polymerase with at least some strand displacement activity may be used in the RCA reaction of the invention. Strand displacement activity ensures that once the polymerase has extended around the DNA minicircle, it can displace the primer sequence and the elongating product and continue to “roll” around the template. In embodiments where the nicked strand of the DNA minicircle provides the primer for RCA extension, the strand displacement activity ensures that the polymerase can displace the nicked strand. Suitable DNA polymerase enzymes with at least some strand displacement activity include phi29 DNA polymerase, E. coli DNA polymerase I, Bsu DNA polymerase (large fragment), Bst DNA polymerase (large fragment) and Klenow fragment. As used herein, the term “DNA polymerase” includes not only naturally occurring enzymes but also all such modified derivatives, including also derivatives of naturally occurring DNA polymerase enzymes. For instance, in some embodiments, the DNA polymerase may have been modified to remove 5′-3′ exonuclease activity.

Particularly preferred DNA polymerase enzymes for use in the invention include phi29 DNA polymerase, Bst DNA polymerase and derivatives, e.g. sequence-modified derivatives, or mutants thereof.

Sequence-modified derivatives or mutants of DNA polymerase enzymes include mutants that retain at least some of the functional activity, e.g. DNA polymerase activity and at least some strand displacement activity, of the wild-type sequence. Mutations may affect the activity profile of the enzymes, e.g. enhance or reduce the rate of polymerisation, under different reaction conditions, e.g. temperature, template concentration, primer concentration etc. Mutations or sequence-modifications may also affect the exonuclease activity and/or thermostability of the enzyme.

As noted above, the RCA reaction of the present method may be conducted using conventional nucleotides, functionalised nucleotides, or a mixture of conventional and functionalised nucleotides.

The terms “functionalised single stranded DNA polynucleotide”, “single stranded functionalised DNA polynucleotide” and “functionalised DNA polynucleotide” are used interchangeably herein and refer to a single stranded DNA polynucleotide containing at least one functionalised nucleotide. Thus, a functionalised DNA polynucleotide has additional or alternative properties or characteristics relative to the corresponding polynucleotide containing only conventional nucleotides. For instance, the incorporation of one or more functionalised nucleotides may render the polynucleotide detectable, e.g. by the incorporation of a label, or capable of interacting and/or reacting with another component, i.e. a component with which the corresponding polynucleotide containing only conventional nucleotides does not interact or react. In some embodiments, the modification may render the polynucleotide resistant to degradation, e.g. chemical and/or enzymatic degradation (e.g. nuclease degradation), or may alter the metabolism of the polynucleotide. In some embodiments, the modification may improve the stability of the oligonucleotide, e.g. improve the stability of duplexes formed by the oligonucleotide, such as the thermostability of the duplexes (e.g. melting temperature). In some embodiments, the incorporation of one or more functionalised nucleotides may render the polynucleotide capable of forming secondary or tertiary structures that are not formed by a corresponding polynucleotide containing only conventional nucleotides.

Accordingly, it will be understood that in the context of a functionalised single stranded oligonucleotide, the term “single stranded” refers to an oligonucleotide which is single stranded under denaturing conditions, e.g. following the application of heat or suitable chemical denaturing agents, i.e. an oligonucleotide with only one continuous backbone (one strand). As noted above, this does not preclude a functionalised single stranded oligonucleotide from forming secondary or tertiary structures. For example, the functionalised single stranded oligonucleotide may comprise regions of self-complementarity, and thus may be capable of forming a hairpin or stem-loop structure, mediated by one region of the functionalised single stranded oligonucleotide hybridising to a complementary region elsewhere in the same oligonucleotide.

Functionalisation of a conventional nucleotide may be achieved by alteration of, or modification to, the structure of any part of the nucleotide. Thus, a functionalised nucleotide may contain a modification, e.g. a chemical modification, on the nucleobase, sugar or group involved in the internucleotide linkage. In some preferred embodiments, the functionalised nucleotide contains a modification, e.g. a chemical modification, on the nucleobase.

Modifications at various positions are described in more detail below and it is contemplated that any of the specific positions described below may be modified with any of functional groups described below.

For instance, the substitution of the C5 position in pyrimidines (i.e. cytosine, thymine and uracil) with a group that is small, rigid and hydrophobic may improve base stacking interactions of, and/or stabilize duplexes formed by, oligonucleotides comprising functionalised nucleotides containing the substituted pyrimidines. A small, rigid and hydrophobic group may be an alkynyl group, e.g. methyl, ethynyl, propynyl, or a halogen group, e.g. fluoro, chloro or bromo. Thus, in some embodiments, the functionalised nucleotide comprises a pyrimidine with a substitution at the C5 position, e.g. an alkynyl group or halogen as defined herein.

In some embodiments, a modification that may render the nucleotide detectable may involve the incorporation of a label into the nucleotide. Any label may find utility in the invention and may be a directly or indirectly signal giving molecule. For instance, directly signal giving labels may be fluorescent molecules, i.e. the functionalised nucleotides may be fluorescently labelled nucleotides. Indirectly signal giving labels may be, for example, biotin molecules, i.e. the labelled nucleotides may be biotin labelled nucleotides, which require additional steps to provide a signal, e.g. the addition of streptavidin conjugated to an enzyme which may act on a chemical substrate to provide a detectable signal, e.g. a visibly detectable colour change. In some embodiments, the label is incorporated in (conjugated to) the nucleobase.

Thus, in some embodiments, the functionalised nucleotide comprises a biotin group conjugated to a nucleobase. In some embodiments, the biotin group may be conjugated to the nucleobase indirectly, e.g. via a linker or linking domain and a suitable linker may be readily selected from those well-known in the art. For instance, the linker may be selected to facilitate the interaction between biotin and streptavidin, i.e. to minimise or prevent steric hindrance. In some embodiments, the biotin group is conjugated to a pyrimidine at the C5 position. In a representative embodiment, the biotin containing functionalised nucleotide may be Biotin-16-Aminoallyl-2′-dUTP, Biotin-16-Aminoallyl-2′-dTTP or Biotin-16-Aminoallyl-2′-dCTP.

In some embodiments, the nucleotide may be labelled with a sterol group, i.e. the nucleotide may comprise a sterol group. In some embodiments, the nucleotide may be labelled with or may comprise a cholesterol group.

A directly detectable label is one that can be directly detected without the use of additional reagents, while an indirectly detectable label is one that is detectable by employing one or more additional reagents, e.g. where the label is a member of a signal producing system made up of two or more components. In many embodiments, the label is a directly detectable label, where directly detectable labels of interest include, but are not limited to: fluorescent labels, coloured labels, radioisotopic labels, chemiluminescent labels, and the like. Any spectrophotometrically or optically-detectable label may be used. In other embodiments the label may provide a signal indirectly, i.e. it may require the addition of further components to generate signal. For instance, the label may be capable of binding a molecule that is conjugated to a signal giving molecule.

In some embodiments, the functionalised nucleotide is a fluorescently labelled nucleotide. Whilst fluorescent labels require excitation to provide a detectable signal, as the source of excitation is derived from the instrument/apparatus used to detect the signal, fluorescent labels may be viewed as directly signal giving labels.

Fluorescent molecules that may be used to label nucleotides are well-known in the art. Fluorophores have been identified with excitation and emission spectra ranging from UV to near IR wavelengths. Thus, the fluorophore may have an excitation and/or emission wavelength in the UV, visible or IR spectral range.

The fluorophore may be a protein, peptide, small organic compound, synthetic oligomer or synthetic polymer. In some embodiments, the fluorophore is a small organic compound, e.g. an organic compound with a molecular weight of 5000 Da or less. Thus, in some embodiments, the fluorophore has a molecular weight of 4000 Da or less, such as 3500 Da, 3000 Da, 2500 Da, 2250 Da, 2000 Da, 1900 Da, 1800 Da, 1700 Da, 1600 Da, 1500 Da or less.

Thus, the fluorophore may be a xanthene derivative (e.g. fluorescein, rhodamine, Oregon green, eosin, Texas red), a cyanine derivative (e.g. cyanine, indocarbocyanine, oxacarbocyanine, thiacarbocyanine, merocyanine), a squaraine derivative (e.g. ring-substituted squaraine, Seta, SeTau), a naphthalene derivative (e.g. dansyl or prodan derivative), a coumarin derivative, an oxadiazole derivative (such as pyridyloxazole, nitrobenzoxadiazole and benzoxadiazole), an anthracene derivative (such as an anthraquinone, including DRAQ5, DRAQ7 and CyTRAK Orange), a pyrene derivative (e.g. cascade blue), an oxazine derivative (e.g. Nile red, Nile blue, cresyl violet, oxazine 170), an acridine derivative (such as proflavin, acridine orange, acridine yellow), an arylmethine derivative (e.g. auramine, crystal violet, malachite green) or a tetrapyrrole derivative (such as porphyrin, phthalocyanine, bilirubin).

Specific examples of fluorophores or fluorophore series that may find utility in the present invention include Alexa Fluors (such as Alexa Fluor 488, Alexa Fluor 647 etc.), Atto, Cyanine (Cy), indocyanine, sulfocyanine, DyLight, Abberior STAR, Chromeo, Oregon Green, Fluorescein, Texas red, Rhodamine, Silicon-rhodamine (SiR), Squaraine, FluoProbes, Tetrapyrrole, Bodipy, HiLyte, Quasar, CAL fluor, Coumarin, Seta, CF, Tracy, IRDye, CruzFluor, Tide Fluor, Oyster, iFluor, Chromis and Brilliant Violet and fluorescent derivatives or analogues thereof.

In some embodiments, the functionalised nucleotide contains a cyanine fluorescent label, such as Cy3. In some particular embodiments, the functionalised nucleotide is dATP labelled with Cy3, e.g. 7-Propargylamino-7-deaza-ATP-Cy3.

In some embodiments, the functionalised nucleotide contains an atto fluorescent label, such as atto-488. In some particular embodiments, the functionalised nucleotide is dATP labelled with atto-488, e.g. 7-Propargylamino-7-deaza-ATP-Atto-488.

In some embodiments, a modification that may render the nucleotide reactive involves the incorporation of a reactive group, e.g. a group capable of forming a covalent bond with another chemical group, e.g. a chemical group on a molecule or component to be conjugated to the functionalised DNA polynucleotide, such as a label as defined herein. Potential reactive groups include nucleophilic functional groups (alkynes, alkenyls, amines, alcohols, thiols, hydrazides, azides), electrophilic functional groups (aldehydes, esters, vinyl ketones, epoxides, isocyanates, maleimides), functional groups capable of cycloaddition reactions, forming disulfide bonds, or binding to metals. Specific examples include ethyne (acetylene), propyne, 1-butyne, 2-butyneazide, vinyl (ethenyl), propenyl, 1-butenyl, primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, azides and maleimides.

In some embodiments, a modification that may render the nucleotide reactive involves the incorporation of a reactive group that is capable of reacting with another chemical group, e.g. a chemical group on a molecule or component to be conjugated to the functionalised DNA polynucleotide, via click chemistry. As used herein, the term “click chemistry,” generally refers to reactions that are modular, wide in scope, give high yields, generate only inoffensive by-products, such as those that can be removed by nonchromatographic methods, and are stereospecific (but not necessarily enantioselective). See, e.g., Angew. Chem. Int. Ed., 2001, 40(11):2004-2021, which is entirely incorporated herein by reference. In some cases, click chemistry can describe pairs of functional groups that can selectively react with each other in mild, aqueous conditions. Accordingly, click chemistry groups are suitable for the conjugation of additional functional groups to the polynucleotides of the present method. Common click chemistry reactions include azide-alkyne cycloadditions, alkyne-nitrone cycloadditions, alkene-tetrazine reactions and alkene-tetrazole reactions. Accordingly, the functionalised nucleotide may comprise an azide group, an alkyne group, an alkene group, a nitrone group, a tetrazine group or a tetrazole group.

A specific example of click chemistry reaction can be the Huisgen 1,3-dipolar cycloaddition of an azide and an alkyne, i.e., Copper-catalysed reaction of an azide with an alkyne to form a 5-membered heteroatom ring called 1,2,3-triazole. The reaction can also be known as a Cu(I)-Catalyzed Azide-Alkyne Cycloaddition (CuAAC), a Cu(I) click chemistry or a Cu+ click chemistry. Catalyst for the click chemistry can be Cu(I) salts, or Cu(I) salts made in situ by reducing Cu(II) reagent to Cu(I) reagent with a reducing reagent (Pharm Res. 2008, 25(10): 2216-2230). Known Cu(II) reagents for the click chemistry can include, but are not limited to, Cu(II) (TBTA) complex and Cu(II) (THPTA) complex. TBTA, which is tris-[(1-benzyl-1H-1,2,3-triazol-4-yl)methyl]amine, also known as tris-(benzyltriazolylmethyl)amine, can be a stabilizing ligand for Cu(I) salts. THPTA, which is tris-(hydroxypropyltriazolylmethyl)amine, can be another example of stabilizing agent for Cu(I). Other conditions can also be accomplished to construct the 1,2,3-triazole ring from an azide and an alkyne (e.g. a cycloalkyne, such as cyclooctyne or cyclononyne) using copper-free click chemistry, such as by the Strain-promoted Azide-Alkyne Click chemistry reaction (SPAAC, see, e.g., Chem. Commun., 2011, 47:6257-6259 and Nature, 2015, 519(7544):486-90), each of which is entirely incorporated herein by reference.

In some embodiments, the functionalised nucleotide contains a reactive group in the in the sugar group, e.g. a modification at position 2 in the deoxyribose sugar, such as substituting the hydrogen with a fluoro, chloro, bromo or azide group. In some embodiments, the functionalised nucleotide is a 2′-Azido-dNTP, e.g. 2′-Azido-dATP.

In some embodiments, the functionalised nucleotide contains a reactive group in the nucleobase, particularly selected from an alkyne, alkenyl, thio or halogen group. As noted above, the functionalised nucleotide may comprise a pyrimidine comprising a substitution at the C5 position. In some embodiments, the alkyne group is ethyne, e.g. the functionalised nucleotide is an ethynyl-dNTP, such as 5′-Ethynyl-dUTP. Alternatively, the alkyne group may be a propynyl group, e.g. the functionalised nucleotide is a propynyl dNTP, such as 5′-Propynyl-dUTP. In some embodiments, the alkenyl group is vinyl (ethenyl), e.g. the functionalised nucleotide is a vinyl-dNTP, such as 5′-vinyl-dUTP. In some embodiments, the functionalised nucleotide is a thio-dNTP, such as 4′-thio-dTTP. In some embodiments, the halogen group is bromine, e.g. the functionalised nucleotide is a bromo-dNTP, such as 5′-Bromo-dUTP. The incorporation of halogen groups into functionalised oligonucleotides can be used to promote nucleophilic aromatic substitutions or UV mediated crosslinking, e.g. with proteins. Thus, in some embodiments, functionalised oligonucleotides produced by the present method may be further modified to comprise aromatic groups or may by conjugated to other molecules, e.g. proteins or peptides, via UV mediated crosslinking.

In some embodiments, a functionalised nucleotide may contain a group capable of interacting with another component, e.g. interacting via a non-covalent bond. For instance, the nucleotide may be modified to incorporate one part or component of a cognate binding pair, e.g. an affinity binding partner, e.g. biotin or a hapten, capable of binding to its binding partner, i.e. a cognate binding partner, e.g. streptavidin or an antibody. Such functionalised nucleotides may, for example, find utility in the production of functionalised DNA polynucleotides that may be immobilised on a solid support.

In some embodiments, the functionalised nucleotide contains a modification that renders the polynucleotide containing the nucleotide resistant to degradation, e.g. chemical and/or enzymatic degradation (e.g. nuclease degradation). In some embodiments, the nucleotide contains a modification in the sugar group, e.g. a modification at position 2 in the deoxyribose sugar, such as substituting the hydrogen with a fluoro, chloro, O-methyl or O-ethyl group. Thus, in some embodiments, the functionalised nucleotide is a 2′-fluoro-dNTP, e.g. 2-fluoro-UTP. In some embodiments, the functionalised nucleotide comprises an O-Me group, e.g. 2′-O-Methyl-ATP. In some embodiments, the nucleotide contains a modification in the phosphate group that forms the internucleotide linkage, such as substituting an oxygen with a sulfur, e.g. the nucleotide contains a phosphorothionate group. Thus, in some embodiments, the functionalised nucleotide is nucleotide thiotriphosphate, e.g. 2-deoxythymidine-5′-O-(1-thiotriphosphate), 2-deoxycytidine-5′-O-(1-thiotriphosphate), 2-deoxyuridine-5′-O-(1-thiotriphosphate), 2-deoxyadenosine-5′-O-(1-thiotriphosphate) or 2-deoxyguanosine-5′-O-(1-thiotriphosphate). In some embodiments, the functionalised nucleotides contain a modification in the nucleobase, such as an amino, methyl, ethyl or propynyl modification, e.g. 2-amino-dATP, 5-methyl-dCTP, C-5 propynyl-dCTP or C-5 propynyl-dUTP.

In some embodiments, the functionalised nucleotide contains a modification that affects the thermostability of the oligonucleotide. In some embodiments, the nucleotide comprises a locked ribose sugar, i.e. comprises an additional covalent bond between the 2′ oxygen and the 4′ carbon of the pentose ring. In some embodiments, the nucleotide is an LNA (locked nucleic acid) nucleotide, i.e. LNA-NTP such as LNA-ATP. The locked ribose conformation enhances base stacking and thus increases the melting temperature of oligonucleotides comprising LNA nucleotides.

Thus, in some embodiments, the functionalised nucleotides that may be used in the invention include: nucleotides comprising an (internalized) alkyne or azide group, fluorescently labelled nucleotides, nucleotides comprising a sterol group, nucleotides comprising a polyether group, nucleotides comprising a metal complex, nucleotides comprising a vinyl group, nucleotides comprising a thiol group, thionated nucleotides, nucleotides modified to have increased nuclease resistance, nucleotides comprising a chemical group capable of participating in click chemistry, nucleotides that affect (e.g. increase) the thermostability of the oligonucleotide (e.g. LNA-nucleotides) or a combination thereof. The incorporation of these functionalised nucleotides into the single stranded polynucleotides of the present invention can provide a range of useful functions. For example, single stranded polynucleotides comprising fluorophores can be used as sequence specific fluorescent probes. The inclusion of thiolated nucleotides in a single stranded polynucleotide allows the polynucleotide to be labelled with thiol-reactive molecules, and to be used as a probe for molecular detection of such thiol-reactive molecules.

In some embodiments, the functionalised nucleotides that may be used in the invention do not include nucleotides comprising a digoxigenin group. Alternatively put, in some embodiments, the functionalised nucleotides are not digoxigenin labelled nucleotides. In particular, in some embodiments the functionalised nucleotides are not digoxigenin-11-dUTP.

In a preferred embodiment, the functionalised dNTPs are nucleotides comprising an alkyne group or a vinyl group, e.g. a modified nucleobase containing an alkyne (e.g. ethynyl) group or vinyl group, e.g. a nucleotide comprising a pyrimidine with an alkyne or vinyl group at position C5. In another preferred embodiment, the functionalised dNTPs are nucleotides comprising an azide group, e.g. comprising a modified sugar containing an azide group, e.g. a nucleotide comprising an azide group at position 2 of the deoxyribose sugar.

The reaction mixture for the RCA reaction must contain a combination of components capable of generating a RCA product from the template DNA minicircle. For instance, the nucleotides present in the reaction mixture (e.g. the mixture of conventional and functionalised nucleotides) must be capable of hybridising to their respective nucleotides in the DNA minicircle (RCA template) to permit rolling circle amplification. The relative amounts of functionalised and conventional nucleotides present in the reaction mixture may vary depending on the identity of the functionalised nucleotide and the polymerase. Moreover, the relative amount of each nucleotide present in the reaction mixture may be used to control the incorporation of functionalised nucleotides into the RCA product. For instance, increasing the concentration of the functionalised nucleotides (or decreasing the proportion of conventional nucleotides) may result in a greater proportion of functionalised nucleotides in the RCA product (e.g. when the functionalised nucleotide is used in combination with its equivalent conventional nucleotide). Conversely, decreasing the concentration of the functionalised nucleotides (or increasing the proportion of conventional nucleotides) may result in a lower proportion of functionalised nucleotides in the RCA product.

Thus, the functionalised nucleotides may be used in addition to, or entirely in place of, the conventional nucleotides which hybridise to the same DNA base (nucleotide) in the template DNA. In some embodiments, the reaction mixture may contain only one type of functionalised nucleotide. In some embodiments, a combination of different functionalised nucleotides may be used in the same reaction. In some embodiments, all of the functionalised nucleotides in the reaction mixture contain the same type of functional group, e.g. alkyne group. In some embodiments, the functionalised nucleotides in the reaction mixture contain the different types of functional group. By way of example, the different types of functionalised nucleotides may be capable of hybridising to the same DNA base (nucleotide), such as a dATP nucleotide functionalised with a fluorophore and a second dATP nucleotide functionalised with a sterol group. In another representative example, the different types of functionalised nucleotides may be capable of hybridising to different DNA bases (nucleotides), such as a dATP nucleotide functionalised with a fluorophore and a dTTP nucleotide functionalised with a sterol group (or a different fluorophore to the fluorophore on the dATP nucleotide). Thus, any combination of functionalised and conventional nucleotides may be used in the invention. In a preferred embodiment, the RCA reaction mixture contains only one type of functionalised nucleotide.

The amount of functionalised nucleotides present in the reaction mixture can be measured as a relative percentage of the total nucleotides capable of hybridising to a particular DNA base (nucleotide). Alternatively, this value can be considered as the percentage to which the functionalised nucleotide has replaced the corresponding conventional nucleotide. For example, using equal amounts of conventional dATP and a dATP modified with a fluorophore could be expressed as 50% of the total dATP nucleotides being modified (functionalised), or 50% replacement of the conventional dATP nucleotides with functionalised dATP nucleotides.

The relative amount of functionalised nucleotides in the RCA reaction mixture can be varied in order to control the frequency of functionalised nucleotides in the final single stranded polynucleotides. In some embodiments, the functionalised nucleotides may represent up to about 5% of the total nucleotides capable of hybridising to a particular DNA base (nucleotide), for example about 1%, 2%, 3%, 4% or 5%. Alternatively, in some embodiments the functionalised nucleotide may represent a higher proportion, such as 25%, 50%, 75% or 100% of the total nucleotides capable of binding to a particular DNA base (nucleotide).

The relative amount of functionalised nucleotides present may be varied for numerous reasons. For instance, some functionalised nucleotides, such as dATP modified with Cy3 are not available commercially at high concentrations. Moreover, as shown in the Examples, the inventors have determined that the inclusion of functionalised nucleotides may impact the yield of functionalised single stranded DNA polynucleotides produced by the invention, e.g. the use of high relative amounts of the functionalised nucleotide can inhibit the activity of the DNA polymerase responsible for the RCA reaction (e.g. reduce the efficiency at which the RCA product is synthesised), or the cleavage enzyme responsible for cleaving the RCA product and releasing the single stranded functionalised DNA polynucleotides (e.g. reduce the efficiency at which the RCA product is cleaved). The step of hybridising a restriction (cleavage) oligonucleotide to the cleavage domains of the RCA product to form a duplex comprising a restriction endonuclease recognition site may therefore be particularly advantageous in embodiments where functionalised nucleotides are incorporated into the RCA product, particularly the cleavage domains, as these may interfere with the activity (e.g. reduce the efficiency) of the cleavage enzyme.

Notably however, the inventors have surprisingly determined that high yields of functionalised single stranded DNA polynucleotides may be achieved even when using high relative amounts of functionalised nucleotides, e.g. up to about 75%, such as up to about 70%, 65%, 60%, 55% or 50%. In some embodiments, it may be possible to use more than 75% of the functionalised nucleotides, such as about 80%, 85%, 90%, 95% or 100%. In particular, the inventors have unexpectedly found that functionalised nucleotides containing alkyne or vinyl groups in the nucleobase are particularly useful in the invention as they may be incorporated into the RCA product efficiently. Moreover, the RCA product could readily be cleaved to yield the functionalised single stranded DNA polynucleotides, although as discussed below, in some embodiments a higher amount of cleavage enzyme may be required relative to the amount needed to cleavage an equivalent RCA product containing only conventional nucleotides.

Similarly, the inventors have found that functionalised nucleotides containing O-methyl groups in the deoxyribose sugar may completely substitute a conventional nucleotide in the RCA reaction and still yield RCA products. Furthermore, the inventors surprisingly determined that incorporation of these nucleotides into the RCA products does not affect formation of the cleavage domains (e.g. hairpin cleavage domains) or their enzymatic cleavage (e.g. with restriction endonucleases).

Accordingly, the relative amount of functionalised nucleotide that is present in the reaction mixture may be adjusted to optimise the yield of the desired functionalised single stranded polynucleotides or to optimise the generation of the RCA product. Such modifications are within the purview of the skilled person based on the methods described in the Examples below. Thus, in some embodiments, the relative amount of functionalised nucleotides in the reaction mixture may be about 1-5%, 1-10%, 1-25%, 5-25%, 10-25%, 25-50%, 25-75%, 25-100%, 50-75%, 50-100%, or 75-100%.

In addition to the relative amount of functionalised nucleotides in the reaction mixture, the absolute amount of nucleotides (both conventional and functionalised nucleotides) may be adjusted to optimise the yield of the desired functionalised single stranded polynucleotides or to optimise the generation of the RCA product. Such modifications are within the purview of the skilled person based on the methods described in the Examples below.

Following the generation of the RCA product in step (b), the method comprises a step of cleaving the RCA product at the cleavage domains to release the single stranded DNA polynucleotides. As discussed above, the step of cleaving the RCA product may be achieved by contacting the RCA product with a cleavage enzyme under suitable conditions to selectively cleave the RCA product in the cleavage domains.

The term “release” is used in the present context to refer to cleaving the RCA product at the cleavage domains bordering the polynucleotide sequences so as to detach or separate the polynucleotides from the cleavage domains. It is desirable that the release of a given polynucleotide will involve cleavage at both cleavage domains bordering the polynucleotide sequence.

It is not necessary for cleavage to occur at all of the cleavage domains in the RCA product in order to generate the single stranded DNA polynucleotides. The cleavage of a portion of cleavage domains will still result in the release of a portion of single stranded DNA polynucleotides. Thus, in some embodiments, the step of cleaving the RCA product results in cleavage of at least about 30% of the cleavage domains in the RCA product, e.g. at least about 35%, 40%, 45%, 50%, 60%, 70% or 80%. In some embodiments, the step of cleaving the RCA product results in cleavage of at least about 90% of the cleavage domains in the RCA product, e.g. 95% or more.

Alternatively viewed, in some embodiments, the step of cleaving the RCA product results in the release of at least about 30% of the single stranded DNA polynucleotides contained in the RCA product, e.g. at least about 35%, 40%, 45%, 50%, 60%, 70% or 80%. In some embodiments, the step of cleaving the RCA product results in the release of at least about 90% of the single stranded DNA polynucleotides contained in the RCA product, e.g. 95% or more.

Once the single stranded DNA polynucleotides have been released, it may be desirable to isolate, separate or purify the single stranded DNA polynucleotides from the cleavage reaction mixture (e.g. reaction components and/or degradation products such as cleavage domains, uncleaved RCA products etc.) for use in other applications.

Thus, in some embodiments, the method of the present invention further comprises a step of isolating, separating or purifying the single stranded DNA polynucleotides. This isolation, separation or purification may be done by any suitable method known in the art.

In some embodiments, following the isolation, separation or purification step the single stranded DNA polynucleotides are preferably substantially free of any contaminating components derived from the materials or components used in the isolation procedure or in their preparation (e.g. reaction components and/or degradation products such as cleavage domains, uncleaved RCA products etc.). In some embodiments, the single stranded DNA polynucleotides are purified to a degree of purity of more than about 50 or 60%, e.g. more than about 70, 80 or 90%, such as more than about 95 or 99% purity as assessed w/w (dry weight). Such purity levels may include degradation products of the single stranded DNA polynucleotides.

In some embodiments, it may be useful to prepare enriched preparations of the single stranded DNA polynucleotides which have lower purity, e.g. contain less than about 50% of the single stranded polynucleotides of interest, e.g. less than about 40 or 30%.

As discussed above, the invention may result in a mixture (e.g. a library) of single stranded DNA polynucleotides, e.g. where the pseudogene comprises different polynucleotide sequences and/or where the RCA reaction incorporates functionalised nucleotides into the RCA product. Thus, in some embodiments, it may be desirable to further separate the mixture of single stranded DNA polynucleotides, e.g. by size, to obtain specific single stranded DNA polynucleotides (i.e. to isolate specific single stranded DNA polynucleotides) or to generate sub-groups or sub-libraries of single stranded DNA polynucleotides. Any suitable means for separating the mixtures of single stranded polynucleotides to isolate the specific single stranded DNA polynucleotides or sub-groups or sub-libraries of single stranded DNA polynucleotides may be employed.

Thus, in some embodiments, the method comprises a further step of separating single stranded DNA polynucleotides from a mixture of single stranded DNA polynucleotides obtained by the method described above (e.g. a library of single stranded functionalised DNA polynucleotides), to isolate a specific single stranded DNA polynucleotide or a sub-group of single stranded DNA polynucleotides.

For example, the products of the cleavage reaction may be separated by size using gel electrophoresis using an agarose gel or a polyacrylamide gel. The desired polynucleotides can then be isolated from the gel and purified further, if necessary, according to methods known in the art. Other methods for purifying, isolating or separating the polynucleotides of the invention utilise chromatography (e.g. HPLC, size-exclusion, ion-exchange, affinity, hydrophobic interaction, reverse-phase) or capillary electrophoresis.

As mentioned above, the functionalised polynucleotides produced by the method described above may contain a reactive group that is capable of reacting with another chemical group, e.g. a chemical group on a molecule or component to be conjugated to the functionalised polynucleotide, e.g. via click chemistry. For instance, conjugating additional molecules or components (which themselves may comprise or be viewed as functional groups) to the single stranded functionalised DNA polynucleotides may be particularly useful for incorporating large or bulky groups, such as groups that may inhibit or lower the efficiency of the present method if present in the functionalised nucleotides used in the RCA reaction. Thus, for example, functionalised polynucleotides may be produced that contain molecules or components that could not be incorporated by a polymerase directly during the RCA reaction, or would only be incorporated at low efficiencies or yields. Additionally or alternatively, subjecting the functionalised polynucleotides obtained by the method to a further conjugation step may increase the diversity of the structures present in a functionalised polynucleotide library.

Accordingly, in some embodiments, the method further comprises a step of conjugating a molecule or component to the functionalised polynucleotide(s) via a functional (e.g. reactive) group in the polynucleotide, such as via click chemistry. In some preferred embodiments, the molecule or component is conjugated to the functionalised polynucleotide via an alkyne, vinyl or azide group (i.e. an alkyne, vinyl or azide group in a functionalised nucleotide incorporated into the RCA product in the method defined herein).

The term “conjugation” in the context of the present invention with respect to linking or joining a molecule or component to a functionalised polynucleotide (e.g. an alkyne, vinyl or azide group in said functionalised polynucleotide) refers to joining said molecule or component to said polynucleotide via a covalent bond. In particular, this conjugation may occur via a click chemistry reaction.

An example of a specific click chemistry reaction that may be used to conjugate additional molecules or components to single stranded functionalised DNA polynucleotides of the present invention comprising one or more alkyne groups is the azide-alkyne cycloaddition. In order to achieve the desired conjugation, the polynucleotide comprising the alkyne group may be incubated for a suitable period of time with a molecule or component (e.g. a label such as a fluorophore) containing an azide group. The azide-alkyne cycloaddition reaction commonly uses a copper catalyst, particularly a copper(I) catalyst. In some embodiments, the polynucleotide and the azide-containing molecule or component may be incubated in the presence of copper sulfate. A reducing agent may also be used to generate the active copper(I) catalyst. The reducing agent may be, for example, sodium ascorbate.

A further representative example for conjugating additional molecules or components to single stranded functionalised DNA polynucleotides of the present invention comprising one or more vinyl groups may utilise the alkene-tetrazine reaction. This reaction has the advantage of being copper free. Moreover, it is entirely orthogonal to the aforementioned alkyne-azide click chemistry reaction. Accordingly, a single stranded functionalised DNA polynucleotide comprising both an alkyne group and a vinyl group could participate in two click chemistry reactions independently to conjugate two different additional molecules or components to the same polynucleotide.

Thus, in some embodiments, the invention may be seen as providing a two-step method for producing single stranded functionalised DNA polynucleotides comprising a first step of incorporating functionalised nucleotides (e.g. comprising reactive groups, such as groups capable of participating in click chemistry reactions, e.g. alkyne, vinyl or azide groups) into a polynucleotide using the method described herein, and a second step of conjugating additional molecules or components to the single stranded polynucleotides via the functional groups in the functionalised nucleotides.

It will be evident that any desirable molecules or components (i.e. entities) may be conjugated to the functional groups in the single stranded polynucleotides produced by the present method. Such molecules or components simply require the presence of a group (e.g. reactive group) capable of reacting with a functional group in the polynucleotide to form a covalent bond. In some embodiments, the molecule or component may be a nucleic acid molecule, protein, peptide, small-molecule organic compound (e.g. a sterol, such as cholesterol), fluorophore, metal-ligand complex, polysaccharide, nanoparticle, nanotube, polymer, cell, organelle, vesicle, virus, virus-like particle or any combination of these.

The cell may be a prokaryotic or eukaryotic cell. In some embodiments the cell is a prokaryotic cell, e.g. a bacterial cell.

In some embodiments, the functionalised polynucleotide may be conjugated or fused to a compound or molecule which has a therapeutic or prophylactic effect, e.g. an antibiotic, antiviral, vaccine, antitumour agent, e.g. a radioactive compound or isotope, cytokine, toxin, oligonucleotide, nucleic acid encoding gene or nucleic acid vaccine.

In some embodiments, the functionalised polynucleotide (e.g. an aptamer) may be conjugated or fused to a label, e.g. a radiolabel, a fluorescent label, luminescent label, a chromophore label as well as to substances and enzymes which generate a detectable substrate, e.g. horse radish peroxidase, luciferase or alkaline phosphatase. This detection may be applied in numerous assays where antibodies are conventionally used, including Western blotting/immunoblotting, histochemistry, enzyme-linked immunosorbent assay (ELISA), or flow cytometry (FACS) formats. Labels for magnetic resonance imaging, positron emission tomography probes and boron 10 for neutron capture therapy may also be conjugated to the functionalised polynucleotides described herein.

In some embodiments, the molecule or component may be selected from the group consisting of: a fluorophore, a sterol (e.g. cholesterol), a polyether, a metal complex, a thiol containing molecule, a molecule containing a group providing increased nuclease resistance and a molecule containing a group capable of participating in a click chemistry reaction.

It will be evident that the molecule or component conjugated to the functionalised polynucleotide may interact with other molecules and such interactions may be covalent or non-covalent interactions. For instance, a peptide conjugated to the polynucleotide may interact with its cognate binding partner, such as an antibody, non-covalently. In a further example, a molecule containing a group capable of participating in a click chemistry reaction may be conjugated to another molecule or component as defined above via reaction with a reactive group of said molecule or component to form a covalent complex.

In a further aspect, the present invention provides a kit, particularly a kit for use in producing single stranded DNA polynucleotides, said kit comprising:

(i) a DNA minicircle obtained from a parental minicircle plasmid, wherein the DNA minicircle comprises a polynucleotide sequence bordered by cleavage domains (e.g. a DNA minicircle as defined above); or

(ii) a parental minicircle plasmid as defined above; and

(iii) one or more additional components for use in the method of the invention, optionally wherein the one or more additional components are: (a) one or more cleavage enzymes that cleave the cleavage domains of (i); and/or (b) functionalised dNTPs as defined herein.

In some embodiments, the kit may comprise a DNA polymerase enzyme capable of performing rolling circle amplification, i.e. comprising at least some strand displacement activity, as defined herein. For instance, the kit may comprise a phi29 polymerase or derivative thereof.

In some embodiments, the kit may comprise a recombinase enzyme capable of recombining the parental minicircle plasmid to form a DNA minicircle as defined herein.

In some embodiments, the kit may comprise nucleotides in the form of dNTPs.

In some embodiments, the kit may comprise a host cell as defined above, e.g. for propagating the parental minicircle plasmid and/or producing the DNA minicircle.

The polynucleotide sequence, cleavage domains, DNA minicircle, recombinase attachment sites of the parental minicircle plasmid, host cell, cleavage enzymes and dNTPs of the kit are as described above.

In a further aspect, the present invention provides a single stranded DNA polynucleotide obtained by the method as described herein, e.g. a plurality of single stranded DNA polynucleotides obtained by the method as described herein. In some embodiments, the single stranded DNA polynucleotides obtained by the method as described herein are functionalised single stranded DNA polynucleotides.

In a yet further aspect, the present invention provides a library comprising a plurality of different single stranded DNA polynucleotides (preferably functionalised single stranded DNA polynucleotides), i.e. a mixture of single stranded DNA polynucleotides (preferably functionalised single stranded DNA polynucleotides) obtained by the method as described herein.

The invention will now be described in more detail in the following non-limiting Examples with reference to the following drawings:

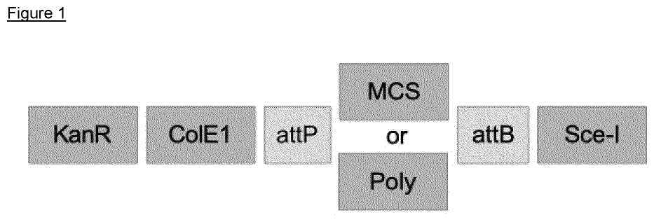

FIG. 1 shows a schematic representation of the parental minicircle plasmid pM1. The multiple cloning site (MCS) comprises a plurality of restriction enzyme cleavage sites to allow the polynucleotide sequence bordered by cleavage domains (poly), i.e. the pseudogene, to be inserted into the plasmid. The pseudogene is located between the two recombination sites attP and attB. Sce-I represents a I-Scel recognition site (cleavage domain). KanR represents a kanamycin resistance gene. ColE1 represents an origin of replication.

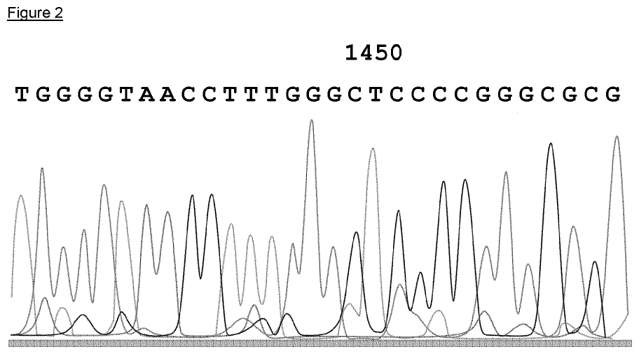

FIG. 2 shows the results of Sanger sequencing of the DNA minicircle obtained from the pM1 plasmid at the precise recombination site following the recombination reaction. The recombination reaction occurs between the attP site and the attB site, and generates the junction sequence 5′-GGGTAACCTTT/GGGCTCCCC-3 (SEQ ID NO: 2).

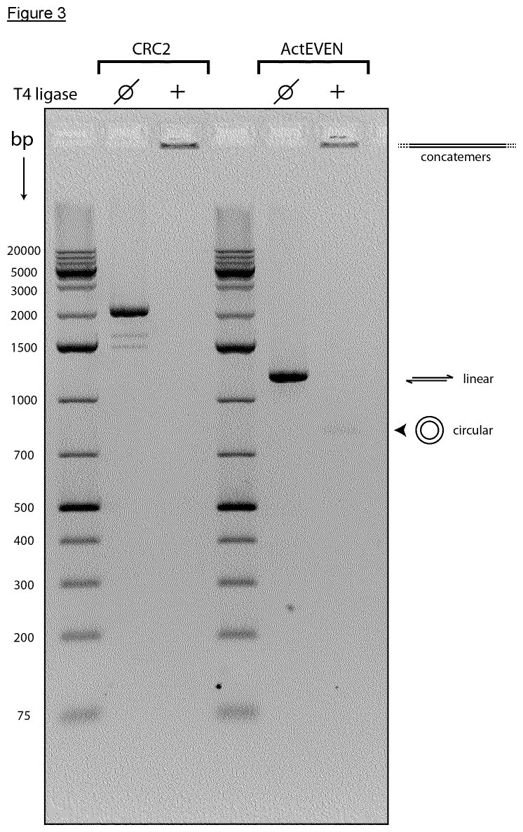

FIG. 3 shows a negative image of a photograph of an agarose gel visualised using UV light following ethidium bromide staining. The agarose gel shows the results of the ligation of linear pseudogenes using T4 ligase (lane 0 is without ligase; lane+is with ligase). The original linear pseudogene is indicated, as are the products of the reaction (either a linear concatemer, resulting from inter-molecular ligation; or a circular DNA molecule, resulting from intra-molecular ligation).

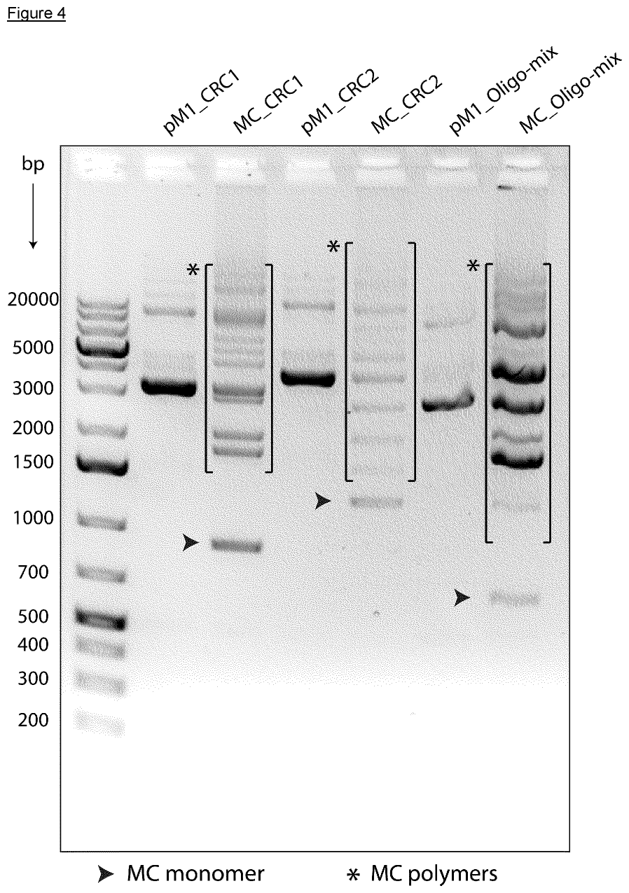

FIG. 4 shows a negative image of a photograph of an agarose gel visualised using UV light following ethidium bromide staining. The agarose gel shows the parental plasmid pM1 containing three different pseudogenes (CRC1, CRC2, and Oligo mix), and the corresponding minicircle (MC) recombination products. Each recombination product comprises a mixture of minicircles including a minicircle monomer (pseudogene plus junction sequence) and multiple minicircle polymers (multiple pseudogenes plus junction sequences in a circular form).

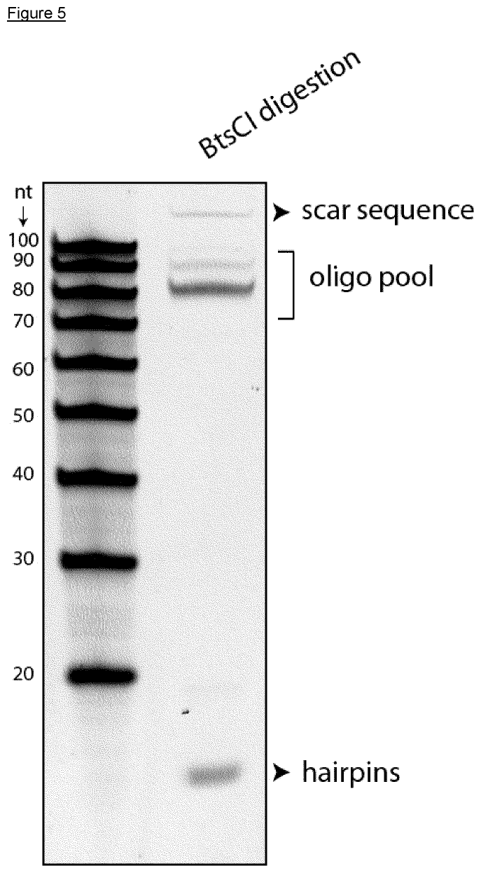

FIG. 5 shows a negative image of a photograph of a denaturing polyacrylamide gel electrophoresis (PAGE) gel visualised using Cy2 light following SybrGold staining. The polyacrylamide gel shows the enzymatic production of a mixture of polynucleotides using the method of the present invention. A pseudogene comprising 8 polynucleotide sequences ranging from 81 to 91 nucleotides in length, bordered by cleavage domains was cloned into the parental minicircle plasmid pM1. DNA minicircles comprising the pseudogene sequence were obtained from the harvested bacterial culture following the recombination reaction and used as the template for an RCA reaction. The enzymatic digestion of the product of this RCA reaction released the hairpin sequences (the remnants of the cleavage domains), the desired polynucleotides and a 150-nucleotide long recombination scar sequence. The scar sequence comprised the recombination product of the merged attB/attP sequences (i.e. the junction sequence), in the centre, surrounded on both sides by remnants of the multiple cloning site for the restriction enzymes XbaI, EcoRV, BamHI, HindII, PstI, StuI, and DraII.

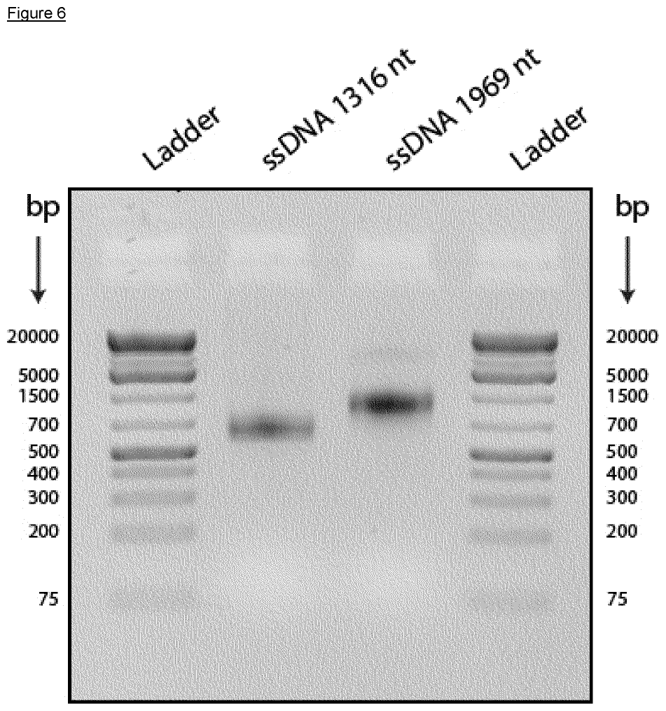

FIG. 6 shows a negative image of a photograph of an agarose gel visualised using UV light following ethidium bromide staining. The agarose gel shows the enzymatic production of two “long” polynucleotides using the method of the present invention. The single stranded DNA polynucleotides obtained had lengths of 1316 nucleotides (A) and 1969 nucleotides (B). Accordingly, they migrated in a 2% agarose gel in a manner consistent with a 700 bp and a 1000 bp double stranded polynucleotide, according to the ladder.

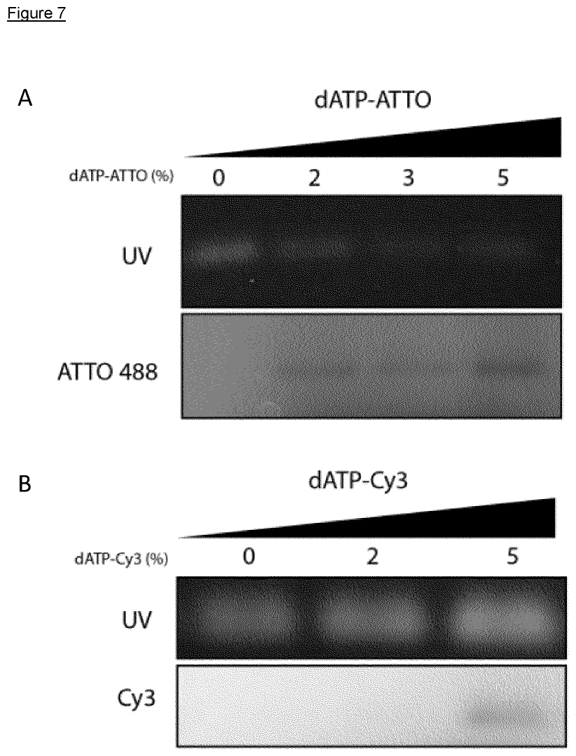

FIG. 7 shows photographs of agarose gels visualised using UV light following ethidium bromide staining (top), and fluorescent imaging using wavelengths corresponding to the emission wavelengths of the fluorophores (bottom). The agarose gels show functionalised single stranded oligonucleotide products of the invention (containing 378 nucleotides) comprising fluorophores ATTO-488 (A) or Cy3 (B).

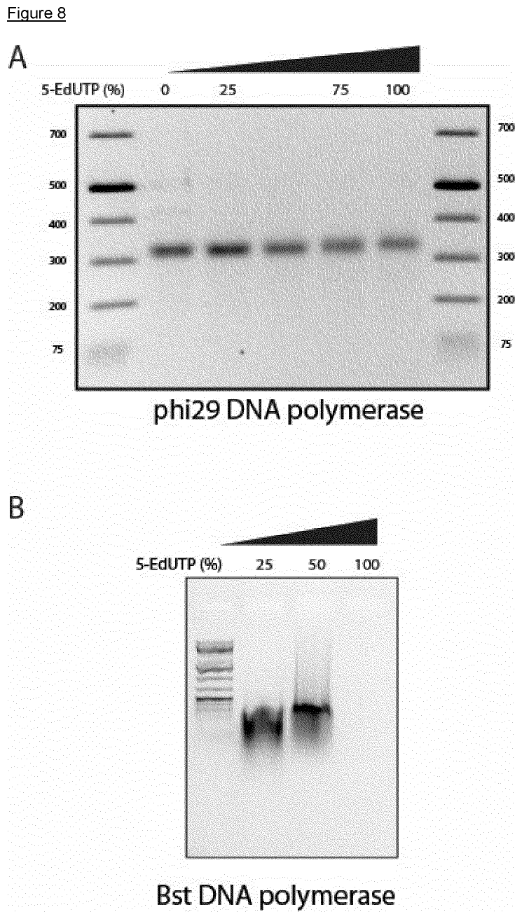

FIG. 8 shows the negative image of a photograph of an agarose gel visualised using UV light following ethidium bromide staining. The agarose gel shows functionalised single stranded oligonucleotide products of the invention (containing 420 nucleotides) comprising 5-ethnyl-dUTP (5-EdUTP) produced using various relative amounts of 5-EdUTP/dTTP nucleotides, i.e. 25%, 50%, 75% and 100% and phi29 DNA polymerase (A) or Bst DNA polymerase (B).

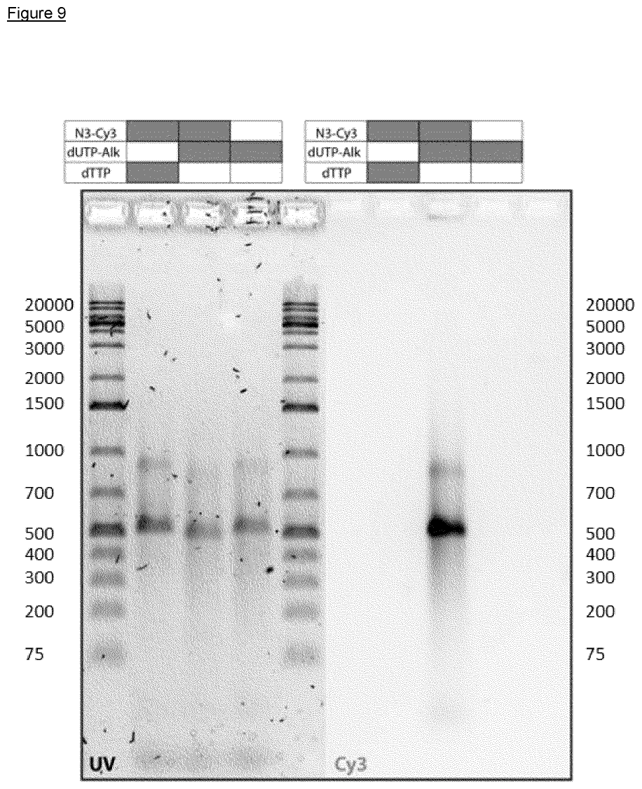

FIG. 9 shows a photograph of an agarose gel visualised using UV light following ethidium bromide staining (left), and fluorescent imaging using wavelengths corresponding to the emission wavelengths of the Cy3 fluorophores (right). The agarose gels show single stranded oligonucleotide products of the invention (containing 420 nucleotides) comprising 5-ethnyl-dUTP (5-EdUTP) or conventional dTTP that were subsequently reacted with Cy3 fluorophore-azide molecule (N3-Cy3). The shaded boxes denote the presence of the indicated species.

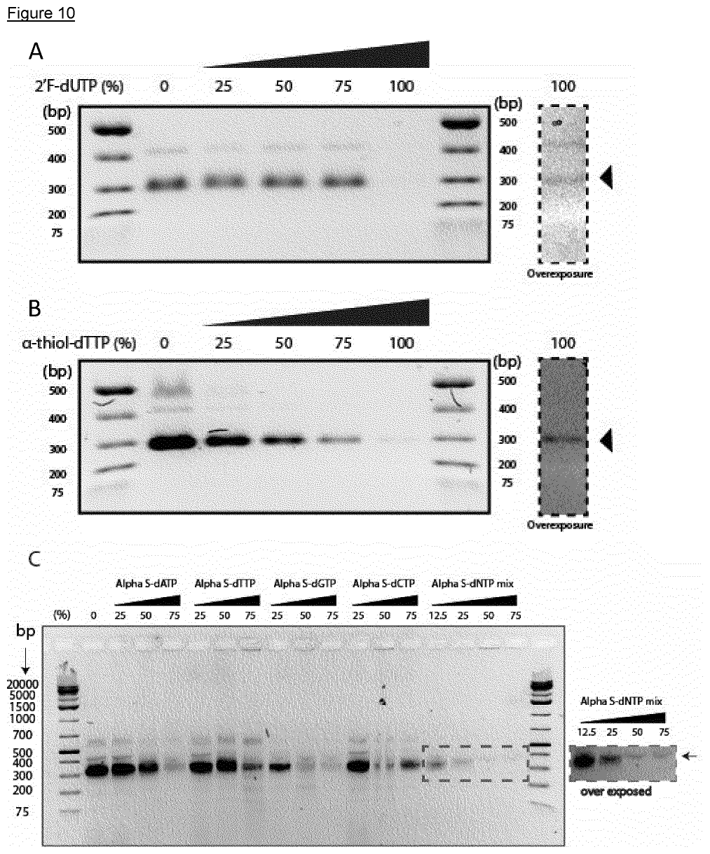

FIG. 10 shows negative images of photographs of agarose gels visualised using UV light following ethidium bromide staining. The agarose gels show functionalised single stranded oligonucleotide products of the invention (containing 420 nucleotides) comprising: (A) 2′-Fluoro-2′-deoxyuridine-5′-triphosphate (2′F-dUTP); (B) 2′-Deoxythymidine-5′-O-(1-Thiotriphosphate) (α-thiol-dTTP); and (C) 2-dNTP Alpha S nucleotides (Alpha S-dATP, Alpha S-dTTP, Alpha S-dCTP, Alpha S-dTTP, and an Alpha S-dNTP mixture), produced using various relative amounts of the functionalised. The right panels in A and B show the lane corresponding to 100% functionalised nucleotides overexposed to show a band corresponding to the RCA product. The right panel in C show the lanes corresponding to Alpha S-dNTP mixture overexposed to show bands corresponding to the RCA product.

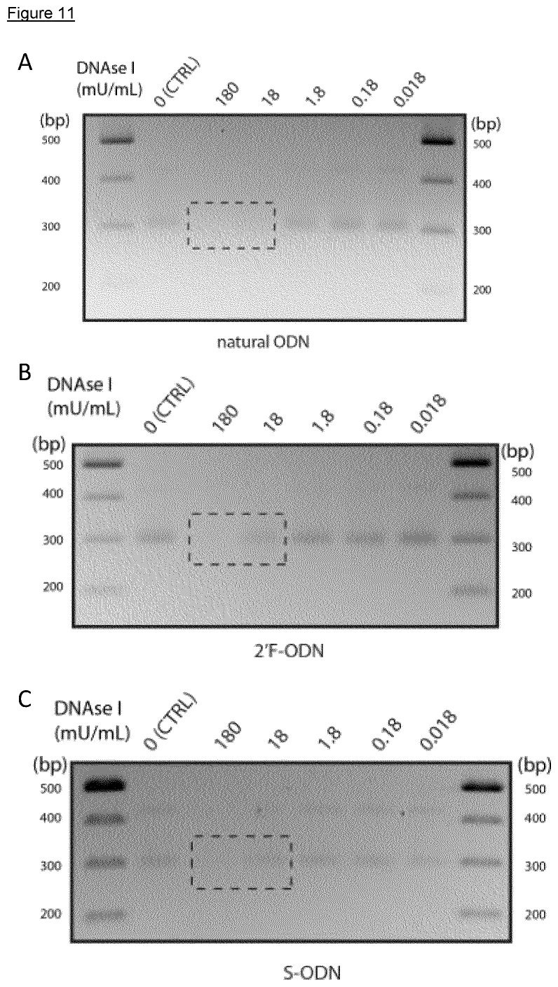

FIG. 11 shows negative images of photographs of agarose gels visualised using UV light following ethidium bromide staining. The agarose gels show oligonucleotides produced by the invention subjected to various concentrations of DNase I, wherein: (A) shows the reaction products of an oligonucleotide containing only conventional nucleotides (natural ODN); (B) shows the reaction products of an oligonucleotide containing 2′-Fluoro-2′-deoxyuridine-5′-triphosphate functionalised nucleotides (2′F-dUTP); and (C) shows the reaction products of an oligonucleotide containing 2′-Deoxythymidine-5′-O-(1-Thiotriphosphate) (S-ODN).

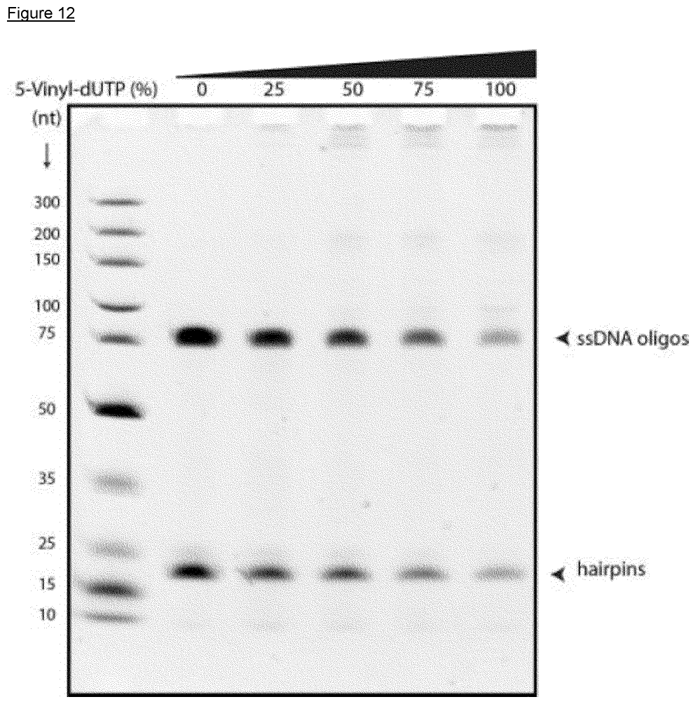

FIG. 12 shows a negative image of a photograph of a denaturing PAGE gel visualised using UV light following SybrGold staining. The PAGE gel shows functionalised single stranded oligonucleotide products of the invention comprising 5-Vinyl-2′-deoxyuridine-5′-triphosphate (5-Vinyl-dUTP) produced using various relative amounts of 5-Vinyl-dUTP nucleotides, i.e. 25%, 50%, 75% and 100%.

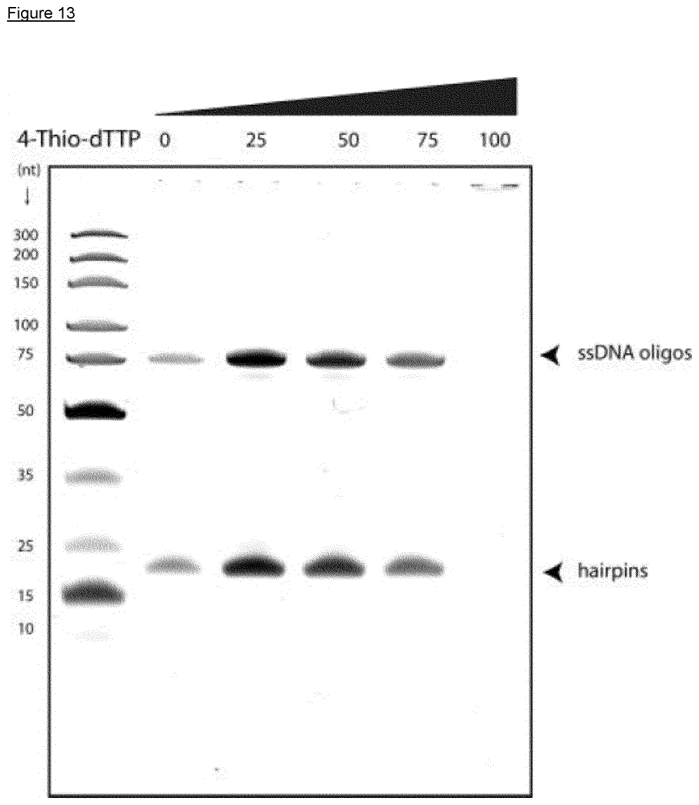

FIG. 13 shows a negative image of a photograph of a denaturing PAGE gel visualised using UV light following SybrGold staining. The PAGE gel shows functionalised single stranded oligonucleotide products of the invention comprising 4-Thiothymidine-5′-Triphosphate (4-Thio-dTTP) produced using various relative amounts of 5-Vinyl-dUTP nucleotides, i.e. 25%, 50%, 75% and 100%.

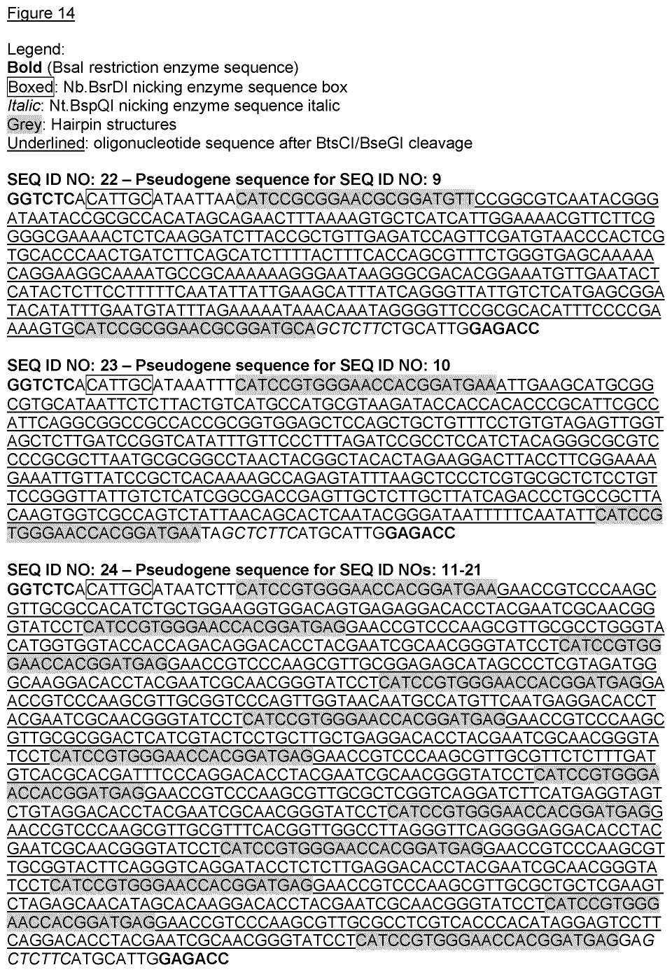

FIG. 14 shows annotated versions of the pseudogene sequences that were used in the production of oligonucleotides having sequences corresponding to SEQ ID NOs: 9-21. The sequences recognised by the cleavage and nicking enzymes, the hairpin sequences, and final oligonucleotide sequences are identified.

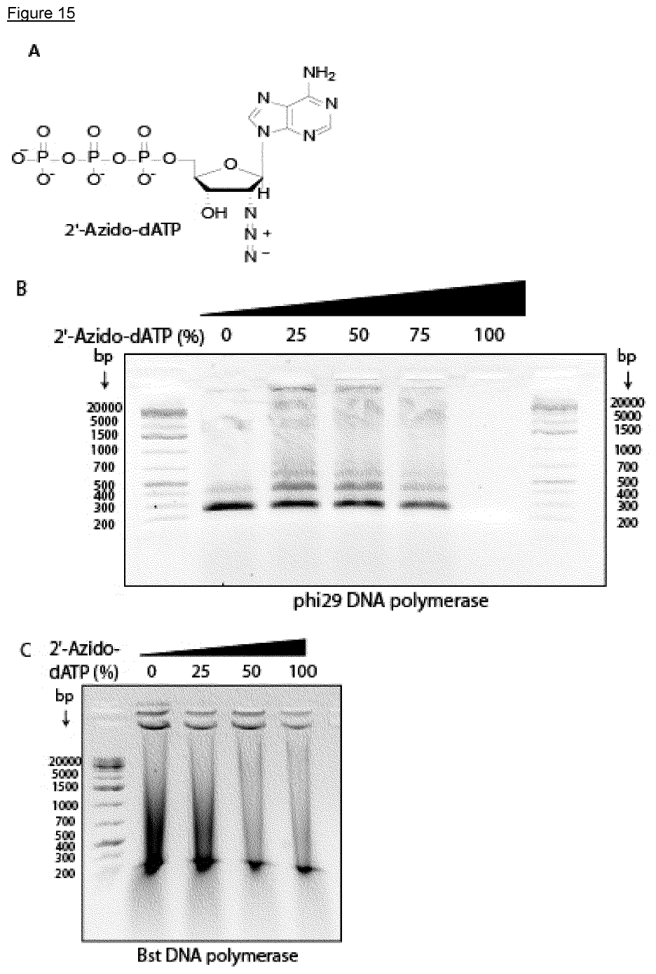

FIG. 15 shows the structure of a 2′-Azido-dATP (A) and negative images of photographs of denaturing PAGE gels visualised using UV light following SybrGold staining (B and C). The PAGE gels show functionalised single stranded oligonucleotide products of the invention comprising 2′-Azido-dATP produced using various relative amounts of 2′-Azido-dATP nucleotides, i.e. 25%, 50%, 75% and 100% and phi29 DNA polymerase (B) or Bst DNA polymerase (C).

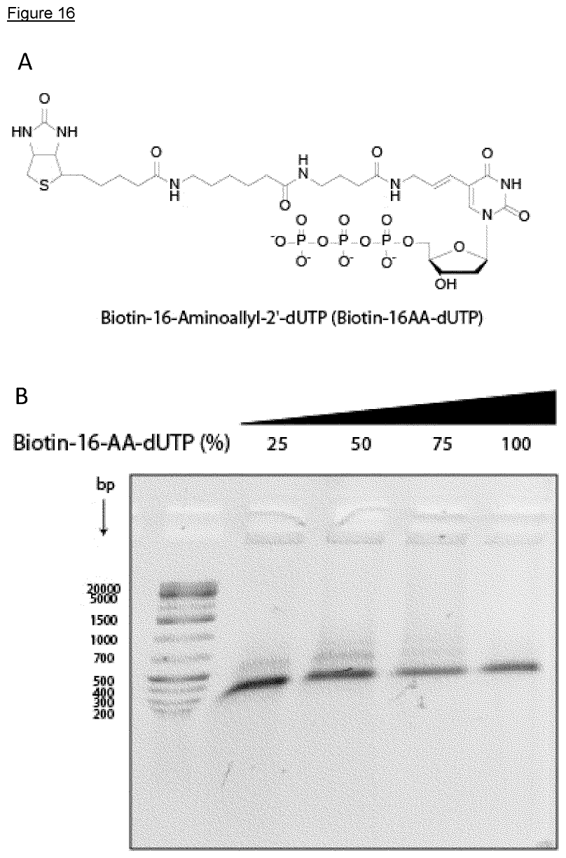

FIG. 16 shows the structure of a Biotin-16-Aminoallyl-2′-dUTP (A) and a negative image of a photograph of a denaturing PAGE gel visualised using UV light following SybrGold staining (B). The PAGE gel shows functionalised single stranded oligonucleotide products of the invention comprising Biotin-16-Aminoallyl-2′-dUTP produced using various relative amounts of Biotin-16-AA-dUTP nucleotides, i.e. 25%, 50%, 75% and 100% and phi29 DNA polymerase.

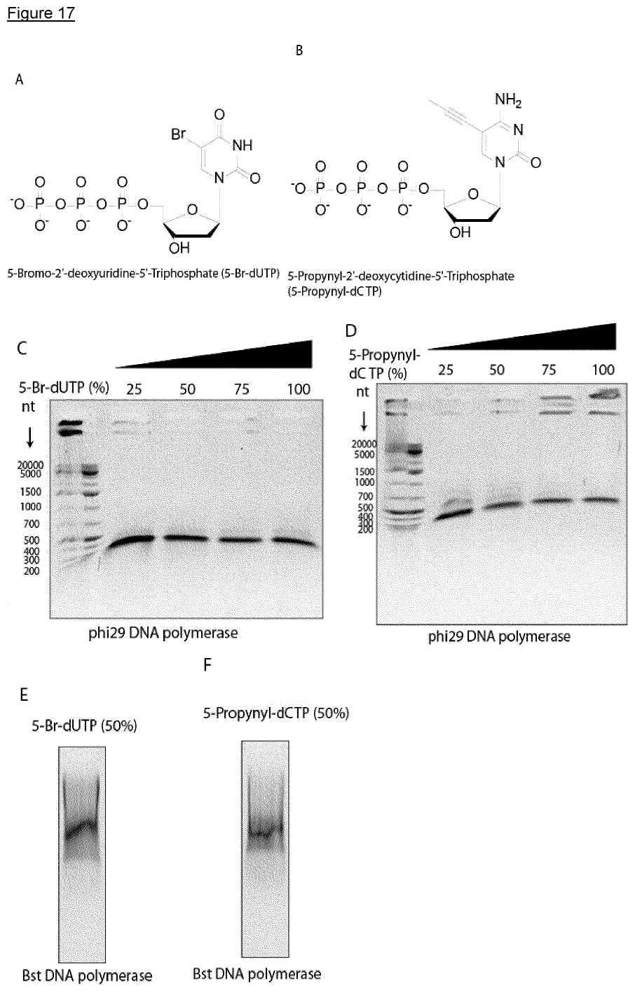

FIG. 17 shows the structures of a 5′-Bromo-2′-deoxyuridine-5′Triphosphate nucleotide (A) and a 5′-Propynyl-2′-deoxycytidine-5′-Triphosphate nucleotide (B); negative images of photographs of denaturing PAGE gels visualised using UV light following SybrGold staining (C, D, E and F). The PAGE gels show functionalised single stranded oligonucleotide products of the invention comprising 5′-Br-dUTP (C and E) or 5′-Propynyl-dCTP (D and F) produced using various relative amounts of the respective functionalised nucleotides, i.e. 25%, 50%, 75% and 100% and phi29 DNA polymerase or Bst DNA polymerase.

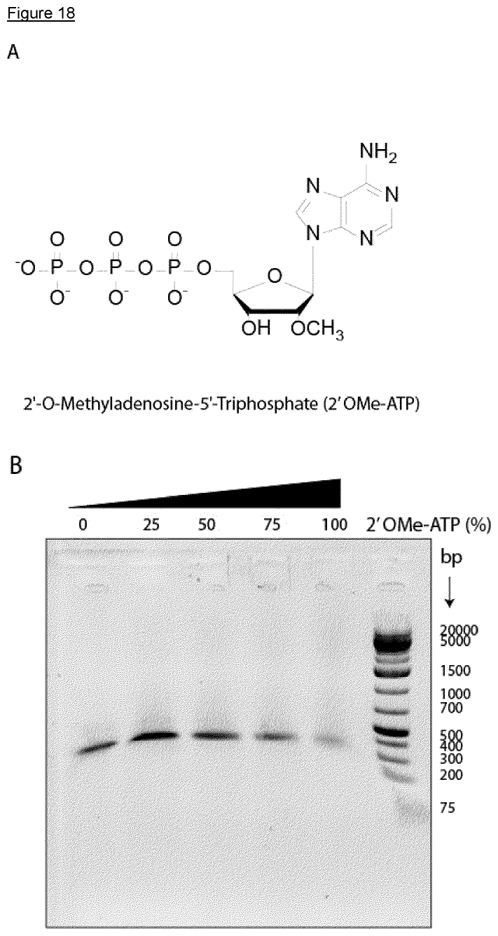

FIG. 18 shows the structure of a 2′-O-Methyladenosine-5′-Triphosphate nucleotide (A) and a negative image of a photograph of a denaturing PAGE gel visualised using UV light following SybrGold staining (B). The PAGE gel shows functionalised single stranded oligonucleotide products of the invention comprising 2′-OMe-ATP produced using various relative amounts of 2′-OMe-ATP nucleotides, i.e. 25%, 50%, 75% and 100% and phi29 DNA polymerase.

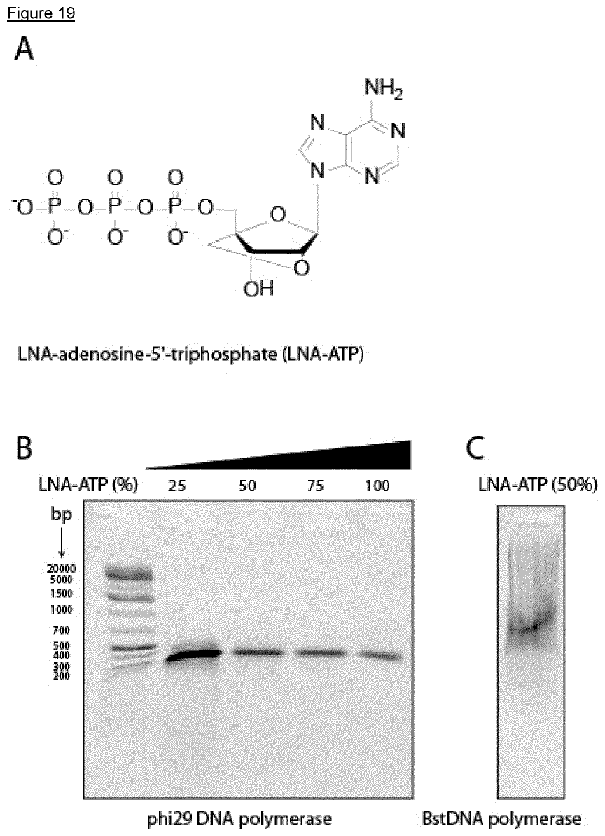

FIG. 19 shows the structure of an LNA-adenosine-5′-triphosphate nucleotide (A) and negative images of photographs of denaturing PAGE gels visualised using UV light following SybrGold staining (B and C). The PAGE gels show functionalised single stranded oligonucleotide products of the invention comprising LNA-ATP produced using various relative amounts of LNA-ATP nucleotides, i.e. 25%, 50%, 75% and 100% and phi29 DNA polymerase (B) or Bst DNA polymerase (C).

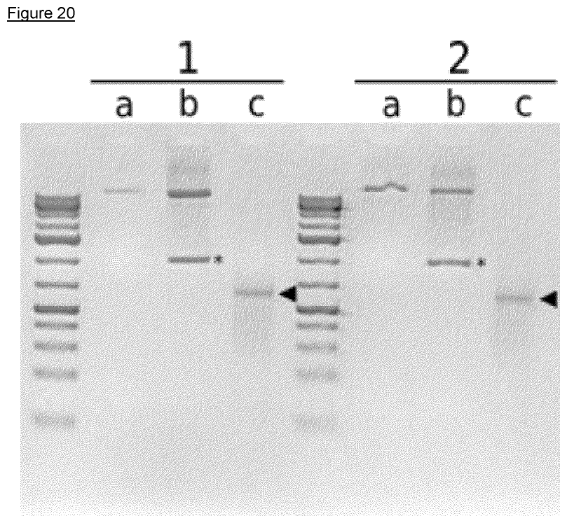

FIG. 20 shows a photograph of a 2% agarose gel wherein: lanes 1a and 2a show the plasmids pM3 (SEQ ID NO: 28) and pM2 (SEQ ID NO: 27), respectively, comprising the CRC1 pseudogene described in the Examples; lanes 1 b and 2b show the recovered recombination products from the pM3 and pM2 plasmids, respectively (minicircle DNA is marked with an “*”); and lanes 1c and 2c show the single stranded polynucleotides generated from the pM3 and pM2 minicircles, respectively.

### EXAMPLES

### Example 1—Efficiency of Circularization of Long Pseudogenes Using T4 Ligase

The MOSIC approach (Ducani et al., Nature Methods 2013) was used to study the circularization efficiency of two long pseudogenes with T4 ligase. The longest pseudogene, here called CRC2 (SEQ ID NO: 8), was designed for the production of a single stranded DNA polynucleotide 1969 bases in length (the total length of the pseudogene after excision was 2041 base pairs). The shorter pseudogene, called ActEVEN (SEQ ID NO: 6), was designed to produce a pool of 11 single stranded oligonucleotides of between 76 and 81 bases in length (the total length of the pseudogene after excision was 1159 base pairs).

The linear pseudogenes (final concentration 5 ng/μl) were mixed with T4 ligase (0.25 U/μl) in 1× rapid ligation buffer at 22° C. for 30 min, followed by an inactivation step at 65° C. for 10 min. As a control, the same reaction mixtures were prepared without T4 ligase. All the reaction mixtures were loaded on a 1.5% agarose gel casted with ethidium bromide (1 μg/ml), run at 150V for 90 minutes and images were acquired by UV trans-illumination (UVITEC). The agarose gel (FIG. 3) showed that the circularization of long linear pseudogenes by T4 ligase is inefficient, and that the ligation reaction favoured the production of concatemers over single circular DNA molecules. It was noted that for the shorter pseudogene, ActEVEN (SEQ ID NO: 6), a faint band corresponding to the single circular DNA molecules was visible, but for the longer DNA pseudogene, CRC2, only concatemers were visible.

### Example 2—Generation of Minicircles from Pseudogenes Cloned into pM1 Plasmids

Three pseudogenes were designed and synthesized as previously described (Ducani et al., Nature Methods 2013). Each pseudogene was designed for the production of single stranded DNA polynucleotides of different lengths: CRC1 (SEQ ID NO: 7) for a single polynucleotide 1316 bases long; CRC2 (SEQ ID NO: 8) for a single polynucleotide 1969 bases in length; and Oligo-mix for a pool of eight oligonucleotides of length between 81 to 91 bases in length.

All of the pseudogenes were cloned into pM1 (SEQ ID NO: 1), using XbaI and BamHI restriction sites located in between the attachment sites attB and attP of the parental plasmid. The parental plasmids containing the pseudogene were sequence verified, and used to transform E. coli bacteria (ZYCY10P3S2T). Transformed bacteria cultures were grown overnight to propagate the plasmids, then the recombination process was triggered upon induction with arabinose (to a final concentration of 0.01%), and an additional 6 hour incubation was necessary to complete the process. The minicircles (MC) were collected by standard plasmid preps and loaded on a 1.5% agarose gel containing ethidium bromide (1 μg/ml; Sigma Aldrich) for analysis control (FIG. 4). All of the recombination reactions produced mixtures of circular products, which included circular monomer minicircles (MC monomer) and multimeric minicircles (MC polymers), all of which are suitable substrates (DNA minicircles) for use in the method described herein.

### Example 3—Generation of an Oligonucleotide Pool from a Single Minicircle Template

The product of the recombination reaction involving the Oligo mix minicircle of Example 2 was enzymatically nicked using Nb.BsrDI and Nt.BspQI to provide a 3′OH to trigger the RCA reaction. The RCA reaction was performed overnight using phi29 DNA polymerase. The amplification product was later digested with BtsCI (0.5U/μl) to release the desired oligonucleotide sequences, and the digestion products were run on a 10% denaturing polyacrylamide gel, stained in SybrGold 1× (FIG. 5). The same result was achieved when the production started from a gel extracted minicircle monomer.

### Example 4—Generation of Long Single Stranded DNA Polynucleotides Using Minicircle Templates

The products of the recombination reactions involving the CRC1 and CRC2 minicircles of Example 2 were used as templates for the production of single stranded DNA polynucleotides. Both recombination products were enzymatically nicked (Nb.BsrDI and Nt.BspQI) to provide a 3′OH to trigger the RCA reactions. The RCA reactions were performed overnight using phi29 DNA polymerase. The amplification products were later digested with BtsCI (0.5U/μl) to release the desired DNA polynucleotide sequences, and the digestion products were run on a 2% agarose gel containing ethidium bromide (1 μg/ml; Sigma Aldrich) for analysis control (FIG. 6).

### Example 5—Enzymatic Production of Single Stranded Oligonucleotides Comprising Fluorescent Nucleotides

Single stranded fluorescent oligonucleotides 378 nucleotides in length (SEQ ID NO: 9) were produced enzymatically using phi29 DNA polymerase. This was done via incorporation of two different functionalised dATP nucleobases, one comprising the fluorophore Cy3 (7-Propargylamino-7-deaza-ATP-Cy3) and one comprising the fluorophore ATTO-488 (7-Propargylamino-7-deaza-ATP-ATTO-488).

A double stranded circular DNA template containing SEQ ID NO: 9 and hairpin cleavage domains was prepared as described in Ducani et al., 2013, Nature Methods, 647-652. The template (1 ng/μL) was nicked with Nb.BsrDI and Nt.BspQI (0.25 U/μL) and a rolling circle amplification reaction (0.1-0.25 ng/μL template DNA, phi29 DNA polymerase 0.25 U/μL, 0.1 pg T4 gene 32) was performed several times with different ratios of natural dATPs and functionalised dATPs in each reaction (i.e. different relative amounts of the functionalised dATP, 2%, 3% or 5%). The resulting RCA products were diluted five times in deionized water and 1× digestion buffer (50 mM Potassium Acetate, 20 mM Tris-acetate, 10 mM Magnesium Acetate, 100 μg/ml BSA, pH 7.9 at 25° C.) and were then digested with BtsCI restriction enzyme (0.5U/μL) overnight at 50° C. and the digestion products were run on agarose gels. Imaging was done using the emission wavelengths corresponding to the two fluorophores, and after ethidium bromide staining, by UV visualization.

The resulting images are shown in FIGS. 7A and B for ATTO-488 and Cy3, respectively. It can be seen that increasing the percentage of dATP-ATTO-488 nucleotides resulted in oligonucleotides with higher fluorescence. However, the total amount of RCA product dropped by approximately 60% when 5% of the dATP nucleotides were dATP-ATTO-488.

Surprisingly and in contrast to the use of dATP-ATTO-488, the percentage of dATP-Cy3 nucleotides present did not seem to affect the efficiency of phi29 DNA polymerase; single stranded DNA products were visible with 5% of dATP-Cy3 in the RCA mixture (FIG. 7B). Moreover, incorporation of the modified nucleotides did not prevent or reduce the efficiency of the BtsCI restriction enzyme, and consequently the release of the designed hairpins comprising the cleavage domains.

SEQ ID NO: 9:
CCGGCGTCAATACGGGATAATACCGCGCCACATAGCAGAACTTTA
AAAGTGCTCATCATTGGAAAACGTTCTTCGGGGCGAAAACTCTCA
AGGATCTTACCGCTGTTGAGATCCAGTTCGATGTAACCCACTCGT
GCACCCAACTGATCTTCAGCATCTTTTACTTTCACCAGCGTTTCT
GGGTGAGCAAAAACAGGAAGGCAAAATGCCGCAAAAAAGGGAATA
AGGGCGACACGGAAATGTTGAATACTCATACTCTTCCTTTTTCAA
TATTATTGAAGCATTTATCAGGGTTATTGTCTCATGAGCGGATAC
ATATTTGAATGTATTTAGAAAAATAAACAAATAGGGGTTCCGCGC
ACATTTCCCCGAAAAGTG

### Example 6—Enzymatic Production of Single Stranded Oligonucleotides Comprising Nucleotides with Internalized Alkyne Groups, i.e. Alkyne Groups in the Nucleobase

Single stranded oligonucleotides 420 nucleotides in length (SEQ ID NO: 10) comprising nucleotides with alkyne groups were produced enzymatically using phi29 DNA polymerase.

A double stranded circular DNA template containing SEQ ID NO: 10 and hairpin cleavage domains was prepared as described in Ducani et al., 2013, Nature Methods, 647-652. Nicking of the template RCA reactions were performed using the conditions described in Example 1, but using increasing relative amounts of 5′-Ethynyl-dUTP (5′-EdUTP), i.e. replacing dTTP with 5′-EdUTP such that the relative amount of 5′-EdUTP was 0% (control reaction), 25%, 50%, 75% or 100%. After amplification, the RCA products were digested by the BtsCI restriction enzyme and loaded on to an agarose gel as described in Example 5.

The results in FIG. 8A surprisingly show that even when up 100% of the dTTP nucleotides were replaced with the alkyne functionalised dUTP, the RCA yields dropped only by 15-20%. Thus, FIG. 8A also demonstrates that the RCA product was successfully and efficiently cleaved by BtsCI. The incorporation of the alkyne functionalised dUTP nucleotide was confirmed by the fact that a lower mobility of the functionalised oligonucleotide was observed.

An additional experiment was performed replacing phi29 DNA polymerase with Bst DNA polymerase. Gel electrophoresis showed no changes in amplification yield up to 50% of functionalised dUTP, with the final product shifted compared the one with 25% of modified dUTP, confirming the incorporation of the modified nucleotide which has higher molecular weight than its corresponding natural nucleotide (dTTP) (FIG. 8B).

SEQ ID NO: 10:
ATTGAAGCATGCGGCGTGCATAATTCTCTTACTGTCATGCCATGC
GTAAGATACCACCACACCCGCATTCGCCATTCAGGCGGCCGCCAC
CGCGGTGGAGCTCCAGCTGCTGTTTCCTGTGTAGAGTTGGTAGCT
CTTGATCCGGTCATATTTGTTCCCTTTAGATCCGCCTCCATCTAC
AGGGCGCGTCCCCGCGCTTAATGCGCGGCCTAACTACGGCTACAC
TAGAAGGACTTACCTTCGGAAAAGAAATTGTTATCCGCTCACAAA
AGCCAGAGTATTTAAGCTCCCTCGTGCGCTCTCCTGTTCCGGGTT
ATTGTCTCATCGGCGACCGAGTTGCTCTTGCTTATCAGACCCTGC
CGCTTACAAGTGGTCGCCAGTCTATTAACAGCACTCAATACGGGA
TAATTTTTCAATATT

### Example 7—Click Chemistry Reaction to Conjugate Azide-Fluorophore to Single Stranded Nucleotide Comprising Internalised Alkyne Groups

The successful incorporation of the functionalised 5-Ethynyl-dUTP nucleotides into the oligonucleotide produced in Example 6 was further demonstrated by performing a click chemistry reaction.

The functionalised oligonucleotide from the reaction with 75% of the alkyne functionalised dUTP nucleotide was incubated with a Cy3-azide (50 μM). The click chemistry solution also included copper sulfate (50 μM) as a catalyst, sodium ascorbate (50 mM) and THPTA (250 μM). As negative control, an oligonucleotide produced by the same method from the same template but with conventional dNTPs was also incubated with the Cy3-azide. In addition, the functionalised oligonucleotide comprising internalised alkyne groups was also incubated in the absence of the Cy3-azide. The reaction mixtures from the three reactions were run on an agarose gel and imaged (FIG. 9). Fluorescent single stranded oligonucleotides of the expected length were observed only for the reaction comprising the functionalised oligonucleotide and the fluorophore-azide. In addition, no visible DNA degradation due to the presence of the copper sulfate was observed.

### Example 8—Enzymatic Production of Single Stranded Oligonucleotides Comprising Endonuclease Resistant Nucleotides

2′-Fluoro-2′-deoxyuridine-5′-triphosphate (2′F-dUTP) or 2′-Deoxythymidine-5′-O-(1-Thiotriphosphate) (phosphorothioate dTTP) have both been previously used to modify DNA and RNA oligonucleotides for biomedicine and therapeutics applications, due to their capacity of conferring nuclease stability. These modified nucleotides were incorporated into single stranded DNA oligonucleotides by RCA using the experimental schemes described above. The conventional dTTP nucleotides were replaced by the functionalised nucleotides in increasing percentages from 0 to 100%. The tandem repeat RCA products were then digested by the BtsCI restriction enzyme into discrete 420 base single stranded functionalised oligonucleotides (SEQ ID NO: 10).

In the experiment performed with the 2′F-dUTP functionalised nucleotides, similar RCA yields were visible from 0% up to 75% of the functionalised nucleotide, with a drastic drop in yield observed with 100% of the functionalised nucleotide (FIG. 10A).

In the phosphorothioate dTTP experiment, the RCA yield decreased gradually as the amount of the functionalised nucleotide was increased, up to approximately a 65% drop with 75% of the functionalised nucleotide, relative to the yield with only conventional nucleotides (FIG. 10B).

However, overexposure of the agarose gels showed how functionalised single stranded oligonucleotides were produced even with 100% of functionalised nucleobases—see the panels on the right of FIGS. 10A and B.

Other phosphorothioate dNTPs (indicated as Alpha S dNTPs) were tested in additional experiments, either added one by one or in combination (FIG. 10C). Even in the last case, in which 75% of all the conventional nucleotides were replaced with their corresponding Alpha S functionalised nucleotides, RCA products were synthesized and enzymatically cleaved to yield oligonucleotides visible on the agarose gel.

The endonuclease resistance of the functionalised oligonucleotides relative to control oligonucleotides produced with only conventional nucleotides was investigated. A control 420-nt long oligonucleotide produced with only conventional nucleotides, the 2′-F-dUTP functionalised oligonucleotides and phosphorothioate dTTP functionalised oligonucleotides (both produced using a relative amount of 75% of the functionalised nucleotide), were incubated with increasing concentrations of DNAse I (FIGS. 11A-C). The control oligonucleotide was completely digested with 18 mU/ml of DNAse I, but both the enzymatically produced 2′-F-dUTP and phosphorothioate dTTP functionalised DNA oligonucleotides were still visible on agarose gels after incubation with the same concentration of endonuclease.

### Example 9—Enzymatic Production of Single Stranded Oligonucleotides Comprising Nucleotides with Vinyl Groups

Single stranded DNA oligonucleotides with lengths from 76-81 bases (SEQ ID NOs: 11-21), functionalised with the thymidine analogue 5-Vinyl-2′-deoxyuridine-5′-triphosphate (5-Vinyl-dUTP) were produced enzymatically via an RCA reaction according to the experimental schemes described above. All of the oligonucleotides were encoded on a single pseudogene (SEQ ID NO: 24). The incorporation of such a functionalised nucleotide in single stranded oligonucleotides enables copper-free click chemistry reactions to be used to conjugate tetrazine-like molecules to the oligonucleotide. This alkene-tetrazine reaction can be completely orthogonal to the alkyne-azide click chemistry reaction previously performed.

Increasing the amount of the functionalised nucleotide in the RCA reaction mixture, relative to the conventional nucleotide dTTP, led to the successful incorporation of the 5-Vinyl-dUTP into the single stranded RCA product and the successful digestion of the hairpin structures. However, the activity levels of both the phi29 DNA polymerase and the type II endonuclease used to cleave the RCA product were lower than in the absence of the functionalised nucleotide, which consequently led to higher molecular weight bands with undigested hairpin structures when the functionalised nucleotides fully replaced the conventional dTTP nucleotides (FIG. 12).

| SEQ ID NO | Sequence |
|---|---|
| 11 | GAACCGTCCCAAGCGTTGCGC |
| | CACATCTGCTGGAAGGTGGAC |
| | AGTGAGAGGACACCTACGAAT |
| | CGCAACGGGTATCCT |
| 12 | GAACCGTCCCAAGCGTTGCGC |
| | CTGGGTACATGGTGGTACCAC |
| | CAGACAGGACACCTACGAATC |
| | GCAACGGGTATCCT |
| 13 | GAACCGTCCCAAGCGTTGCGG |
| | AGAGCATAGCCCTCGTAGATG |
| | GGCAAGGACACCTACGAATCG |
| | CAACGGGTATCCT |
| 14 | GAACCGTCCCAAGCGTTGCGG |
| | TCCCAGTTGGTAACAATGCCA |
| | TGTTCAATGAGGACACCTACG |
| | AATCGCAACGGGTATCCT |
| 15 | GAACCGTCCCAAGCGTTGCGC |
| | GGACTCATCGTACTCCTGCTT |
| | GCTGAGGACACCTACGAATCG |
| | CAACGGGTATCCT |
| 16 | GAACCGTCCCAAGCGTTGCGT |
| | TCTCTTTGATGTCACGCACGA |
| | TTTCCCAGGACACCTACGAAT |
| | CGCAACGGGTATCCT |
| 17 | GAACCGTCCCAAGCGTTGCGC |
| | TCGGTCAGGATCTTCATGAGG |
| | TAGTCTGTAGGACACCTACGA |
| | ATCGCAACGGGTATCCT |
| 18 | GAACCGTCCCAAGCGTTGCGT |
| | TTCACGGTTGGCCTTAGGGTT |
| | CAGGGGAGGACACCTACGAAT |
| | CGCAACGGGTATCCT |
| 19 | GAACCGTCCCAAGCGTTGCGG |
| | TACTTCAGGGTCAGGATACCT |
| | CTCTTGAGGACACCTACGAAT |
| | CGCAACGGGTATCCT |
| 20 | GAACCGTCCCAAGCGTTGCGC |
| | TGCTCGAAGTCTAGAGCAACA |
| | TAGCACAAGGACACCTACGAA |
| | TCGCAACGGGTATCCT |
| 21 | GAACCGTCCCAAGCGTTGCGC |
| | CTCGTCACCCACATAGGAGTC |
| | CTTCAGGACACCTACGAATCG CAACGGGTATCCT |

### Example 10—Enzymatic Production of Single Stranded Oligonucleotides Comprising Thiolated Nucleotides

Single stranded DNA oligonucleotides functionalised with a thiolated dTTP (4-Thiothymidine-5′-Triphosphate) were produced enzymatically via an RCA reaction using the reaction scheme and the templates described above (SEQ ID NOs: 11-21). All of the oligonucleotides were encoded on a single pseudogene (SEQ ID NO: 24)

The incorporation of the thiolated nucleotide into the single stranded oligonucleotide by phi29 DNA polymerase incorporation was very successful in amounts of the functionalised nucleotide up to 75% replacement of the corresponding conventional nucleotide (dTTP) (FIG. 13), i.e. a relative amount of 75% of the functionalised nucleotide.

The RCA products were digested by type II restriction enzymes as described above, however, a complete digestion of the functionalised RCA products required an enzyme concentration 10 times higher than the concentration used on RCA products comprising only conventional nucleotides. When the conventional dTTP nucleotide was completely replaced with the functionalised thiolated nucleotide, no single stranded oligonucleotides were observed following treatment with the type II restriction enzymes, though there only very faint accumulation of undigested RCA products was observed in the well, suggesting that the activity of the polymerase was also affected.

### Example 11—Enzymatic Production of Single Stranded Oligonucleotides Comprising Azide Nucleotides

Single stranded oligonucleotides 420 nucleotides in length (SEQ ID NO: 10) comprising increasing percentages of functionalised 2′-Azido-dATP nucleotides (FIG. 15A), which replace the corresponding conventional dATP nucleotides, were produced enzymatically using phi29 DNA polymerase (FIG. 15B) and Bst DNA polymerase (FIG. 15C).

The high density azido groups in the newly synthesized DNA strands enables the post-synthesis functionalisation with alkyne molecules, either by a Cu(I)-catalyzed Huisgen cycloaddition (“click” chemistry), or by a strain-promoted [3+2] cycloaddition of azides and cycloalkynes, e.g. cyclooctyne or cyclononyne.

Two different polymerases with strand displacement activity were used in the amplification step: phi29 DNA polymerase or Bst DNA polymerase. Both polymerases were able to incorporate the functionalised nucleotide into the amplification products, which were then digested and run on an agarose gel.

DNA products produced with phi29 DNA polymerases were visible in the gel up to 75% of the modified nucleotides (FIG. 15B) while Bst DNA polymerases products were visible up to 100% (FIG. 15C), although Bst amplification buffer salts led to smear effect (even in the lane corresponding to 0% of 2′-Azido dATP). The functionalised nucleotides were incorporated successfully and did not significantly affect the formation of hairpin structures, which enable the cleavage of the amplification product.

### Example 12—Enzymatic Production of Single Stranded Oligonucleotides Comprising Biotinylated Nucleotides

Single stranded oligonucleotides 420 nucleotides in length (SEQ ID NO: 10) comprising increasing percentages (25%-100%) of functionalised Biotin-16-Aminoallyl-2′-dUTP (FIG. 16A), which replace the corresponding conventional nucleotide dTTP, were produced enzymatically using phi29 DNA polymerase (FIG. 16B).

The incorporation of the biotinylated nucleotides only slightly affected the DNA amplification reaction and, surprisingly, despite the potential for steric hindrance due to the large size of the functionalised nucleotide, did not interfere with the formation of the hairpin structures, which enable the cleavage of the RCA products. The incorporation of multiple internal biotins into a functionalised polynucleotide enables the conjugation with streptavidin functionalised molecules.

### Example 13—Enzymatic Production of Single Stranded Oligonucleotides Comprising 5-Modified Pyrimidines: 5-Bromo-2′-deoxyuridine-5′-Triphosphate and 5-Propynyl-2′-deoxycytidine-5′-Triphosphate

Single stranded oligonucleotides 420 nucleotides in length (SEQ ID NO: 10) comprising increasing percentages (25%-100%) of unnatural 5-modified pyrimidines: 5-Bromo-2′-deoxyuridine-5′-Triphosphate (FIG. 17A) and 5-Propynyl-2′-deoxycytidine-5′-Triphosphate (FIG. 17B), which replace the corresponding conventional nucleotides dTTP and dCTP, respectively, were produced enzymatically using phi29 DNA polymerase (FIGS. 17C and 17D) and Bst DNA polymerase (FIGS. 17E and 17F). Surprisingly, both functionalised nucleotides were successfully incorporated into the newly synthesized DNA sequence without affecting the formation of the hairpin structures in the RCA product, thus allowing the cleavage reaction to occur.

### Example 14—Enzymatic Production of Single Stranded Oligonucleotides Comprising 2′-O-Methyl-ATP

Single stranded oligonucleotides 420 nucleotides in length (SEQ ID NO: 10) comprising increasing percentages (25%-100%) of 2′-O-Methyl-ATP (FIG. 18A), which replace the corresponding conventional nucleotide dATP, were produced enzymatically using phi29 DNA polymerase (FIG. 18B). Even with 100% of the functionalised nucleotide present, the amplification product was still visible. This result was contrary to previous studies which showed that no known natural polymerases are capable of efficiently accepting these modified substrates (Romesberg, JACS 2004, 10.1021/ja038525p).

In addition, no higher molecular weight undigested DNA bands were visible in the gel, showing that the presence of the functionalised nucleotides did not affect the formation of the hairpin structure and its digestion by the restriction enzyme. This was a surprising result in view of the nuclease resistance which is conferred to DNA molecules by O-methyl groups.

### Example 15—Enzymatic Production of Single Stranded Oligonucleotides Comprising LNA-adenosine-5′-triphosphate

Single stranded oligonucleotides 420 nucleotides in length (SEQ ID NO: 10) comprising increasing percentages (25%-100%) of LNA-adenosine-5′-triphosphate (FIG. 19A), which replace the corresponding conventional nucleotide dATP, were produced enzymatically using phi29 DNA polymerase (FIG. 19B) and Bst DNA polymerase (FIG. 19C).

Although LNA monomers structurally mimic RNA, even with 100% of the functionalised nucleotide the efficiency of the polymerases, both phi29 DNA polymerase and Bst DNA polymerases, was not significantly affected. Moreover, the functionalised nucleotide did not interfere with the formation of the hairpin structures which enable digestion of the amplification products.

### Example 16—Generation of Single Stranded DNA from Minicircles Produced Using ParA Resolvase and FLP Recombinase

A pseudogene was designed for the production of a single stranded DNA polynucleotide, CRC1 (SEQ ID NO: 7) as described above.

The pseudogene was cloned into pM2 (SEQ ID NO: 27) and pM3 (SEQ ID NO: 28), using restriction sites located in between the recombinase attachment sites FLPr and FLPl (pM2) and msr_l and msr_r (pM3) of the respective parental plasmid. pM2 and pM3 encode FLP recombinase and ParA resolvase, respectively, under the control of an arabinose inducible promoter. The parental plasmids containing the pseudogene were sequence verified, and used to transform E. coli bacteria (DH10B). Transformed bacteria cultures were grown overnight to propagate the plasmids, then the recombination process was triggered upon induction with arabinose (to a final concentration of 0.02%), and an additional 4 hour incubation was necessary to complete the process. The minicircles (MC) were collected by standard plasmid preps and loaded on a 2% agarose gel containing ethidium bromide (1 μg/ml; Sigma Aldrich) for analysis control (FIG. 20, 1b and 2b for pM3 and pM2, respectively). The isolated minicircles were used for the enzymatic production of ssDNA as described above. FIG. 20 (1c and 2c) shows that the minicircles function as templates for RCA and subsequent cleavage of the RCA product results in ssDNA of the correct size.

1. A method for producing a plurality of single stranded DNA polynucleotides comprising:
a) providing a DNA minicircle obtained from a parental minicircle plasmid, wherein the DNA minicircle comprises the polynucleotide sequence to be produced bordered by cleavage domains;
b) performing a rolling circle amplification (RCA) reaction with the DNA minicircle of (a) as a template to produce an RCA product comprising a plurality of copies of the polynucleotide sequence to be produced, bordered by cleavage domains; and
c) cleaving the RCA product at the cleavage domains to release the plurality of single stranded DNA polynucleotides.

2. The method of claim 1, wherein step (a) comprises providing a host cell comprising the parental minicircle plasmid, wherein the host cell is capable of expressing a site-specific recombinase enzyme that acts on recombinase attachment sites in the parental minicircle plasmid.

3. The method of claim 2, wherein the site-specific recombinase enzyme is encoded by the host cell genome, optionally under the control of an inducible promoter.

4. The method of claim 2, wherein the site-specific recombinase enzyme is encoded by a plasmid in the host cell (e.g. the parental minicircle plasmid), optionally under the control of an inducible promoter.

5. The method of any one of claims 2 to 4 further comprising a step of inducing expression of the site-specific recombinase in the host cell to promote formation of the DNA minicircle in the cell.

6. The method claim 5 further comprising a step of isolating the DNA minicircle from the host cell.

7. The method of claim 1, wherein step (a) comprises contacting the parental minicircle plasmid in vitro with a site-specific recombinase enzyme that acts on recombinase attachment sites in the parental minicircle plasmid.

8. The method of any one of claims 1 to 7, wherein step (b) comprises cleaving a single strand of the DNA minicircle to provide the RCA template.

9. The method of any one of claims 1 to 8, wherein step (b) comprises hybridising a primer to the DNA minicircle.

10. The method of claim 9, wherein the primer hybridises to a sequence in the DNA minicircle that is formed by recombination of the parental minicircle plasmid.

11. The method of any one of claims 1 to 10, wherein the cleavage domains are directly adjacent to the polynucleotide sequence.

12. The method of any one of claims 1 to 11, wherein the cleavage domains contain a sequence that is recognised by a cleavage enzyme.

13. The method of any one of claims 1 to 12, wherein the cleavage domains comprise or consist of a sequence capable of forming a hairpin structure.

14. The method of claim 13, wherein the double-stranded portion of the hairpin structure comprises a sequence that is recognised by a cleavage enzyme.

15. The method of any one of claims 12 to 14, wherein the cleavage enzyme is a type II restriction endonuclease or a homing endonuclease.

16. The method of any one of claims 1 to 15, wherein the cleavage domains that border the polynucleotide sequence are cleaved by the same enzyme.

17. The method of any one of claims 1 to 16, wherein the DNA minicircle comprises a plurality of polynucleotide sequences, wherein each polynucleotide sequence is bordered by cleavage domains.

18. The method of claim 17, wherein the polynucleotide sequences are different.

19. The method of any one of claims 1 to 18, wherein the RCA reaction is performed in the presence of one or more functionalised nucleotides (dNTPs).

20. The method of any one of claims 1 to 19 further comprising a step of isolating or purifying the plurality of single stranded polynucleotides.

21. Use of a DNA minicircle obtained from a parental minicircle plasmid in the production of a plurality of single stranded DNA polynucleotides, wherein the DNA minicircle comprises the polynucleotide sequence to be produced, bordered by cleavage domains.

22. The use of claim 21, wherein the DNA minicircle is obtained by inducing the expression of a site-specific recombinase enzyme, that acts on recombinase attachment sites in the parental minicircle plasmid, in a host cell comprising the parental minicircle plasmid.

23. The use of claim 21, wherein the DNA minicircle is obtained by contacting the parental minicircle plasmid in vitro with a site-specific recombinase enzyme that acts on recombinase attachment sites in the parental minicircle plasmid.

24. The use of any one of claims 21 to 23, wherein the cleavage domains are as defined in any one of claims 11 to 16 and/or the DNA minicircle is as defined in claim 17.

25. A parental minicircle plasmid comprising:
(a) a first domain comprising:
(i) a polynucleotide sequence bordered by cleavage domains, which is bordered by recombinase attachment sites; or
(ii) an insertion site for a polynucleotide sequence bordered by cleavage domains, which is bordered by recombinase attachment sites; and
(b) a second domain comprising:
(i) two or more nickase cleavage domains, wherein each strand of the plasmid DNA comprises at least one nickase cleavage domain; and/or
(ii) a nucleotide sequence encoding a recombinase enzyme that recognises the recombinase attachment sites in the first domain.

26. The parental minicircle plasmid of claim 24 comprising:
(a) a first domain comprising:
(i) a polynucleotide sequence bordered by cleavage domains, which is bordered by recombinase attachment sites; or
(ii) an insertion site for a polynucleotide sequence bordered by cleavage domains, which is bordered by recombinase attachment sites; and
(b) a second domain comprising two or more nickase cleavage domains, wherein each strand of the plasmid DNA comprises at least one nickase cleavage domain.

27. The parental minicircle plasmid of claim 25 or 26, wherein the first domain comprises a nickase cleavage domain, optionally wherein the nickase cleavage domain in the first domain of the parental minicircle plasmid is the same as the nickase cleavage domains in the second domain of the parental minicircle plasmid.

28. The parental minicircle plasmid of claim 25 or 27, wherein the nucleotide sequence encoding a recombinase enzyme is under the control of an inducible promoter.

29. The parental minicircle plasmid of claim 28, wherein the inducible promoter is an arabinose inducible promoter.

30. The parental minicircle plasmid of any one of claims 25 to 29, wherein the parental minicircle plasmid comprises a nucleotide sequence as set forth in SEQ ID NO: 1, 27 or 28 or a nucleotide sequence with at least 80% sequence identity to a sequence as set forth in SEQ ID NO: 1, 27 or 28.

31. A kit for use in the method of any one of claims 1 to 20 comprising:
(i) a DNA minicircle obtained from a parental minicircle plasmid, wherein the DNA minicircle comprises a polynucleotide sequence bordered by cleavage domains; or
(ii) a parental minicircle plasmid as defined in any one of claims 25 to 30; and
(iii) one or more additional components for use in the method of any one of claims 1 to 20, optionally wherein the one or more additional components are: (a) one or more cleavage enzymes that cleave the cleavage domains of (i); and/or (b) functionalised dNTPs.

32. The kit of claim 31, wherein the cleavage domains are as defined in any one of claims 11 to 16 and/or the DNA minicircle is as defined in claim 17.
